(19) United States
(12) Patent Application Publication
(10) Pub. No.: US 2026/0048128 A1
(43) Pub. Date: Feb. 19, 2026

(54) SELF-THERAPEUTIC DRUG DELIVERY SYSTEM FOR NEURODEGENERATIVE DISEASES

(71) Applicant: Center for Neuromusculoskeletal Restorative Medicine Limited, Hong Kong (HK)

(72) Inventors: Chung Hang Jonathan CHOI, Hong Kong (HK); Ho Yin Edwin CHAN, Hong Kong (HK); Kit Cheung LEE, Hong Kong (HK)

(21) Appl. No.: 19/302,803

(22) Filed: Aug. 18, 2025

Related U.S. Application Data
(60) Provisional application No. 63/684,479, filed on Aug. 19, 2024.

(51) Int. Cl. A61K 47/52; A61K 47/69

(52) U.S. Cl. CPC A61K 47/52; A61K 47/6923

(57) ABSTRACT

The subject invention pertains to a novel composition comprising a self-therapeutic metal nanoparticle core coated with a hydrophilic polymer, and optionally a therapeutic agent linked to the polymer. The linker comprises functional groups that can bind to both the metal core and the polymer. The subject invention further discloses a method for treating HD in a subject comprising administering an effective amount of the composition to a subject with HD. The composition exhibits self-therapeutic properties when administered to patients with Huntington's disease. Additionally, the invention comprises a method for synthesizing gold nanoparticles for treating Huntington's disease. This method comprises conjugating gold nanoparticles with polyethylene glycol (PEG) and attaching a therapeutic agent via a linker to the PEG. This enables targeted delivery of the therapeutic agent to its therapeutic targets.

### CROSS-REFERENCE TO RELATED APPLICATION

This application claims the benefit of U.S. Provisional Application Ser. No. 63/684,479, filed Aug. 19, 2024, which is hereby incorporated by reference in its entirety including any tables, figures, or drawings.

### SEQUENCE LISTING

The Sequence Listing for this application is labeled “CNRM-100XC1-SeqList.xml” which was created on Aug. 13, 2025 and is 4,459 bytes. The entire contents of the sequence listing is incorporated herein by reference in its entirety.

### BACKGROUND OF THE INVENTION

Huntington's disease (HD) is a fatal neurodegenerative disorder characterized by progressive neurological dysfunction and neuronal loss in the striatum and cortex [1], leading to impaired motor coordination and an increased risk of suicide [2]. With a global prevalence of 2.7-5.5 per 100,000 individuals [3], HD lacks safe and effective treatments that target the underlying pathophysiological mechanisms causing neuronal cell death. Current approved drugs primarily alleviate symptoms (e.g., tetrabenazine for HD-associated chorea [4]), while some experimental drugs addressing the core disease pathology have faced clinical setbacks due to their “potential benefit/risk profile” (e.g., antisense oligonucleotides for inhibiting the mutant Huntingtin gene [5]).

Therapeutic targets, including but not limited to p38α or MAPK (p38 mitogen-activated protein kinase (MAPK)) and 3-phosphoinositide-dependent protein kinase-1 (PDK1) pathways present promising strategies for treating Huntington's disease (HD). The p38α MAPK pathway is involved in cellular stress responses, inflammation, and neuronal apoptosis, which are critical in HD pathology. Inhibiting p38α pathway could potentially reduce neuroinflammation and cell death, mitigating the progression of HD. Similarly, p38α pathway plays a crucial role in various cellular processes, including cell survival and metabolism. Targeting PDK1 may help modulate these processes and counteract the neurodegenerative effects observed in HD. By focusing on these pathways, researchers aim to address key aspects of HD pathology, such as neuroinflammation and neuronal dysfunction, to develop more effective therapeutic interventions.

Nanoparticles (NPs) are emerging as potential carriers of therapeutic agents to the brain for alleviating HD [3], with around 20-30 preclinical publications to date. Typically, NPs enhance drug delivery to the brain [6] by preventing renal clearance [7] and, when equipped with targeting ligands, by engaging receptors on the blood-brain barrier (BBB) for transcytosis [8]. However, the inclusion of carriers, drugs, and targeting ligands often results in NPs larger than 100 nm, making them susceptible to liver clearance and hindering their diffusion in the extracellular brain space [9]. NPs that are large enough to carry anti-HD drugs yet small enough to penetrate the BBB are rare.

For example, U.S. patent application Ser. No. 20/150,079007 A1 disclosed tunable nanoparticles featuring a metal nanoparticle core, such as a paramagnetic particle (e.g., USPIO) or a quantum dot, with a polymer linked to a metal-binding moiety. Although it has the capacity to be a drug carrier for suppressing cell death or pyroptosis, said patent may not have the capacity to directly modulate oxidative phosphorylation, which is critical for the brain to meet its high energy demands, support neuronal function, maintain mitochondrial health, protect against oxidative damage, and ensure proper development and plasticity. As disruptions in oxidative phosphorylation can lead to severe neurological consequences, the importance of this metabolic process in treating patients with HD is highlighted. Furthermore, said patent may have disclosed the loading of synthetic and natural drugs as one of the potential therapeutic agents and by employing additional chemicals/compounds/drugs to increase their efficacies, however, said invention may result in more severe adverse effects and increase toxicity. It also has not disclosed any capacity as self-therapeutic upon administration to the patients.

Another example of employing gold nanoparticles as a drug delivery system is disclosed in WO 2010052665 A2. The gold nanoparticles are coated with two to five layers of a combination of a polyelectrolyte with amino functionality and a polyelectrolyte with sulfonic functionality, or a single layer of the polyelectrolyte with amino functionality. While ionized polymers with amino and sulfonic functionalities offer certain advantages, they may have issues especially on stability, biocompatibility, reduced nonspecific binding, BBB penetration and reduced therapeutic efficacy.

Another example is patent JP 2017530941 A, which also disclosed the application of gold nanoparticles with PEG polymers in delivering therapeutic agents such as antioxidant, anti-inflammatory agents and/or imaging agents across the blood brain barrier and accumulating in the brain of patients having neurodegenerative diseases. The accumulation of the therapeutic agents is made possible due to the present of the triphenylphosphonium (TPP). However, it is to note that mitochondrial targeting poses risks of non-specific toxicity, compensatory mechanisms, and severe side effects due to the essential role of mitochondria in all cells. Additionally, said patent did not disclose the capacity to double as a drug carrier with self-therapeutic mechanism when administered to the patients.

Other examples of patent applications employing gold nanoparticles with PEG moieties as a delivery system was disclosed in U. S. Patent No. U.S. Pat. No. 10,182,986 B2 and Chinese Patent Application No. CN 118105357 A, with capacities of delivering one or more therapeutic agents. However, these inventions employ additional chemicals, compounds, or drugs to increase their efficacies, and due to the presence of different therapeutic targets and agents, said inventions may result in more severe adverse effects and increase toxicity.

Addressing the unresolved needs described above could lead to the development of more effective and safer therapeutic interventions for Huntington's disease by ensuring that treatments specifically target the underlying pathophysiological mechanisms without causing significant adverse effects. Consequently, such advancements would significantly improve patient outcomes, potentially slowing disease progression, preserving neurological function, and enhancing the overall quality of life for individuals living with Huntington's disease. Therefore, there is an urgent demand for safe and effective treatments targeting other HD-related pathways or therapeutic targets.

### BRIEF SUMMARY OF THE INVENTION

The present invention pertains to a novel composition comprising a metal nanoparticle for treating neurodegenerative disorders such as Huntington's disease. More specifically, the metal nanoparticle comprises a drug delivery system for a therapeutic agent and, simultaneously, the composition is also self-therapeutic when being administered to a patient having Huntington's disease.

In embodiments, the subject invention discloses a safe and effective delivery system for delivering therapeutic agents and methods of delivering therapeutic agents with capacity to modify the underlying pathophysiological mechanisms of Huntington's disease (HD), which lead to neuronal cell death.

In embodiments, the subject invention also discloses a safe and effective delivery system for delivering therapeutic agents and method of delivering therapeutic agents with the capacity to prevent renal or liver clearance and engage receptors on the blood-brain barrier (BBB) for diffusion in the extracellular brain space.

In embodiments, the composition for treating HD comprises a metal nanoparticle core coated with a hydrophilic polymer; and a therapeutic agent with a linker conjugated to the hydrophilic polymer, where the linker includes functional groups capable of binding to the metal core and the polymer. The composition also has a capacity as self-therapeutic when being administered to a patient having HD.

In embodiments, the metal nanoparticle includes, but is not limited to, Au₃@PEG₁ₖ NPs, Au₃@PEG₁ₖ NPs.

In a further aspect, the subject invention discloses a method of synthesizing a gold nanoparticle for treating Huntington's disease by conjugating gold nanoparticles with polyethylene glycol (PEG). The method further comprises conjugating a therapeutic agent with a linker to the PEG for delivering the therapeutic agent to its therapeutic target.

### BRIEF DESCRIPTION OF THE DRAWINGS

The features of the invention will be more readily understood and appreciated from the following detailed description when read in conjunction with the accompanying drawings of the preferred embodiment of the present invention, in which:

FIGS. 1A-1B illustrate TEM images of unmodified Au₃ and Au₁₃ NPs. Unmodified citrate-capped Au₃ NP of (FIG. 1A) ˜3 nm in size are synthesized by reducing HAuCl4 by sodium citrate and tannic acid; and (FIG. 1B) unmodified Au₁₃ NPs are synthesized by reducing HAuCl₄ using sodium citrate. TEM images confirm the size and shape uniformity of Auₓ NPs.

FIGS. 2A-2D illustrate TEM images of (FIG. 2A) Au₃@PEG₁ₖ NPs and (FIG. 2B) Au₃@PEG₁ₖ NPs without negative staining and (FIG. 2C) Au₃@PEG₁ₖ NP and (FIG. 2D) Au₃@PEG₁ₖ NP with negative staining to add contrast between the NP and background. Insets show the stained NPs at a higher magnification. Successful negative stain would produce contrast between the background and NPs. The PEG shell appears as a light halo around the gold core because of its lower electron scattering power relative to the gold core and the surrounding heavy-metal background stain.

FIG. 3 illustrates TGA of the lost PEG contents during the heating of Au₃@PEG₁ₖ (left) and Au₃@PEG₁ₖ (right) NPs as a function of temperature under dry nitrogen gas.

FIGS. 4A-4C illustrate UV-Vis spectra of Auₓ@PEG_{y} NPs after incubation in water and 50% fetal bovine serum (FBS) at 37° C. for 24 h wherein, (FIGS. 4A-4B) the LSPR peak of Auₓ-PEG_{y} NPs at 509-522 nm do not change drastically and no peak of NP aggregation in the 600-1000 nm range is observed upon incubation in serum and (FIG. 4C) UV-Vis spectrum of blank 50% FBS in PBS without Au NPs. Strong protein peaks at ˜400 nm resemble the ˜400 nm peaks in FIG. 4C. Note that the LSPR peaks of Auₓ@PEG_{y} NPs in the plasma of Week 10 R6/2 mice are not detectable by UV-vis spectrophotometry due to the strong absorption of the plasma.

FIGS. 5A-5B. FIG. 5A illustrates photographs of Au₃@PEG₁ₖ NP upon incubation in 50% FBS, wherein: (1) illustrates 50% FBS 24 h post-incubation at 37° C.; (2) illustrates Au₃@PEG₁ₖ NP in 50% FBS 24 h post-incubation at 37° C., and (3) illustrates Au₃@PEG₁ₖ NP in water. There is no significant color change of Au₃@PEG₁ₖ NP in FBS. FIG. 5B illustrates photographs of Au₃@PEG₁ₖ NP upon incubation in 50% FBS, wherein: (1) 50% FBS 24 h post-incubation at 37° C.; (2) Au₃@PEG₁ₖ NP in 50% FBS 24 h post-incubation at 37° C.; and (3) Au₃@PEG₁ₖ NP in water. There is no significant color change of Au₃@PEG₁ₖ NP in FBS.

FIGS. 6A-6B. (A) Photographs of Au₃@PEG₁ₖ NP upon incubation in artificial CSF. Artificial CSF 24 h post-incubation at 37° C. Au₃@PEG₁ₖ NP in artificial CSF 24 h post-incubation at 37° C. Au₃@PEG₁ₖ NP in water. There was no significant color change of Au₃@PEG₁ₖ NP in artificial, indicating colloidal stability. (B) Photographs of Au₃@PEG₁ₖ NP upon incubation in artificial CSF. Artificial CSF 24 h post-incubation at 37° C. Au₃@PEG₁ₖ NP in artificial CSF 24 h post-incubation at 37° C. Au₃@PEG₁ₖ NP in water. There was no significant color change of Au₃@PEG₁ₖ NP in artificial CSF.

FIG. 7 illustrates in-vitro cell viability of Au₃@PEG₁ₖ NP-treated SK-N-MC cells by LDH assay. The SK-N-MC cells are incubated with 200 nM Au₃@PEG₁ₖ NP for 24 h (▪), and untreated cells serve as controls (●). The amount of LDH released is normalized with that in the untreated cells. Data are from n=3, across 1 experiment. Statistical significance is evaluated using unpaired Student's t-test. All bars and error bars represent mean±SD. NP is not cytotoxic to the cells.

FIGS. 8A-8B illustrate in vitro uptake of Au₃@PEG₁ₖ NPs by 89-polyglutamine-repeats (Q89)-expressing SK-N-MC cells, model HD cells. The model HD cells are incubated with 200 nM Au₃@PEG₁ₖ NPs for 24 h, and untreated HD cells serve as controls. FIG. 8A illustrates confocal reflectance images showing the entry of Au₃@PEG₁ₖ NPs in HD model cells. FIG. 8B illustrates ICP-MS measurements verifying the association of Au₃@PEG₁ₖ NPs with HD model cells. Data are from n=3, across 1 experiment. Statistical significance is evaluated using unpaired Student's t-test. **P<0.01. All bars and error bars represent mean±SD.

FIGS. 9A-9B illustrate blood pharmacokinetics of Auₓ@PEG_{y} NP upon an i.v. injection into Week 10 R6/2 HD mice. ICP-MS measurements of Au content in blood revealed that, Au₃@PEG₁ₖ NP (FIG. 9A) shows more prolonged blood circulation than Au₃@PEG₁ₖ NP (FIG. 9B). Blood half-life time is calculated using one phase elimination model. Data are from n=2-3, across 1 experiment.

FIGS. 10A-10B illustrate photographs of Au₃@PEG₁ₖ NP upon injection in HD mice. FIG. 10A illustrates (1) plasma of uninjected Week 10 R6/2 mouse is yellow; (2) Au₃@PEG₁ₖ NP in plasma 24 h post-injection into Week 10 R6/2 mice; there is no drastic change in NP color; and (3) Au₃@PEG₁ₖ NP in water. FIG. 10B illustrates (1) plasma of uninjected Week 10 R6/2 mouse is yellow; (2) Au₃@PEG₁ₖ NP in plasma 24 h post-injection into Week 10 R6/2 mice; there is no drastic change in NP color; and (3) Au₃@PEG₁ₖ NP in water.

FIGS. 11A-11B. FIG. 11A illustrates photographs of Au₃@PEG₁ₖ NP upon injection in HD mice, wherein: (1) illustrates plasma of uninjected Week 10 R6/2 mouse is yellow; (2) illustrates Au₃@PEG₁ₖ NP in plasma 24 h post-injection into Week 10 R6/2 mice; there is no drastic change in NP color; and (3) illustrates Au₃@PEG₁ₖ NP in plasma 48 h post-injection into Week 10 R6/2 mice; the paler color suggests pronounced in vivo NP clearance from blood, a result consistent with the blood half-life of Au₃@PEG₁ₖ NPs (35 h). FIG. 11B illustrates photographs of Au₁₃@PEG₁ₖ NP upon injection in HD mice, wherein: (1) illustrates plasma of uninjected Week 10 R6/2 mouse is yellow; (2) Au₁₃@PEG₁ₖ NP in plasma 24 h post-injection into Week 10 R6/2mice; there is no drastic change in NP color; and (3) Au₁₃@PEG₁ₖ NP in plasma 48 h post-injection into Week 10 R6/2 mice; the vanished red color suggests near complete in vivo NP clearance from blood, a result consistent with the blood half-life of Au₁₃@PEG₁ₖ NPs (15 h).

FIG. 12 illustrates delivery of polyethylene glycol-conjugated gold NPs (Auₓ@PEG_{y} NPs) to the brain 24 h post intravenous (i.v.) injection into R6/2 mice as a function of NP size [Au₃@PEG₁ₖ NP (●) or Au₁₃@PEG₁ₖ NP (▪)] and age. x=NP core diameter (in nm); y=PEG molecular weight (in Da). Data are from 3-4, across 4 experiments. Statistical significance is evaluated using unpaired Student's t-test. All bars and error bars represent mean±SD.

FIGS. 13A-13C illustrate organ-level distribution for Auₓ@PEG_{y} NPs upon an i.v. injection into Week 4 (●), 6 (○), 8 (▪), and 10 (□) R6/2 HD mice. 24 h post-injection, organs of Au₃@PEG₁ₖ NP-treated R6/2 mice (FIG. 13A), Au₁₃@PEG₁ₖ NP-treated R6/2 mice (FIG. 13B) and Au₃@PEG₁ₖ NP-treated healthy littermates (FIG. 13C). The bulk gold content in each organ is detected using ICP-MS. Data are from n=3, across 6 experiments. Statistical significance is evaluated using Two-Way ANOVA with Tukey's post hoc test for multiple comparison. *P<0.05; **P<0.01; ***P<0.001; ****P<0.0001. All bars and error bars represent mean±SD.

FIGS. 14A-14D illustrate delivery of Au₃@PEG₁ₖ NP (●) and Au₁₃@PEG₁ₖ NP to the brain of R6/2 HD mice (▪) as a function of disease stage 24 h post-injection. Hippocampus (HP), cortex (CTX), striatum (CTX), cerebellum (CB) and other regions (Others). FIG. 14A shows that at the age of Week 4, there is no drastic difference between both NP sizes across different brain regions. Data are from n=3, across 1 experiment. FIGS. 14B-14D show that at the ages of Weeks 6, 8, and 10, respectively, there is more abundant uptake of Au₃@PEG₁ₖ NPs than Au₁₃@PEG₁ₖ NPs across all brain regions. Data are from n=3, across 1 experiment. All the statistical significance is evaluated using unpaired Student's t-test. As HD disease stage progresses, uptake of Au₃@PEG₁ₖ NPs in the brain of R6/2 mice increases. *P<0.05. All bars and error bars represent mean±SD.

FIG. 15 illustrates enlarged confocal reflectance images of the cryosections of CTX and STR of Week 10 R6/2 HD mice 24 h post-injection of Au₃@PEG₁ₖ NPs. Region in Red indicates brain endothelial cells (CD31), neurons (NeuN), glial cells (iba1), and astrocytes (GFAP); region in Blue indicates nucleus (DAPI); and region in Green indicates Gold NPs (green).

FIG. 16 illustrates confocal reflectance images of CTX of Week 10 healthy littermates (WT) or R6/2 HD mice 24 h post-injection of Auₓ@PEG_{y} NPs (green). Region in Red indicates brain endothelial cells (CD31), neurons (NeuN), glial cells (iba1), and astrocytes (GFAP); and region in Blue indicates nucleus. All the Auₓ@PEG_{y} NPs can enter to CTX of WT or R6/2 HD mice.

FIG. 17 illustrates confocal reflectance images of STR of Week 10 healthy littermates (WT) or R6/2 HD mice 24 h post-injection of Auₓ@PEG_{y} NPs (green). Region in Red indicates brain endothelial cells (CD31), neurons (NeuN), glial cells (iba1), and astrocytes (GFAP); and region in Blue indicates nucleus. All the Auₓ@PEG_{y} NPs can enter to STR of WT or R6/2 HD mice.

FIG. 18 illustrates distribution of Au₃@PEG₁ₖ NPs 24 h post i.v. injection into R6/2 mice or healthy littermates (o) as a function of age. Data are from n=3-4, across 4 experiments *P<0.05. Gold content in the brain is measured by ICP-MS. All bars and error bars represent mean±SD.

FIGS. 19A-19D illustrate delivery of Au₃@PEG₁ₖ NP to the brain of R6/2 HD mice (.) or control heathy littermates (WT; o) as a function of disease stage 24 h post-injection. Hippocampus (HP), cortex (CTX), striatum (CTX), cerebellum (CB) and other regions (Others). At the age of Weeks 4 (FIG. 19A), 6 (FIG. 19B), and 8 (FIG. 19C), there is no drastic difference between both mouse strains across different brain regions (except for CB at Week 8). Data are from n=3, across 1 experiment. FIG. 19D shows that at the age of Week 10, there is more abundant NP uptake in the CTX, STR, CB, O of R6/2 mice than WT mice. Data are from n=3, across 1 experiment. All the statistical significance is evaluated using unpaired Student's t-test. As HD disease stage progresses, uptake of Au₃@PEG₁ₖ NPs in the brain of R6/2 mice increases. *P<0.05. All bars and error bars represent mean±SD.

FIG. 20 illustrates permeability of the blood-brain barrier of uninjected healthy littermates (WT; ○) and Week 10 R6/2 HD mice (▴). At the age of Week 10, mice are i.v. injected with Evans blue and sacrificed 30 min post-injection. There are slightly stronger fluorescence signals in the brain lysate of R6/2 mice than WT mice. Data are from n=4, across 2 experiments. The statistical significance is evaluated using unpaired Student's t-test. *P<0.05. All bars and error bars represent mean±SD.

FIG. 21 illustrates in vivo pharmacological inhibition of caveolin and dynamin mediated brain uptake of Au₃@PEG₁ₖ NPs in Week 10 R6/2 mice. ICP-MS measurements show that sequential i.v. injection of inhibitors (filipin ( [symbol] ) or dynasore ( [symbol] )) and Au₃@PEG₁ₖ NPs to R6/2 mice reduces brain uptake of NPs 24 h post-injection. Statistical significance is evaluated using One-Way ANOVA with Dunnett's post hoc test for multiple comparison. Data are from n=3-4, across 2 experiments. * P<0.05; ** P<0.01. Gold content in the brain is measured by ICP-MS. All bars and error bars represent mean±SD.

FIG. 22 illustrates confocal reflectance images of the cryosections of CTX and STR of Week 10 R6/2 HD mice 24 h post-injection of Au₃@PEG₁ₖ NPs. Region in Red indicates brain endothelial cells (CD31); region in Blue indicates nucleus (DAPI); region in Green indicates Gold NPs (green); and region in White indicates Manders coefficient (MOC) between gold reflectance (green) and CD31 (red).

FIG. 23. Western blot analysis revealed the downregulation of the tight junction protein ZO-1 in the whole brain of Week 10 R6/2 disease mice (red) when compared to age-match, healthy wildtype (WT) littermates (blue). Data are from n=5, across one experiment. Statistical significance was evaluated using Student's t-test. **P<0.01. All bars and error bars represent mean±SD.

FIGS. 24A-24B. Role of active cellular transport in the penetration of Au₃@PEG₁ₖ NPS across a basic in vitro Transwell BBB model seeded with mouse bEnd.3 cells. After pretreating bEnd.3 cells with pharmacological blockers of various active cellular uptake pathways [filipin (olive), dynasore (cyan), chlorpromazine (brown), amiloride (blue), or sodium azide (purple)] for 1 h, the cells were further treated with medium containing both 400 μg/mL NPs and blockers for 3 more h (FIG. 24A). Pretreatment with filipin and sodium azide drastically reduced NP transport across the in vitro brain endothelium, indicating caveolae-mediated transport. “100%” indicates the total amounts of NP penetrating through the BBB without the addition of pharmacological blockers (grey). Data are from n=3, across one experiment. Cytotoxicity of the in vitro BBB endothelium upon treatment of various pharmacological blockers by the LDH assay (FIG. 24B). The amount of LDH released by NP-treated cells was normalized to that of untreated cells. Data are from n=4, across one experiment. Statistical significance was evaluated using One-Way ANOVA with Dunnett's post hoc test for multiple comparisons. *P<0.05; **P<0.01; ns=no significant difference. All bars and error bars represent mean±SD. Au₃@PEG₁ₖ NP and pharmacological blockers were not cytotoxic to bEnd.3 cells.

FIGS. 25A-25B. Role of active cellular transport in the penetration of Au₃@PEG₁ₖ NPs across a basic in vitro Transwell BBB model seeded with human hCMEC/D3 cells. After pretreating hCMEC/D3 cells with pharmacological blockers of various active cellular uptake pathways [filipin (olive) or sodium azide (purple)] for 1 h, the cells were further treated with medium containing both 400 μg/mL NPs and blockers for 3 more h. Pretreatment with filipin and sodium azide drastically reduced NP transport across the in vitro brain endothelium, indicating cavcolac-mediated transport. “100%” indicates the total amounts of NP penetrating through the BBB without the addition of pharmacological blockers (grey). Data are from n=3, across one experiment (FIG. 25A). Cytotoxicity of the in vitro BBB endothelium upon treatment of various pharmacological blockers by the LDH assay (FIG. 25B). The amount of LDH released by NP-treated cells was normalized to that of untreated cells. Data are from n=4, across one experiment. Statistical significance was evaluated using One-Way ANOVA with Dunnett's post hoc test for multiple comparisons. *P<0.05; **P<0.01; ns=no significant difference. All bars and error bars represent mean±SD. Au₃@PEG₁ₖ NP and pharmacological blockers were not cytotoxic to hCMEC/D3 cells.23A-23C

FIGS. 26A-26C. Role of active cellular transport in the penetration of Au₃@PEG₁ₖ NPS across an advanced in vitro flow-based BBB model seeded with human ESC-induced brain microvascular endothelial cells (IBMECs) in an IBAC MI microchip. Schematic illustration of IBAC MI, a gravity-driven, fluid-flow, pumpless, high-throughput microchip of the BBB (FIG. 26A). After pretreating iBMECs with pharmacological blockers of various active cellular uptake pathways [filipin (olive) or sodium azide (purple)] for 1 h, the cells were further treated with medium containing both 400 μg/mL NPs and blockers for 3 more h. Pretreatment with filipin and sodium azide drastically reduced NP transport across the in vitro brain endothelium, indicating cavcolac-mediated transport. “100%” indicates the total amounts of NP penetrating through the BBB without the addition of pharmacological blockers (grey). Data are from n=3, across one experiment (FIG. 26A). Cytotoxicity of the in vitro BBB endothelium upon treatment of various pharmacological blockers by the LDH assay (FIG. 26B). The amount of LDH released by NP-treated cells was normalized to that of untreated cells. Data are from n=4, across one experiment.

Statistical significance was evaluated using One-Way ANOVA with Dunnett's post hoc test for multiple comparisons. *P<0.05; **P<0.01; ns=no significant difference. All bars and error bars represent mean±SD. Au₃@PEG₁ₖ NP and pharmacological blockers were not cytotoxic to iBMECs.

FIG. 27. Large, stitched confocal reflectance images of the brain coronal cryosections of untreated Week 10 R6/2 HD mice and Au₃@PEG₁ₖ NP-treated Week 10 R6/2 mice 24 h post-i.v. injection. The left panel shows a large image stitched from ˜200 individual images each taken with a 40× objective. The right panels show individual fluorescence or reflection channels. Au₃@PEG₁ₖ NP was distributed throughout the brain. Red: neuron (NeuN); Blue: nucleus (DAPI); Green: gold NP.

FIG. 28. Confocal reflectance images of the cortex (CTX) and striatum (STR) of untreated R6/2 HD mice. The right column features the enlarged images of the boxed area in the left column. Red: brain endothelial cells (CD31); Blue: nucleus (DAPI); Green: gold NPs (green). No obvious non-specific signals arose from silver staining.

FIG. 29. Confocal reflectance images of the cortex (CTX) and striatum (STR) of untreated R6/2 HD mice. The right column features the enlarged images of the boxed area in the left column. Red: neurons (NeuN); Blue: nucleus (DAPI); Green: gold NPs (green). No obvious non-specific signals arose from silver staining.

FIG. 30. Confocal reflectance images of the cortex (CTX) and striatum (STR) of untreated R6/2 HD mice. The right column features the enlarged images of the boxed area in the left column. Red: activated microglia (iba1); Blue: nucleus (DAPI); Green: gold NPs (green). No non-specific signals arose from silver staining.

FIG. 31. Delivery of Au₃@PEG₁ₖ NP to choroid plexus (CP) of Week 10 R6/2 HD mice 1 h (blue) or 24 h (purple) post-i.v. injection. Density of gold content in CP was detectable as early as 1 h post-injection and became higher 24 h post-injection. CP is the site of the blood-CSF barrier, so the detectable gold signals in CP suggested the passage of gold NPs across or accumulation in the blood-CSF barrier. In terms of gold density in the brain tissue, CP is higher than other brain compartments, possibly due to the more localized deposition of gold NPs in the blood-CSF barrier and the smaller mass of CP. Data are from n=3, across two experiments. All bars and error bars represent mean±SD.

FIG. 32. Confocal reflectance images of the sagittal brain cryosections of Week 10 R6/2 mice 1 h or 24 h post-i.v. injection of Au₃@PEG₁ₖ NP. (Left) Immunostaining of epithelial cells (E-cadherin; red), coupled with the villi shape of the structure, verified the location of choroid plexus (CP) and revealed the accumulation of Au₃@PEG₁ₖ NP (green) to the CP over time, suggesting NP filtration across the blood-CSF barrier. (Right) Immunostaining of endothelial cells (CD31; red) in the brain parenchyma adjacent to CP revealed the accumulation of Au₃@PEG₁ₖ NP (green), suggesting NP penetration of the BBB. These data suggest NP brain entry of Au₃@PEG₁ₖ NP via the blood-CSF barrier. Blue: nucleus (DAPI). VS=ventricular space.

FIG. 33. Confocal reflectance images of the cryosections of the cortex (CTX) and striatum (STR) of Au₃@PEG₁ₖ NP-treated R6/2 mice sacrificed at the point of efficacy evaluation (after five weekly injections) based on the schematic shown in FIG. 2A. The right column features the enlarged images of the boxed area in the left column. Red: brain endothelial cells (CD31); Blue: nucleus (DAPI); Green: gold NPs (green).

FIG. 34. Confocal reflectance images of the cryosections of the cortex (CTX) and striatum (STR) of Au₃@PEG₁ₖ NP-treated R6/2 mice sacrificed at the point of efficacy evaluation (after five weekly injections) based on the schematic shown in FIG. 2A. The right column features the enlarged images of the boxed area in the left column. Red: activated microglia (iba1); Blue: nucleus (DAPI); Green: gold NPs (green). Confocal reflectance images of the cryosections of the cortex (CTX) and striatum (STR) of Au₃@PEG₁ₖ NP-treated R6/2 mice sacrificed at the point of efficacy evaluation (after five weekly injections) based on the schematic shown in FIG. 2A. The right column features the enlarged images of the boxed area in the left column. Red: activated microglia (iba1); Blue: nucleus (DAPI); Green: gold NPs (green).

FIG. 35. Manual counting of Au₃@PEG₁ₖ NP-containing brain cells (neurons and microglia combined) in multiple brain cryosections. By counting 500 neurons and 200 microglia in both cortex and striatum of Au₃@PEG₁ₖ NP-treated R6/2 mice sacrificed at the point of efficacy evaluation (after five weekly injections) based on the schematic shown in FIG. 2A, we found that 92% of the neurons and 78% of the microglia contained gold reflectance signals. This result indicates the presence of gold NPs in the brain parenchyma.

FIG. 36. Additional enlarged confocal reflectance images of the cryosections of the cortex (CTX) of Au₃@PEG₁ₖ NP-treated R6/2 mice sacrificed at the point of efficacy evaluation (after five weekly injections) based on the schematic shown in FIG. 2A. Red: neurons (NeuN); Blue: nucleus (DAPI); Green: gold NPs (green). These high-magnification images, similar to that in FIG. 2C but from different brain sections, depict neurons that contribute to the overall manual counting of the 500 NeuN⁺ neurons.

FIG. 37. Additional enlarged confocal reflectance images of the cryosections of the cortex (CTX) of Au₃@PEG₁ₖ NP-treated R6/2 mice sacrificed at the point of efficacy evaluation (after five weekly injections) based on the schematic shown in FIG. 2A. Red: neurons (NeuN); Blue: nucleus (DAPI); Green: gold NPs (green). These high-magnification images, similar to that in FIG. 2C but from different brain sections, depict neurons that contribute to the overall manual counting of the 500 NeuN⁺ neurons.

FIG. 38. Additional enlarged confocal reflectance images of the cryosections of the cortex (CTX) of Au₃@PEG₁ₖ NP-treated R6/2 mice sacrificed at the point of efficacy evaluation (after five weekly injections) based on the schematic shown in FIG. 2A. Red: neurons (NeuN); Blue: nucleus (DAPI); Green: gold NPs (green). These high-magnification images, similar to that in FIG. 2C but from different brain sections, depict neurons that contribute to the overall manual counting of the 500 NeuN⁺ neurons.

FIG. 39. Additional enlarged confocal reflectance images of the cryosections of the striatum (STR) of Au₃@PEG₁ₖ NP-treated R6/2 mice sacrificed at the point of efficacy evaluation (after five weekly injections) based on the schematic shown in FIG. 2A. Red: neurons (NeuN); Blue: nucleus (DAPI); Green: gold NPs (green). These high-magnification images, similar to that in FIG. 2C but from different brain sections, depict neurons that contribute to the overall manual counting of the 500 NeuN⁺ neurons.

FIG. 40. Additional enlarged confocal reflectance images of the cryosections of the striatum (STR) of Au₃@PEG₁ₖ NP-treated R6/2 mice sacrificed at the point of efficacy evaluation (after five weekly injections) based on the schematic shown in FIG. 2A. Red: neurons (NeuN); Blue: nucleus (DAPI); Green: gold NPs (green). These high-magnification images, similar to that in FIG. 2C but from different brain sections, depict neurons that contribute to the overall manual counting of the 500 NeuN⁺ neurons.

FIG. 41. Additional enlarged confocal reflectance images of the cryosections of the striatum (STR) of Au₃@PEG₁ₖ NP-treated R6/2 mice sacrificed at the point of efficacy evaluation (after five weekly injections) based on the schematic shown in FIG. 2A. Red: neurons (NeuN); Blue: nucleus (DAPI); Green: gold NPs (green). These high-magnification images, similar to that in FIG. 2C but from different brain sections, depict neurons that contribute to the overall manual counting of the 500 NeuN⁺ neurons.

FIG. 42. Confocal reflectance images of the cryosections of the 4^{th} ventricle [cerebellum (CB) and dorsal brain stem] of untreated R6/2 mice sacrificed at the point of efficacy evaluation (after five weekly injections) based on the schematic shown in FIG. 2A. The right column features the enlarged images of the boxed area in the left column. Red: Purkinje cell (Inspr3), neuron (NeuN); Blue: nucleus (DAPI). There was no obvious gold reflectance signal (green).

FIG. 43. Confocal reflectance images of the cryosections of the hippocampus (HP) and periaqueductal area of untreated R6/2 mice sacrificed at the point of efficacy based on the schematic shown in FIG. 2A. The right column features the enlarged images of the boxed area in the left column. Red: neurons (NeuN); Blue: nucleus (DAPI); Green: gold NPs (green).

FIG. 44. Confocal reflectance images of the cryosections of the cerebellum (CB) around the 4^{th} ventricle of Au₃@PEG₁ₖ NP-treated R6/2 mice sacrificed at the point of efficacy evaluation (after five weekly injections) based on the schematic shown in FIG. 2A. The right column features the enlarged images of the boxed area in the left column. Red: Purkinje cell (Inspr3), neurons (NeuN); Blue: nucleus (DAPI); Green: gold NPs (green).

FIG. 45. Confocal reflectance images of the cryosections of the dorsal brain stem around the 4^{th} ventricle of Au₃@PEG₁ₖ NP-treated R6/2 mice sacrificed at the point of efficacy evaluation (after five weekly injections) based on the schematic shown in FIG. 2A. The right column features the enlarged images of the boxed area in the left column. Red: neurons (NeuN); Blue: nucleus (DAPI); Green: gold NPs (green). FIG. 46. Confocal reflectance images of the cryosections of the hippocampus (HP) of Au₃@PEG₁ₖ NP-treated R6/2 mice sacrificed at the point of efficacy evaluation (after five weekly injections) based on the schematic shown in FIG. 2A. The right column features the enlarged images of the boxed area in the left column. Red: neurons (NeuN); Blue: nucleus (DAPI); Green: gold NPs (green).

FIG. 47. Confocal reflectance images of the cryosections of the periaqueductal area of Au₃@PEG₁ₖ NP-treated R6/2 mice sacrificed at the point of efficacy evaluation (after five weekly injections) based on the schematic shown in FIG. 2A. The right column features the enlarged images of the boxed area in the left column. Red: neurons (NeuN); Blue: nucleus (DAPI); Green: gold NPs (green).

FIG. 48. Trajectories of untreated (red), Au₃@PEG₁ₖ NP-treated (orange), tetrabenazine (TBZ)-treated (pink), 70 kDa dextran-treated (purple) R6/2 mice or untreated healthy littermates (WT) after completing an open field test for 30 min based on the treatment plan in FIG. 2A. Within the same group (column), each row shows a representative trajectory of a different animal. In each panel, the red border indicates the boundary of the open field, and the smaller green box indicates the central region of the open field.

FIG. 49. Au₃@PEG₁ₖ NP in skeletal muscles 24 h post i.v. injection in Week 10 R6/2 HD mice. Gold contents in muscles were detected using ICP-MS and expressed in terms of gold mass per tissue mass. Data are from n=3, across two experiments. Statistical significance was evaluated using One-Way ANOVA with Tukey's post hoc test for multiple comparisons. *P<0.05. All bars and error bars represent mean±SD. Tricep surac muscle (part of the hindlimb) was used for ex vivo functional tests of the muscle and neuromuscular junction due to its highest gold contents per g tissue of all muscles tested.

FIGS. 50A-50D. Ex vivo functional test of the muscle and neuromuscular junction of Week 11 R6/2 HD mice and age-matched healthy littermates. FIG. 50A illustrates a body compositional analysis by dual-energy X-ray absorptiometry (DXA) scans revealed no difference in lean mass percentage between untreated healthy littermates (WT, blue) and untreated R6/2 mice (red). FIG. 50B illustrates a schematic illustration of an ex vivo muscle functional test system. The triceps surae-sciatic nerve complex was immersed into a carboxygenated synthetic intestinal fluid (SIF) buffer bath. An electric pulse was applied by two parallel electrodes for stimulating muscle (left) or a suction electrode for stimulating nerve (right). A single pulse applied to muscle fiber results in a single contraction or “twitch”, while continuous or sustained contraction is termed “tetanic” contraction. The decline in maximal force production in response to contractile stimuli is defined as muscle fatigue, induced by repeated tetanic stimuli to assess the skeletal muscle fatigability. FIG. 50C illustrates ex vivo triceps surae muscle twitch force, tetanic force and intratetanic fatigue of untreated Week 11 R6/2 HD mice (red) and untreated age-matched healthy littermates (WT, blue) were not different, indicating limited muscle degeneration in HD mice. FIG. 50D illustrates Ex vivo triceps surae-sciatic nerve twitch force, tetanic force and intratetanic fatigue of untreated healthy littermates (WT, blue) and untreated disease mice (R6/2, red) at the age of Week 11 were not different, indicating limited degeneration of neuromuscular junction in HD mice. Data are from n=3, across one experiment. Statistical significance was evaluated using unpaired Student's t-test. ns=no significant difference (P>0.05). All bars and error bars represent mean±SD.

FIG. 51 illustrates from the age of Weeks 6 to 10, R6/2 mice receive weekly i.v. injections of Au₃@PEG₁ₖ NPs or similarly sized dextran (70 kDa), or daily intraperitoneal injections of tetrabenazine (TBZ), all sacrificed on Week 11. Behavioral tests have taken place at the age of Week 6, 7, 9 and 11.

FIG. 52 illustrates ICP-MS results showed higher brain accumulation of Au₃@PEG₁ₖ NPs in R6/2 mice (●) than Au₃@PEG₁ₖ NP-treated healthy wildtype (WT) littermates (○) on Week 11, including the cortex (CTX) and striatum (STR). Data are from n=4, across one experiment. Statistical significance is evaluated using unpaired Student's t-test. *P<0.05. All bars and error bars represent mean±SD.

FIG. 53 illustrates confocal reflectance images of the cryosections of Au₃@PEG₁ₖ NP-treated R6/2 mice sacrificed at 24 h post i.v. injection (left panel) and sacrificed at the point of efficacy based on the schematic shown in FIG. 22 (right panel). Bottom panels show enlarged 3D confocal reflectance images of the corresponding top panels. Region in Red indicates brain endothelial cells (CD31); region in Blue indicates nucleus (DAPI); and region in Green indicates Gold NPs (green).

FIG. 54 illustrates confocal reflectance images of the cryosections of the cryosections of CTX and STR of Au₃@PEG₁ₖ NP-treated R6/2 mice sacrificed at the point of efficacy based on the schematic shown in FIG. 51. The right most panel shows enlarged confocal reflectance images. Region in Red indicates brain endothelial cells (CD31), neurons (NeuN), glial cells (iba1), and astrocytes (GFAP); region in Blue indicates nucleus (DAPI); and region in Green indicates Gold NPs (green).

FIG. 55 illustrates rotarod test of untreated (▴) and Au₃@PEG₁ₖ NP-treated (●) R6/2 mice as a function of age. Data are from n=8, across three experiments. Statistical significance is evaluated using Two-Way ANOVA with Šidák post hoc test for multiple comparison. P<0.05; **P<0.01. All bars and error bars represent mean±SD.

FIG. 56 illustrates rotarod test of untreated (▴), Au₃@PEG₁ₖ NP-treated (●), TBZ-treated (*), dextran-treated ( [symbol] ) R6/2 mice at the age of Week 11. Data are from n=8-9, across four experiments. Statistical significance is evaluated using One-Way ANOVA with Tukey's post hoc test for multiple comparison. ***P<0.001, ****P<0.0001. All bars and error bars represent mean±SD.

FIG. 57 illustrates rotarod test of untreated (▴), Au₃@PEG₁ₖ NP-treated (●), tetrabenazine (TBZ)-treated (*), 70 kDa dextran-treated ( [symbol] ) R6/2 mice as a function of age, based on the treatment plan in FIG. 51. At the age of Weeks 9 and 11, Au₃@PEG₁ₖ NPs and TBZ improve the latency to fall relative to untreated control, while 70 kDa dextran does not for all ages tested. Data are from n=8-9, across four experiments. Statistical significance is evaluated using Two-Way ANOVA with Tukey's post hoc test for multiple comparison. *P<0.05; **P<0.01; ***P<0.001; ****P<0.0001. All bars and error bars represent mean±SD. There is little difference in efficacy between gold NPs and TBZ.

FIG. 58 illustrates open field test of untreated (▴), Au₃@PEG₁ₖ NP-treated (●), tetrabenazine (TBZ)-treated (*), 70 kDa dextran-treated (▾) R6/2 mice as a function of age, based on the treatment plan in FIG. 51. At the age of Week 11, both Au₃@PEG₁ₖ NPs and TBZ improve the total distance travelled in 30 min relative to untreated control, while 70 kDa dextran kDa)-treated R6/2 mice does not for all ages tested. Data are from n=6-12, across five experiments. Statistical significance is evaluated using Two-Way ANOVA with Tukey's post hoc test for multiple comparison. *P<0.05; **P<0.01. All bars and error bars represent mean±SD. There is little difference in efficacy between gold NPs and TBZ.

FIG. 59 illustrates rotarod test of untreated WT (○) and Au₃@PEG₁ₖ NP-treated WT (▪) at the age of Week 11. Data are from n=4, across one experiment. ns=no significant difference (P>0.05).

FIG. 60 illustrates rotarod test of untreated (○) and Au₃@PEG₁ₖ NP-treated (●) healthy littermates (WT) as a function of age, based on the treatment plan in FIG. 51. Data are from n=4, across 1 experiment. Statistical significance is evaluated using Two-Way ANOVA with Šidák post hoc test for multiple comparison. ns=no significant difference (P>0.05). All bars and error bars represent mean±SD. NP treatment did not change the behavior of WT mice.

FIG. 61 illustrates open field test of untreated (○) and Au₃@PEG₁ₖ NP-treated (▪) healthy littermates (WT) as a function of age, based on the treatment plan in FIG. 51. Data are from n=4, across 1 experiment. Statistical significance is evaluated using Two-Way ANOVA with Šidák post hoc test for multiple comparison ns=no significant difference (P>0.05). All bars and error bars represent mean±SD. NP treatment does not change the behavior of WT mice.

FIGS. 62A-62C illustrate Au₃@PEG₁ₖ NP-treatment reduced mHTT aggregates in R6/2 mice. FIG. 62A illustrates confocal images of MW8-stained mHTT inclusion in the CTX and STR of untreated healthy littermates (WT), untreated R6/2 HD mice and Au₃@PEG₁ₖ NP-treated R6/2 HD mice based on the treatment plan in FIG. 51, wherein White arrow indicates mHTT inclusion; Cyan indicates mHTT (MW8); and Red indicates neurons (NeuN). Quantification of the number of mHTT inclusion in NeuN-positive cells in CTX (FIG. 62B) and STR (FIG. 62C) of untreated WT (○), untreated R6/2 HD mice (▴) and Au₃@PEG₁ₖ NP-treated R6/2 HD mice (●) are illustrated. The statistical significance is evaluated using One-Way ANOVA with Tukey's post hoc test for multiple comparison. Data are from n=8, across 3 experiments. *P<0.05; **P<0.01; ***P<0.001; ****P<0.0001. All bars and error bars represent mean±SD.

FIGS. 63A-63K illustrate hematology analysis of Week 11 R6/2 HD mice that are untreated (▴) or treated by weekly injections of Au₃@PEG₁ₖ NPs from Weeks 6 to 10 (●). The markers include white blood cell count (FIG. 63A), neutrophil count (FIG. 63B), lymphocyte count (FIG. 63C), red blood cell count (RBC) (FIG. 63D), haemoglobin (FIG. 63E), haematocrit (FIG. 63F), mean corpuscular volume (MCV) (FIG. 63G), mean corpuscular hemoglobin (MCH) (FIG. 63H), mean corpuscular hemoglobin concentration (MCHC) (FIG. 63I), red cell distribution width (RDW) (FIG. 63J), and platelet count (FIG. 63K). Data are from n=4, across 1 experiment. Statistical significance is evaluated using unpaired Student's t-test. ns=no significant difference (P>0.05). All bars and error bars represent mean±SD.

FIGS. 64A-64E illustrate hepatotoxicity and renotoxicity analysis of Week 11 R6/2 HD mice that are untreated (▴) or treated by weekly injections of Au₃@PEG₁ₖ NPs from Weeks 6 to 10 (●). Serum markers include alkaline phosphatase (FIG. 64A), total bilirubin (FIG. 64B), aspartate aminotransferase (AST) (FIG. 64C), alanine transaminase (ALT) (FIG. 64D), and creatinine (FIG. 64E). Data are from n=4, across 1 experiment. Statistical significance is evaluated using unpaired Student's t-test. ns=no significant difference (P>0.05). All bars and error bars represent mean±SD.

FIGS. 65A-65D illustrate histological images of major organs in Week 11 R6/2 mice following treatment plan in FIG. 51. Au₃@PEG₁ₖ NP treatment does not alter the tissue morphology in liver (FIG. 65A), spleen (FIG. 65B), kidney (FIG. 65C), and heart (FIG. 65D). Representative images from 2 tissue sections from n=4 mice/group, across 1 experiment.

FIG. 66 illustrates schematic illustration for evaluating the long-term behavior of untreated and Au₃@PEG₁ₖ NP-treated healthy littermates (WT) and R6/2 HD (Tg) mice. From the age of Weeks 6 to 10, R6/2 mice received weekly i.v. injections of Au₃@PEG₁ₖ NPs. Behavioral tests have taken place at the age of Week 6 and starting from Week 7, the behavioral test is performed every other week. After the weekly NP injections, the mice are returned to the cage for up to 12 additional weeks and eventually sacrificed at the end stage period due to suffering of the animals.

FIG. 67 illustrates long-term survival of untreated mice [WT (solid line); R6/2 (---)] and Au₃@PEG₁ₖ NP-treated mice [WT (.-.-); R6/2 (....)] mice. Data are from n=4-9, across two experiments. P value is 0.047. Statistical significance is determined by log-rank Mantel-Cox test.

FIGS. 68A-68B illustrate end-stage rotarod tests of untreated R6/2 mice (▴) and Au₃@PEG₁ₖ NP-treated R6/2 HD mice (●) (FIG. 68A), also of untreated healthy littermates (WT, ○) and Au₃@PEG₁ₖ NP-treated WT (▪) (FIG. 68B) as a function of age, following weekly NP injections from Weeks 6 to 10 based on the treatment plan in FIG. 66. No significant difference is observed between all groups across all ages tested. Data are from n=4-5, across 1 experiment. Statistical significance is evaluated using Two-Way ANOVA with Šidák post hoc test for multiple comparison for multiple comparison. ns=no significant difference (P>0.05). All bars and error bars represent mean±SD.

FIGS. 69A-69E illustrate hepatotoxicity and renotoxicity of Week 24 R6/2 HD mice that are untreated (▴) or treated by Au₃@PEG₁ₖ NPs from Weeks 6 to 10 (●), based on serum markers. The serum markers include alkaline phosphatase (FIG. 69A), bilirubin (FIG. 69B) aspartate aminotransferase (AST, FIG. 69C), alanine transaminase (ALT, FIG. 69D), and creatinine (FIG. 69E). Data are from n=4, across 1 experiment. Statistical significance is evaluated using unpaired Student's t-test. ns=no significant difference (P>0.05). All bars and error bars represent mean±SD.

FIGS. 70A-70D illustrate histological images of major organs in Week 24 R6/2 mice following treatment plan in FIG. 66. Said major organs include liver (FIG. 70A), spleen (FIG. 70B), kidney (FIG. 70C), and heart (FIG. 70D). Au₃@PEG₁ₖ NP treatment did not alter the tissue morphology in liver, spleen, kidney and heart. Representative images from 2 tissue sections from n=3 mice/group, across 1 experiment.

FIGS. 71A-71E illustrate hepatotoxicity and renotoxicity of Week 24 healthy littermates (WT) that are untreated (○) or treated with Au₃@PEG₁ₖ NPs from Weeks 6 to 10 (▪), based on serum markers. The serum markers include alkaline phosphatase (FIG. 71A), bilirubin (FIG. 71B), aspartate aminotransferase (AST, FIG. 71C), alanine transaminase (ALT, FIG. 71D), and creatinine (FIG. 71E). Data are from n=4, across 1 experiment. Statistical significance is evaluated using unpaired Student's t-test. ns=no significant difference (P>0.05). All bars and error bars represent mean±SD.

FIGS. 72A-72D illustrate histological images of major organs in Week 24 healthy littermates following treatment plan in FIG. 66. Au₃@PEG₁ₖ NP treatment does not alter the tissue morphology in liver (FIG. 72A), spleen (FIG. 72B), kidney (FIG. 72C), and heart (FIG. 72D). Representative images from 2 tissue sections from n=4 mice/group, across 1 experiment.

FIGS. 73A-73B illustrate ICP-MS results showed that, when compared to treatment completion at the age of Week 11, the gold content in the whole brain of Au₃@PEG₁ₖ NP-treated R6/2 mice is reduced at the age of Week 24 (FIG. 73A), a trend observed in various compartments (FIG. 73B). Data are from n=3-4, across two experiments. Statistical significance is evaluated using unpaired Student's t-test. *P<0.05; **P<0.01; ***P<0.001, ****P<0.0001. All bars and error bars represent mean±SD.

FIGS. 74A-74B illustrate organ-level distribution of Au₃@PEG₁ₖ NPs in R6/2 mice and healthy littermates (WT) on Week 11 (♦) and Week 24 (×), following weekly NP injections from Weeks 6 to 10. The sum of all remaining gold contents in the bodies of R6/2 mice on Week 24 is lower than that on Week 11. FIG. 74A shows for many organs there is less gold content in R6/2 mice on Week 24 than Week 11. FIG. 74B shows data from n=3, across 2 experiments. Statistical significance is evaluated using unpaired Student's t-test. *P<0.05; ***P<0.001. All bars and error bars represent mean±SD.

FIGS. 75A-75D illustrate ICP-MS results showing that, when compared to treatment completion at the age of Week 11 (following weekly NP injections from Weeks 6 to 10), the gold contents in the whole brain (FIG. 75A) and some brain compartments (FIG. 75B) [hippocampus (HP), cerebellum (CB), and other regions (O)] of Au₃@PEG₁ₖ NP-treated healthy littermates (WT) dropped at the age of Week 24 in some but not all [cortex (CTX) and striatum (STR)]. The sum of all remaining gold contents in the bodies of WT mice on Week 24 is lower than that on Week 11. FIG. 75C shows for many organs there is less gold content in WT and on Week 24 than Week 11. FIG. 75D shows data from n=3-4, across 2 experiments. Statistical significance is evaluated using unpaired Student's t-test. *P<0.05; **P<0.01; ***P<0.001; ns=no significant difference (P>0.05). All bars and error bars represent mean±SD.

FIG. 76 illustrates volcano plot of the differentially expressed proteins (DEPs) by comparing “Au₃@PEG₁ₖ NP-treated R6/2 mice” against “untreated R6/2 mice” groups. The cut-off fold change (FC) is FC≥1.5 or FC≤1/1.5 with a P value<0.05. Bolded proteins are oxidative phosphorylation related. Data are from n=3, across one experiment.

FIG. 77 illustrates heat map of 52 differentially expressed proteins (DEPs) in the HD brain of R6/2 mice based on the comparison of Au₃@PEG₁ₖ NP group to untreated group. Treatment timeline is shown in FIG. 51. The cut-off fold change (FC) is FC≥1.5 or FC≤1/1.5 with a P value<0.05. Data are from n=3, across 1 experiment.

FIG. 78 illustrates number of enriched gene ontology (GO) terms and Kyoto Encyclopedia of Genes and Genomes (KEGG) pathways in the HD brain of R6/2 mice based on the comparison of Au₃@PEG₁ₖ NP group to untreated group. Treatment timeline is shown in FIG. 51. Enriched biological process, cellular components, molecular functions with fold changes >1.5 or <1/1.5) are shown in black, and those with P<0.05 are shown in grey. There are significant enrichments observed in 205 biological processes, 96 cellular components, 72 molecular functions, but only 8 KEGG pathways. Data are from n=3, across 1 experiment.

FIG. 79 illustrates a map of the most significant enriched Kyoto Encyclopedia of Genes and Genomes (KEGG) pathway oxidative phosphorylation identified from DEPs obtained by comparing Au₃@PEG₁ₖ NP-treated R6/2 to untreated R6/2 HD mice following the treatment schematic shown in FIG. 51. Data are from n=3, across 1 experiment.

FIG. 80 illustrates top ten enriched GO terms identified from DEPs in the HD brain of R6/2 mice based on the comparison of Au₃@PEG₁ₖ NP group to untreated group. Treatment timeline is shown in FIG. 51. The three categories of GO terms are biological process, cellular component, and molecular function. Data are from n=3, across 1 experiment.

FIG. 81 illustrates concept Network (CNET) plot of top ten GO terms in biological process. The enriched Gene Oncology terms are identified from DEPs obtained by comparing Au₃@PEG₁ₖ NP-treated R6/2 to untreated R6/2 HD mice following the treatment schematic shown in FIG. 51. On the left-hand side, the nodes represent DEPs that meet the criteria of statistical significance (P<0.05) with the cut-off fold change (FC) is FC≥1.5 or FC≤1/1.5. The color of each node corresponds to the log₂ (fold change) value. On the right-hand side, the nodes represent the top ten enriched biological processes, where the size of each node reflects the total count of DEPs associated with that specific GO term. Data are from n=3, across 1 experiment.

FIG. 82 illustrates directed acyclic graph (DAG) or ancestor chart of biological process. The enriched Gene Oncology (GO) terms are identified from DEPs obtained by comparing Au₃@PEG₁ₖ NP-treated R6/2 to untreated R6/2 HD mice following the treatment schematic shown in FIG. 51. The nodes represent a GO term with the color of the nodes corresponded to P value of the enriched GO terms with P<0.05. The solid arrow shows the relationship of “is a” between nodes. The dashed arrow shows the relationship of “part of” between nodes. Data are from n=3, across 1 experiment.

FIG. 83 illustrates concept Network (CNET) plot of top ten GO terms in cellular component. The enriched Gene Oncology terms are identified from DEPs obtained by comparing Au₃@PEG₁ₖ NP-treated R6/2 to untreated R6/2 HD mice following the treatment schematic shown in FIG. 51. On the left-hand side, the nodes represented DEPs that meet the criteria of statistical significance (P<0.05) with the cut-off fold change (FC) is FC≥1.5 or FC≤1/1.5. The color of each node corresponds to the log₂ (fold change) value. On the right-hand side, the nodes represent the top ten enriched biological processes, where the size of each node reflects the total count of DEPs associated with that specific GO term. Data are from n=3, across 1 experiment.

FIG. 84 illustrates directed acyclic graph (DAG) or ancestor chart of cellular component. The enriched Gene Oncology (GO) terms are identified from DEPs obtained by comparing Au₃@PEG₁ₖ NP-treated R6/2 to untreated R6/2 HD mice following the treatment schematic shown in FIG. 51. The nodes represent a GO term with the color of the nodes corresponds to P value of the enriched GO terms with P<0.05. The solid arrow shows the relationship of “is a” between nodes. The dashed arrow shows the relationship of “part of” between nodes. Data are from n=3, across 1 experiment.

FIG. 85 illustrates concept Network (CNET) plot of top ten GO terms in molecular function. The enriched Gene Oncology terms are identified from DEPs obtained by comparing Au₃@PEG₁ₖ NP-treated R6/2 to untreated R6/2 HD mice following the treatment schematic shown in FIG. 51. On the left-hand side, the nodes represent DEPs that meet the criteria of statistical significance (P<0.05) with the cut-off fold change (FC) is FC≥1.5 or FC≤1/1.5. The color of each node corresponds to the log₂ (fold change) value. On the right-hand side, the nodes represent the top ten enriched biological processes, where the size of each node reflects the total count of DEPs associated with that specific GO term. Data are from n=3, across 1 experiment.

FIG. 86 illustrates directed acyclic graph (DAG) or ancestor chart of molecular function. The enriched Gene Oncology (GO) terms are identified from DEPs obtained by comparing Au₃@PEG₁ₖ NP-treated R6/2 to untreated R6/2 HD mice following the treatment schematic shown in FIG. 51. The nodes represent a GO term with the color of the nodes corresponds to P value of the enriched GO terms with P<0.05. The solid arrow shows the relationship of “is a” between nodes. Data are from n=3, across 1 experiment.

FIGS. 87A-87B illustrate CTX (FIG. 87A) and STR (FIG. 87B), ATP production is enhanced for Au₃@PEG₁ₖ NP-treated (▴) than untreated (●) R6/2 mice. Data are from n=6, across two experiments. Statistical significance is evaluated using one-way ANOVA with Tukey's post hoc test for multiple comparison. *P<0.05; **P<0.01; ****P<0.0001. All bars and error bars represent mean±SD.

FIGS. 88A-88B illustrate CTX (FIG. 88A) and STR (FIG. 88B), the NAD⁺ to NADH ratio is higher for Au₃@PEG₁ₖ NP-treated than untreated R6/2 mice. Data are from n=6, across two experiments. Statistical significance is evaluated using one-way ANOVA with Tukey's post hoc test for multiple comparison. *P<0.05; **P<0.01; ***P<0.001, ****P<0.0001. All bars and error bars represent mean±SD.

FIGS. 89A-89B illustrate CTX (FIG. 89A) and STR (FIG. 89B), the levels of HNE and MDA (markers of lipid peroxidation) are lower for Au₃@PEG₁ₖ NP-treated (●) than untreated (▴) R6/2 mice. Data are from n=6-7, across one experiment. Statistical significance is evaluated using one-way ANOVA with Tukey's post hoc test for multiple comparison. *P<0.05; **P<0.01; ***P<0.001, ****P<0.0001. All bars and error bars represent mean±SD.

FIGS. 90A-90C illustrates 10-point titration curves and IC₅₀ values of Au₃@PEG₁ₖ NPs or 70 kDa dextran NP on MAPK14 (p38α) (FIG. 90A), MAP2K6 (MKK6; upstream of p38α) (FIG. 90B), and PDK1 (FIG. 90C) respectively. 70 kDa dextran is a “size-matched” negative control of Au₃@PEG₁ₖ NP; both are ˜11 nm in diameter.

FIGS. 91A-91E FIG. 91A illustrates the Western blot analysis of health mice (WT), disease mice (R6/2) and Au₃@PEG₁ₖ NP-treated R6/2 mice. FIGS. 91B-91E further reveal the (i) activation of p-PDHα1 (a downstream kinase of PDK1) and p-p38α in R6/2 mice (▴) when compared to healthy WT littermates (○) and (ii) inhibition of both kinases upon Au₃@PEG₁ₖ NP treatment of R6/2 mice (●) when compared to untreated R6/2 mice (▴) in CTX (FIGS. 6912B-91C) and STR (FIGS. 91D-91E). Data are from n=4, across one experiment. (FIGS. 91B-91E) Quantification of the western blot data in (FIG. 91A) for the expression of p-PDHα1 (relative to PDHα1) and p-p38α (relative to total p38α). Statistical significance is evaluated using one-way ANOVA with Tukey's post hoc test for multiple comparison. *P<0.05; **P<0.01; ***P<0.001, ****P<0.0001. All bars and error bars represent mean±SD.

FIG. 92 illustrates rotarod test of untreated (▴), Au₃@PEG₁ₖ NP-treated (●), and p38α MAPK-IN-1 (an inhibitor of p38α)-treated (x) R6/2 mice as a function of age, based on the treatment plan in FIG. 51. At Week 7, Au₃@PEG₁ₖ NP-treated R6/2 mice show improved motor activity. On Weeks 9 and 11, both Au₃@PEG₁ₖ NP-and p38α MAPK-IN-1 treatments improve motor activity than untreated. Data are from n=4-8, across four experiments. Statistical significance is evaluated using Two-Way ANOVA with Tukey's post hoc test for multiple comparison. *P<0.05; **P<0.01; ***P<0.001. All bars and error bars represent mean±SD.

FIG. 93 illustrates rotarod test of untreated (▴), Au₃@PEG₁ₖ NP-treated (●), and dichloroacetate (DCA; an inhibitor of PDK1)-treated (+) R6/2 mice as a function of age, based on the treatment plan in FIG. 51. At Week 7, Au₃@PEG₁ₖ NP-treated R6/2 mice showed improved motor activity. On Weeks 9 and 11, both Au₃@PEG₁ₖ NP-and DCA treatments improve motor activity than untreated. Data are from n=4-8, across four experiments. Statistical significance is evaluated using Two-Way ANOVA with Tukey's post hoc test for multiple comparison. *P<0.05; **P<0.01; ***P<0.001. All bars and error bars represent mean±SD.

FIG. 94 illustrates open field test of untreated (▴), Au₃@PEG₁ₖ NP-treated (●), and p38α MAPK-IN-1 (an inhibitor of p38α)-treated (x) R6/2 mice as a function of age, based on the treatment plan in FIG. 51. At Week 11, both Au₃@PEG₁ₖ NP-and p38α MAPK-IN-1 treatments improve the total distance travelled in 30 min than untreated. Data are from n=4-12, across five experiments. Statistical significance is evaluated using Two-Way ANOVA with Tukey's post hoc test for multiple comparison. *P<0.05, **P<0.01. All bars and error bars represent mean±SD.

FIG. 95 illustrates open field test of untreated (▴), Au₃@PEG₁ₖ NP-treated (●), and dichloroacetate (DCA; an inhibitor of PDK1)-treated (+) R6/2 mice as a function of age, based on the treatment plan in FIG. 51. At Week 11, both Au₃@PEG₁ₖ NP-and DCA treatments improved the total distance travelled in 30 min than untreated. Data are from n=4-12, across five experiments. Statistical significance is evaluated using Two-Way ANOVA with Tukey's post hoc test for multiple comparison. **P<0.01. All bars and error bars represent mean±SD.

FIGS. 96A-96C FIG. 96A illustrates Western blot and FIGS. 96B-96C illustrate quantification of FIG. 96A and reveal the (i) activation of p-PDHα1 (a downstream kinase of PDK1) in R6/2 mice (▴) when compared to healthy WT littermates (○) and (ii) inhibition of p-PDHα1 upon dichloroacetate (DCA; pharmacological inhibitor of PDK) treatment of R6/2 mice (+) when compared to untreated R6/2 mice (A) in CTX (FIG. 96B) and STR (FIG. 96C). Data are from n=4, across one experiment. Statistical significance is evaluated using one-way ANOVA with Tukey's post hoc test for multiple comparison. *P<0.05; **P<0.01. All bars and error bars represent mean±SD.

FIGS. 97A-97C FIG. 97A illustrates Western blot analysis and FIGS. 97B-97C illustrate quantification of FIG. 97A and revealed the (i) activation of p-p38α in R6/2 mice (▴) when compared to healthy WT littermates (○) and (ii) inhibition of p-p38α upon p38α MAPK-IN-1 (pharmacological inhibitor of p38α) treatment of R6/2 mice (x) when compared to untreated R6/2 mice (A) in CTX (FIG. 97B) and STR (FIG. 97C). Data are from n=4, across one experiment. Statistical significance is evaluated using one-way ANOVA with Tukey's post hoc test for multiple comparison. *P<0.05; **P<0.01. All bars and error bars represent mean±SD.

FIGS. 98A-98B illustrate Caspase 3 like activity of the tissue homogenate of healthy littermates (○), untreated R6/2 mice (▴), Au₃@PEG₁ₖ NP-treated (●) R6/2 mice. The treatment schematic of the study followed FIG. 51. There is no reduction of Caspase 3 like activity in Au₃@PEG₁ₖ NP-treated (●) R6/2 mice in both CTX (FIG. 98A) and STR (FIG. 98B). Data are from n=5-7, across 1 experiment. The statistical significance is evaluated using One-Way ANOVA with Tukey's post hoc test for multiple comparison. *P<0.05; **P<0.01; ***P<0.001; ****P<0.0001; ns=no significant difference. All bars and error bars represent mean±SD.

FIGS. 99A-99E FIG. 99A illustrates Western blot analysis and FIGS. 99B-99E illustrate inhibition of NLRP-3 and cleaved GSDMD (markers of pyroptosis) upon treatment of R6/2 mice (●) with Au₃@PEG₁ₖ NPs when compared to untreated R6/2 mice (▴) in CTX (FIGS. 99B-99C) and STR (FIGS. 99D-99E); region in o indicates the levels of uninjected healthy littermates. Data are from n=4, across one experiment. FIGS. 99B-99E further illustrate quantification of the western blot data in FIG. 99A for the expression of NLRP-3 and cleaved GSDMD to ß-tubulin.

FIGS. 100A-100D illustrate Au₃@PEG₁ₖ NP reduced neuroinflammation in the CTX of R6/2 mice. FIG. 100A illustrates Western blot analysis and FIG. 100B illustrates the quantification which revealed reduction of iba1 in the CTX due to Au₃@PEG₁ₖ NP treatment. Data are from n=4, across one experiment. FIG. 100C illustrates confocal images of iba1-stained (bottom-left; ▴) CTX cryosections of Au₃@PEG₁ₖ NP-treated R6/2 mice arca. FIG. 100D illustrates quantification of the iba1-positive area in FIG. 100B. Data are from n=4-5, across 2 experiments. Blue=nucleus. Statistical significance is evaluated using one-way ANOVA with Tukey's post hoc test for multiple comparison. *P<0.05; **P<0.01; ***P<0.001. All bars and error bars represent mean±SD.

FIGS. 101A-101D illustrate Au₃@PEG₁ₖ NP reduced neuroinflammation in the CTX of R6/2 mice. FIG. 101A illustrates Western blot analysis and FIG. 101B illustrates the quantification revealed reduction of iba1 in the CTX due to Au₃@PEG₁ₖ NP treatment. Data are from n=4, across one experiment. FIG. 101C illustrates confocal images of iba1-stained (bottom-left; red) CTX cryosections of Au₃@PEG₁ₖ NP-treated R6/2 mice arca. FIG. 101D illustrates quantification of the iba1-positive arca in FIG. 101B. Data are from n=4-5, across 2 experiments. Blue=nucleus. Statistical significance is evaluated using one-way ANOVA with Tukey's post hoc test for multiple comparison. *P<0.05; **P<0.01. All bars and error bars represent mean±SD.

FIGS. 102A-102H illustrate ELISA analysis reveals inhibition of pyroptosis-related cytokines upon Au₃@PEG₁ₖ NP treatment of R6/2 mice (●) when compared to untreated R6/2 mice (▴) in CTX and STR, such as IL-1β (FIGS. 102A-102B), TNF-α (FIGS. 102C-102D), IL-4 (FIGS. 102E-102F), and IL-6 (FIGS. 102G-102H). Data are from n=4-8, across one experiment. All statistical significance is evaluated using one-way ANOVA with Tukey's post hoc test for multiple comparison. *P<0.05; **P<0.01; ***P<0.001, ****P<0.0001. All bars and error bars represent mean±SD.

FIG. 103. Liver accumulation of Au₃@PEG₁ₖ NP upon i.v. injection into Week 4 (blue), 6 (purple), 8 (pink) and 10 (green) R6/2 mice 24 h post-injection. The bulk gold content in each organ was detected using ICP-MS and expressed in terms of gold mass per tissue mass. Data are from n=3, across 6 experiments. Statistical significance was evaluated using One-Way ANOVA with Tukey's post hoc test for multiple comparisons. ns=no significant difference. All bars and error bars represent mean±SD. These absolute values are high when compared to previous work, possibly because of our high gold NP dose to mitigate liver clearance in terms of % ID and achieve therapeutic efficacy; this NP dose is similar to that of chiral gold NP for treating Alzheimer's disease.

FIG. 104. Proposed therapeutic mechanism of Au₃@PEG₁ₖ NPs in HD mice. Schematic illustration of proposed therapeutic mechanisms of Au₃@PEG₁ₖ NP to HD followed by weekly i.v. injections of Au₃@PEG₁ₖ NPs into R6/2 mice. Red arrows indicate the up/down-regulated protein/pathway state or action of Au₃@PEG₁ₖ NPs; black arrows indicate the up/down-regulated protein/pathway state or relationship between proteins/pathways suggested in the literature. In HD, it has been demonstrated in the literature that the transcription of mHITT gene gives rise to the phosphorylation of PDK1 which phosphorylates PDH to its inactive form. As a result, the cells of HD will undergo anaerobic respiration instead of proceeding to oxidative phosphorylation and this will lead to reduced ATP production. Furthermore, mitochondrial dysfunction will further lead to oxidative stress and NLRP-3 inflammasome-mediated neuroinflammation via activation of caspase 1. The release of IL-1β will further elicit inflammation and the cleavage of GSDMD by activated caspase I will further form pores on cell death, causing pyroptosis-mediated cell death.

In addition, the upregulation of MAPK signalling pathways [i.c., phosphorylation of mitogen-activated protein kinase kinase 6 (MKK6), p38α and Mitogen-activated protein kinase-activated protein kinase 2 (MK2)] will further trigger neuroinflammation. To revert pyroptosis-mediated cell death, Au₃@PEG₁ₖ NP can upregulate oxidative phosphorylation and protect mitochondria from triggering pyroptosis by inhibition of PDK1. Au₃@PEG₁ₖ NP can also inhibit MAPK signalling pathways and the associated neuroinflammation. The figure was created using BioRender.

FIG. 105 illustrates schematic for synthesis of Au₃@PEG₁ₖ@FA NP.

FIG. 106 illustrates TEM images of the NPs

FIG. 107 illustrates UV-vis spectra of the NPs.

FIG. 108 illustrates delivery of polyethylene glycol-conjugated Au NPs [(Au₃@PEG₁ₖ NPs (●) and Au₃@PEG₁ₖ@FA NP (▪)] to the brain 24 h post intravenous (i.v.) injection into R6/2mice as a function of FA molecule and age. Data are from n=3, across 1 experiment. All the statistical significance is evaluated using unpaired Student's t-test. All bars and error bars represent mean±SD.

FIGS. 109A-109D illustrate delivery of Au₃@PEG₁ₖ NP (●) and Au₃@PEG₁ₖ@FA NP (▪) to the brain of R6/2 HD mice as a function of FA molecule and disease stage 24 h post-injection. Hippocampus (HP), cortex (CTX), striatum (CTX), cerebellum (CB), and other regions (Others). At the age of Week 4 (FIG. 109A), 6 (FIG. 109B), 8 (FIG. 109C), and 10 (FIG. 109D), there is no drastic difference between both NP across different brain regions (except CTX at Week 4 and STR at Week 8). Data are from n=3, across 4 experiments. All the statistical significance is evaluated using unpaired Student's t-test. *P<0.05. All bars and error bars represent mean±SD.

FIG. 110 illustrates organ-level distribution for Au₃@PEG₁ₖ NP (●) and Au₃@PEG₁ₖ@FA NP (▪) upon an i.v. injection into Week 10 R6/2 mice. The bulk gold content in each organ is detected using ICP-MS. Data are from n=3, across 2 experiments. All the statistical significance is evaluated using unpaired Student's t-test. *P<0.05. All bars and error bars represent mean±SD.

FIG. 111 illustrates blood pharmacokinetics of Au₃@PEG₁ₖ@FA NP upon an i.v. injection into Week 10 R6/2 HD mice. ICP-MS measurements of Au content in blood reveal half-life of 5.8 h. Data are from n=3, across 1 experiment.

FIG. 112 illustrates rotarod test of untreated (▴), Au₃@PEG₁ₖ@FA NP-treated (▪), free FA-treated ( [symbol] ) and Au₃@PEG₁ₖ NP-treated (●) R6/2 mice as a function of age, based on the treatment plan in (FIG. 51). At the age of Weeks 9 and 11, Au₃@PEG₁ₖ@FA NP improves the latency to fall relative to untreated control, while free FA does not for all ages tested. In addition, at Week 11, Au₃@PEG₁ₖ@FA NP show improved latency to fall relative to free FA-treated group. Data are from n=7-8, across 5 experiments. Statistical significance is evaluated using Two-Way ANOVA with Tukey's post hoc test for multiple comparison. *P<0.05; **P<0.01. All bars and error bars represent mean±SD.

FIG. 113 illustrates delivery of 700 μg Au₃@PEG₁ₖ NP (●) and 175 μg Au₃@PEG₁ₖ@FA NP (★) to the brain of R6/2 HD mice as a function of dose and FA molecule. Data are from n=3, across 2 experiments. ICP-MS measurement can only barely detect trace amount of Au in 175 μg Au₃@PEG₁ₖ @FA NP-treated group 24 h post injection. Statistical significance is evaluated using unpaired Student's t-test. ****P<0.0001. All bars and error bars represent mean±SD.

FIG. 114 illustrates delivery of 700 μg Au₃@PEG₁ₖ NP (●) and 175 μg Au₃@PEG₁ₖ@FA NP (★) to the liver of R6/2 HD mice as a function of dose and FA molecule. Both injections show similar liver uptake 24 h post injection. Data are from n=3, across 2 experiments. Statistical significance is evaluated using unpaired Student's t-test. ns=no significant difference (P>0.05). All bars and error bars represent mean±SD.

FIG. 115 illustrates rotarod test of untreated (●), 700 μg Au₃@PEG₁ₖ@FA NP-treated (▪) and 175 μg Au₃@PEG₁ₖ@FA NP-treated (★) R6/2 mice as a function of age, based on the treatment plan in (FIG. 51). At the age of Weeks 11, 700 μg Au₃@PEG₁ₖ@FA NP improves the latency to fall relative to untreated control and 175 μg Au₃@PEG₁ₖ @FA NP-treated group, which does not for all ages tested. Data are from n=5-8, across 4 experiments. Statistical significance is evaluated using Two-Way ANOVA with Tukey's post hoc test for multiple comparison. *P<0.05; **P<0.01. All bars and error bars represent mean±SD.

FIGS. 116A-116B illustrate following the treatment schematic in (FIG. 51), 8-OHdG ELISA assay revealed no significant increase in 8-OHdG level in the cortex (CTX) of R6/2 mice as compared to healthy littermates (WT) (FIG. 116A). Data are from n=4, across 1 experiment. Statistical significance is evaluated using unpaired Student's t-test. As illustrated in FIG. 116B, 8-OHdG ELISA assay shows significant increase in 8-OHdG level in the striatum (STR) R6/2mice as compared to WT while Au₃@PEG₁ₖ@FA NP treatment can significantly reduce the 8-OHdG level in the STR of R/6 mice. There is no significant decrease in 8-OHdG level in the STR of both FA-treated and Au₃@PEG₁ₖ NP-treated R6/2 mice. Data are from n=4, across 1 experiment. Statistical significance is evaluated using one-way ANOVA with Tukey's post hoc test for multiple comparison. *P<0.05; ns=no significant difference. All bars and error bars represent mean±SD.

FIGS. 117A-117D illustrate immunohistochemistry of 8-OHdG in paraffin sections of healthy littermates (WT; ○), untreated R6/2 mice (▴) and Au₃@PEG₁ₖ @FA NP-treated R6/2 mice (▪) following the treatment schematic in FIG. 51. FIG. 117A illustrates representative immunohistochemistry images of 8-OHdG in the striatal regions of all the untreated and treatment groups. FIG. 117B illustrates quantification of FIG. 117A by counting the number of 8-OHdG⁺ cells per image. FIG. 117C illustrates quantification of FIG. 117A by measuring the optical density of 8-OHdG in each of the striatal cells. FIG. 117D illustrates frequency distribution of FIG. 117C. Data are from n=7-10, across 2 experiments. All the statistical significance is evaluated using one-way ANOVA with Tukey's post hoc test for multiple comparison. **P<0.01; ***P<0.001, ****P<0.0001. All bars and error bars represent mean±SD.

FIGS. 118A-118B illustrate lipid peroxidation assay showed increased HNE and MDA level in both cortex (FIG. 118A, CTX) and striatum (FIG. 118B, STR) of R6/2 mice when compared to health littermates (WT, ○). Following the treatment schematic in FIG. 51, lipid peroxidation showed reduced lipid peroxidation in both CTX and STR of Au₃@PEG₁ₖ@FA NP-treated (▪) and Au₃@PEG₁ₖ NP-treated (●) group compared to untreated R6/2 mice (▴). In free FA-treated group ( [symbol] ), a decrease in lipid peroxidation can only observed in CTX. In STR, Au₃@PEG₁ₖ@FA NP outperformed Au₃@PEG₁ₖ NP treatment. Data are from n=7-10, across 2 experiments. All the statistical significance is evaluated using one-way ANOVA with Tukey's post hoc test for multiple comparison. *P<0.05; **P<0.01; ***P<0.001, ****P<0.0001. All bars and error bars represent mean±SD.

FIGS. 119A-119H illustrate ELISA analysis of tissue lysate of healthy littermates (○), untreated R6/2 mice (▴), Au₃@PEG₁ₖ NP-treated (●) and Au₃@PEG₁ₖ@FA NP-treated (▪) R6/2 mice. It revealed inhibition of most of the cytokine upon Au₃@PEG₁ₖ@FA NP treatment of R6/2 mice when compared to untreated R6/2 mice in CTX and STR, such as TNF-α (FIGS. 119A-119B), IL-1β (FIGS. 119C-119D), IL-4 (FIGS. 119E-119F), and IL-6 (FIGS. 119G-119H). In CTX, Au₃@PEG₁ₖ@FA NP treatment of R6/2 mice outperforms Au₃@PEG₁ₖ NP treatment. In STR, Au₃@PEG₁ₖ@FA NP treatment of R6/2 mice shows significant reduction in all the cytokines except IL-1β and IL-6. Data are from n=4-8. All the statistical significance is evaluated using one-way ANOVA with Tukey's post hoc test for multiple comparison. *P<0.05; **P<0.01; ***P<0.001, ****P<0.0001. All bars and error bars represent mean±SD.

FIGS. 120A-120C illustrate hepatotoxicity and renotoxicity analysis of Week 11 R6/2 HD mice that are untreated (▴) or treated by weekly injections of Au₃@PEG₁ₖ@FA NPs from Weeks 6 to 10 (▪). Serum markers include alkaline phosphatase (FIG. 120A), aspartate aminotransferase (AST) (FIG. 120B), and creatinine (FIG. 120C). Data are from n=3-4, across 1 experiment. Statistical significance is evaluated using unpaired Student's t-test. ns=no significant difference (P>0.05). All bars and error bars represent mean±SD.

FIGS. 121A-121K illustrate hematology analysis of Week 11 R6/2 HD mice that are untreated (▴) or treated by weekly injections of Au₃@PEG₁ₖ@FA NPs from Weeks 6 to 10 (▪). The markers include white blood cell count (FIG. 121A), neutrophil count (FIG. 121B), lymphocyte count (FIG. 121C), red blood cell count (RBC, FIG. 121D), haemoglobin (Figure 121E), haematocrit (FIG. 121F), mean corpuscular volume (MCV, FIG. 121G), mean corpuscular haemoglobin (MCH, FIG. 121H), mean corpuscular hemoglobin concentration (MCHC, FIG. 121I), red cell distribution width (RDW, FIG. 121J), and platelet count (FIG. 121K). Data are from n=4, across 1 experiment. Statistical significance is evaluated using unpaired Student's t-test. ns=no significant difference (P>0.05). All bars and error bars represent mean±SD.

### BRIEF DESCRIPTION OF THE SEQUENCES

SEQ ID NO: 1
5′-CCGCTCAGGTTCTGCTTTTA-3′ (forward primer)
SEQ ID NO: 2
5′-TGGAAGGACTTGAGGGACTC-3′ (reverse primer)
SEQ ID NO: 3
5′-CTA GGCCACAGA ATTGAA AGATCT-3′ (forward
primer, internal positive control)
SEQ ID NO: 4
5′-GTAGGTGGAAATTCTAGCATCATCC-3′ (reverse
primer, internal positive control)

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” are used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

As used herein, a “pharmaceutical” refers to a compound manufactured for use as a medicinal and/or therapeutic drug.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, or improving a condition, disease, or disorder in a subject. In other words, when administered to a subject, the amount is “therapeutically effective.” The actual amount will vary depending on a number of factors including, but not limited to, the particular condition, disease, or disorder being treated, prevented, or improved; the severity of the condition; the weight, height, age, and health of the patient; and the route of administration.

The term “pharmaceutically acceptable” as used herein means compatible with the other ingredients of a pharmaceutical composition and not deleterious to the recipient thereof.

As used herein, the term “treatment” refers to eradicating; reducing; inhibiting, ameliorating; abatement; remission; diminishing of symptoms or delaying the onset of symptoms; slowing in the rate of degeneration or decline; making the final point of degeneration less debilitating; and/or improving a subject's physical or mental well-being or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, inhibiting, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

As used herein, “subject” refers to an animal, such as a mammal, for example a human. The methods described herein can be useful in both humans and non-human animals. In some embodiments, the subject is a mammal (such as an animal model of disease), and in some embodiments, the subject is a human. The terms “subject” and “patient” can be used interchangeably. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, the terms “determining,” “measuring,” and “assessing,” and “assaying” are used interchangeably and include both quantitative and qualitative determinations.

As used herein, the term “droplet”, “nanoparticle”, are intended to include their plural form as well.

For the purposes of promoting and understanding the principles of the invention, reference will now be made to the embodiments illustrated in the drawings and described in the following written specification. It is understood that the present invention includes any alterations and modifications to the illustrated embodiments and includes further applications of the principles of the invention as would normally occur to one skilled in the art to which the invention pertains.

### Composition and Methods

The present invention pertains to a novel self-therapeutic composition for treating Huntington's disease (HD) in a subject comprising a metal nanoparticle core coated with a hydrophilic polymer and a therapeutic agent with a linker conjugated to the hydrophilic polymer, wherein the linker includes functional groups capable of binding to the metal core and the polymer.

In one aspect, the subject invention discloses a composition comprising a drug carrier adapted for crossing the blood-brain barrier (BBB) of a subject with HD for delivery in the extracellular brain space and for preventing renal or liver clearance. In certain embodiments, the metal NP comprises a gold core. In embodiments, the gold core has a size/diameter of about from 2.7 to about 3.3 nm. In preferred embodiments, the gold core has a size/diameter of about 3.0 nm. In certain embodiments, the gold core has a size of about from 2.7 to about 12.7 nm. In further embodiments, the gold core has a size of about from 3.3 to about 12.7 nm. In more preferred embodiments, the gold core has a size of about 3-nm or about 13-nm.

In certain embodiments, the hydrophilic polymer coating comprises thiolated polyethylene glycol (PEG)-strands. In embodiments, the drug carrier comprises polyethylene glycol (PEG)-coated gold NP having a size/diameter ranging from about 10 nm to about 30 nm. In preferred embodiments, the (PEG)-coated gold NP has a size/diameter ranging from about 10 nm to about 25 nm. In more preferred embodiments, the (PEG)-coated gold NP has a size/diameter ranging from about 10 nm to about 20 nm. In most preferred embodiments, the (PEG)-coated gold NP has a size/diameter of about 11 nm.

In certain embodiments, the metal NP is Auₓ@PEG_{y} NP, where x ranges from 3 to 13 and y ranges from about 100-Da to 10K-Da.

In certain embodiments, the metal NP includes, but is not limited to, Au₃@PEG₁ₖ NP and Au₁₃@PEG₁ₖ NP. In preferred embodiments, the metal NP strands are conjugated with Folic Acid (FA) to obtain Au₃@PEG₁ₖ@FA NPs. The Au₃@PEG₁ₖ@FA NP has a size/diameter of about 2.7±0.5 nm.

In a preferred embodiment, the self-therapeutic composition of the subject invention comprises a drug carrier adapted for crossing the blood-brain barrier (BBB) for delivery in the extracellular brain space and for preventing renal or liver clearance in a subject, wherein the drug carrier includes, but is not limited to, a metal nanoparticle (NP), wherein the nanoparticle includes a metal core and a coating including a hydrophilic polymer and a therapeutic agent comprising a linker conjugated to the hydrophilic polymer, wherein the linker includes, but is not limited to, one or more functional groups for binding to the metal core and to the polymer.

In another aspect, the subject invention discloses a method for treating HD comprising administering an effective amount of the composition to a subject with HD. In preferred embodiments, the composition is administered intravenously (i.v.) to the subject for delivery to the brain for treating HD. In embodiments, the polyethylene glycol (PEG)-coated gold nanoparticle (NP) comprises thiolated polyethylene glycol (PEG) strands. The about 2.7-3-nm gold core enables the dissection of bio-nano interactions in the HD brain and keeps the overall NP small for BBB crossing while the shell of 1000-Da PEG strands ensures stability in blood of the gold core and promotes diffusion within the brain. In preferred embodiments, upon i.v. injection into the subject the NP enters different cell types (neuron, glial cell, astrocyte, and endothelial cell) in the cortex and striatum. Brain delivery depends on NP size and disease stage. Surprisingly, repeated injections of the NP improve motor function as effectively as tetrabenazine and prolong survival without the aid of known chemical or biological anti-HD drugs.

In preferred embodiments, the PEG-coated gold NP is conjugated to folate for intravenous delivery of folate to the HD brain. Au₃@PEG₁ₖ @FA NPs are successfully delivered to the cortex and striatum for improving motor functions in a subject with HD. The folate-loaded gold NP significantly reduce 8-OHdG level in the striatum and neuroinflammation.

In embodiments, without being bound to any theory, the NP inhibits p38α mitogen-activated protein kinase (MAPK) phosphorylation and pyruvate dehydrogenase kinase 1 (PDK1) in the HD brain, thus upregulating the oxidative phosphorylation pathway (downstream of PDK1) and suppressing pyroptosis-induced cell death (downstream of both kinases).

In embodiments, the administration of PEG-coated gold NPs, preferably Au₃@PEG₁ₖ@FA NPs, as a drug or drug carrier to the brain for treating HD in a subject, targets the cell death pathway pyroptosis without observable toxicity. In preferred embodiment, treatment leads to reduced pyroptosis and neuroinflammation in the striatum and cortex of the HD brain.

In embodiments, administration of the composition to a subject with HD results in significant NP clearance with limited systemic toxicity.

In embodiments, the composition has self-therapeutic capability for the treatment of HD.

In a third aspect, the subject invention discloses a method of synthesizing a gold nanoparticle for treating HD by conjugating gold nanoparticles with polyethylene glycol (PEG). The method further comprises conjugating a therapeutic agent with a linker to the PEG for delivering the therapeutic agent to its therapeutic targets.

In certain embodiments, the compositions of the instant invention may be formulated for parenteral administration e.g., by injection, for example, bolus injection, intravenous administration, intraperitoneal administration, or continuous infusion. In addition, the compositions may be presented in unit dose form in ampoules, pre-filled syringes, and small volume infusion or in multi-dose containers with or without an added preservative. The compositions may be in forms of suspensions, solutions, or emulsions in oily or aqueous vehicles. The composition may further contain formulation agents such as suspending, stabilizing and/or dispersing agents. In further embodiments, the active ingredients of the compositions according to the instant invention may be in powder form, obtained by aseptic isolation of sterile solid or by lyophilization from solution for constitution with a suitable vehicle, e.g., sterile, pyrogen-free water, before use.

The subject compositions can further comprise one or more pharmaceutically acceptable carriers, and/or excipients, and can be formulated into liquid forms, such as solutions for injections.

The term “pharmaceutically acceptable carrier” refers to an excipient that is a carrier or vehicle, such as a suspension aid, solubilizing aid, or aerosolization aid. Remington: The Science and Practice of Pharmacy, The University of the Sciences in Philadelphia, Editor, Lippincott, Williams, & Wilkins, Philadelphia, Pa., 21^{st} Edition (2005), incorporated herein by reference, describes additional compositions and formulations suitable for pharmaceutical delivery of one or more therapeutic compositions and/or pharmaceutical agents. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

The term “excipient,” as used in this disclosure, is an additive that is used in combination with the antigens. An excipient can be used, for example, to dilute an active agent, such as an antigen, and/or to modify properties of a pharmaceutical composition. Examples of excipients include, but are not limited to, water, magnesium stearate, stearic acid, vegetable stearin, sucrose, lactose, starches, hydroxypropyl cellulose, hydoxypropyl methylcellulose, xylitol, sorbitol, maltitol, gelatin, polyethyleneglycol (PEG), phosphate buffered saline (PBS), carboxy methyl cellulose, vitamin A, vitamin E, vitamin C, retinyl palmitate, selenium, cysteine, methionine, citric acid, methyl paraben, propyl paraben, sugar, silica, talc, magnesium carbonate, sodium starch glycolate, tartrazine, aspartame, benzalkonium chloride, sesame oil, propyl gallate, sodium metabisulphite, lanolin, polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, lactose, saline, phosphate buffered saline or organic buffers including but not limited to Tris (hydroxymethyl) aminomethane, and metal chelating agents. Metal chelating agents include, but are not limited to, organic compounds such as the amino acids glutamic acid and histidine, organic diacids such as malate, and polypeptides such as phytochelatin, biomolecules such as pyochelin, pyoverdine, enterobactin and Dopa, and synthetic chelates such as ethylenediaminetetraacetic acid (EDTA).

Other physiologically acceptable compounds include wetting agents, emulsifying agents, dispersing agents or preservatives that are particularly useful for inhibiting the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In one embodiment, the excipients are sterile and generally free of undesirable matter. These compositions can be sterilized by conventional, well-known sterilization techniques.

The concentration/amount of the compound or composition can vary widely, and are selected primarily based on activity of the active ingredient(s), body weight and the like in accordance with the particular mode of administration selected and the patient's needs. Concentrations, however, will typically be selected to provide dosages ranging from about 0.01 mg/kg/day to about 50 mg/kg/day and sometimes higher. Typical dosages range from about 0.1 mg/kg/day to about 25 mg/kg/day, preferably from about 0.1 mg/kg/day to about 10 mg/kg/day, more preferably from about 0.1 mg/kg/day to about 5 mg/kg/day, and most preferably from about 0.1 mg/kg/day to about 3.75 mg/kg/day. In preferred embodiments, the metal NP is administered to a human subject at a dosage of about 0.324 mg/kg/day. It will be appreciated that such dosages may be varied to optimize a therapeutic regimen in a particular subject.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, or more therapeutically effective doses of a composition comprising the compounds of the subject invention as described herein. In some embodiments, doses are administered over the course of 1 day, 2 days, 3 days, 4 days, 5 days, 6days, 7 days, 10 days, 14 days, 21 days, 30 days, or more than 30 days. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic assays known in the art. In some embodiments of the invention, the method comprises administration of the compounds at several time per day, including but not limiting to 2 times per day, 3 times per day, and 4 times per day. In some embodiments, the compound or composition is administered to a patient for about three to about five days.

In certain embodiments, the administration of at least one dose of the composition is repeated at least daily, biweekly, thrice-weekly, weekly, bimonthly, monthly, yearly or at least every about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, or longer.

In preferred embodiments, the administration of at least one dose of the composition is repeated at least daily, weekly, biweekly, thrice-weekly, bimonthly, or monthly. In more preferred embodiments, the administration of at least one dose of the composition is repeated at least weekly or monthly. In most preferred embodiments, the administration of at least one dose of the composition is repeated monthly.

In certain embodiments, the repeated administrations of at least one of dose of the composition occurs for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 52 weeks, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, or longer. In preferred embodiments, the administration of at least one dose of the composition is repeated every about 2 days to about 5 days for about 1 year, about 2 years, about 3 years, about 4 years, about 5 years, about 6 years, about 7 years, about 8 years, about 9 years, to about 10 years. In more preferred embodiments, the administration of at least one dose of the composition is repeated every about 2 days to about 5 days for about 1 years to about 5 years.

Further embodiments of the present invention including but not limited to the following experimental methods and results:

### Materials and Methods

Preparation of Auₓ@PEG_{y} NPs

Citrate-capped gold nanoparticles (AuNPs) of 3 nm in diameter are synthesized as described previously [10]. Briefly, 150 mL of freshly prepared sodium citrate (2.2 mM) containing 0.1 mL of tannic acid (2.5 m M) and 1 mL of potassium carbonate (K₂CO₃, 150 mM) is heated in a three-necked round bottom flask under vigorous stirring. When the temperature reached 70° C., 1 mL of HAuCl4 (25 mM) is injected and the reaction mixture is stirred for 5 min. The resultant 3 nm AuNP solution is stored at 4° C. Gold NPs of 13 nm in size are synthesized based on a modified Frens' method [11]. Briefly, after bringing 50 mL of HAuCl₄ (1 mM) to a boil, 5 mL of sodium citrate (1% w/v) is added under vigorous stirring and kept boiling for 15 min. The product is slowly cooled down to room temperature (RT). For both sizes, citrate-capped AuNPs are functionalized with thiolated PEG (mPEG₁ₖ-SH) at a concentration of 10 PEG molecules per nm² of NP surface under stirring overnight. To remove the free PEG strands, the NPs are washed three times by centrifugation at 2000× g for 5 min, using centrifugal filters with a membrane size cutoff of 50 kDa.

### Physicochemical Characterization of NPs

The concentration of AuNPs is determined by inductive coupled-plasma mass spectrometry (ICP-MS) with reference to a standard curve of known gold concentration (Au 197 isotope) in parts per billion (ppb). To obtain an equivalent mass of gold in the NP solution for injection into animals and to quantify the gold contents in tissues and cells, the present invention firstly converts the ICP-MS raw data from ppb to ug/L using the equation 1 ppb=1 μg/L, followed by multiplying the volume of dilute 2% nitric acid (HNO₃) that contains the digested tissues.

To characterize the morphology and measure the physical diameter of the entire PEG-coated gold NP (Au core+PEG), the NPs are negatively stained for TEM imaging. Briefly, 10 μL of aqueous NP solution are dropped onto a plasma-treated, formvar/carbon-coated copper grid (200 mesh) and left for 30 min. Then, the NP droplet is drawn off from the edge of the grid with filter paper. Next, EM Stainer solution is diluted with Nanopure water by 2 times, and 10 μL of the diluted solution is added to each TEM grid for another 10 min (EM Stainer is an electronic stain alternative to uranyl acetate.) After removing the EM Stainer solution, the grid is allowed to dry at RT for at least 4 h before visualization under TEM at a voltage of 100 kV, at a magnification of 30,000-50,000. At higher magnifications, the PEG shell does not have enough contrast to be imaged, especially for Au₃@PEG₁ₖ NPs with smaller gold core. At lower magnifications, the entire Au₃@PEG₁ₖ NPs appears too small to be imaged. Then, the length measuring function of the imaging software installed on-site at the TEM instrument is used to obtain an initial measurement of physical size. After this confirmatory step, more TEM images of a given NP sample are taken and analyzed using the Fiji image processing program as part of the ImageJ software. The physical diameters of the gold core and the PEG-coated gold NP are measured by manually drawing a straight line through the center of the NP from one end of the stained PEG shell circumference to another. Measurements are taken only where staining boundaries are clearly defined. At least 500 NPs for each NP type are counted.

The hydrodynamic diameters and zeta potential of NPs are measured by the dynamic light scattering (DLS) analyzer. Reported DLS data represent the values from three independent measurements. The DLS data quality is interpreted by analyzing the autocorrelation function and its fit, where the sum of square (SOS) threshold is set to be under 100. (Only measurements with an SOS value <100 are counted). As independent samples are used for different assays or experiments, a slight batch-to-batch variation is observed. To test colloidal stability, the NP solutions are mixed with equal volumes of fetal bovine albumin (FBS), and incubated at 37° C. for 24 h, followed by analysis using UV-vis spectrometry and DLS. Reported DLS values represent mean±SD from three independent measurements. Further, NPs are intravenously (i.v.) injected into the R6/2 mice mice (an established model of HD [12, 13]). After sacrificing the mice 24 h post-injection, ˜0.5 mL of blood plasma is collected from intracardiac puncture using an ethylenediaminetetraacetic acid (EDTA) tube and then centrifuged at 2000× g for 10 min. The AuNPs in the plasma supernatant are analyzed for their stability using DLS.

### Quantification of PEG Loading on the Gold NP

Method I: Indirect Measurement of Excess Thiolated PEG Strands Unattached to the Gold Core During Synthesis of Auₓ@PEG_{y} NP

Unmodified Aux NPs are functionalized with mPEG_{y}-SH at a concentration of 10 PEG molecules per nm² of NP surface under stirring overnight. The as-synthesized Auₓ@PEG_{y} NPs are collected without washing to retain the unbound mPEG_{y}-SH strands. To collect the free PEG strands, 10 mL of PEG standard and NPs are subjected to centrifugation at 2000× g for 5 min, using centrifugal filters with a membrane size cutoff of 50 kDa (Merck Millipore). PEG is detected as previously reported [14]. Briefly, 5 mL of the filtrates are collected and concentrated for 5 times by freeze drying. 20 μL of the concentrated PEG sample or standard is mixed with 100 μL of Ellman's assay buffer, 1 mM ethylenediaminetetraacetic acid (EDTA) in 0.1 M Na₂HPO₄ (pH 8). The resultant PEG sample or standard is mixed with 50 μL of Ellman's detection buffer [2 mg/mL Ellman's reagent 5,5-dithio-bis(2-nitrobenzoic acid) in the assay buffer]. After incubating at RT for 10 min, the absorbance is measured at 412 nm by the microplate reader (Molecular Devices). Subtraction of the amount of unbound PEG-SH strands from the known total amount of PEG-SH strands initially added to the unmodified gold cores will infer the loading of PEG on the gold core. PEG loading per NP is calculated by dividing the concentration of mPEG_{y}-SH attached to the gold core by the concentration of gold NPs (as determined by ICP-MS).

### Method II: Thermogravimetric Analysis (TGA)

A concentrated solution of Auₓ@PEG_{y} NP in Nanopure water is dried in an open alumina crucible at 80° C. for at least 2 h. This drying process continues until the dried NP material on the crucible is at least 1 mg in weight. The sample is analyzed by PerkinElmer TGA6, with a heating rate of 10° C./min from RT to 650° C. and nitrogen gas purging at a flow rate of 20 mL/min. The organic component of the NP (PEG) is found from the percent mass loss over the temperature range of 100-500° C., leaving behind gold (inorganic component) in the crucible after heating. Loading of PEG strands on the gold core (σ_{TGA}) is calculated using the following equation [15]:

$${σ}_{TGA} = \frac{\frac{wt {\%}_{shell}}{wt {\%}_{core}} {ρ}_{core} \frac{4}{3} π {r}_{core}^{3} {N}_{A}}{M W 4 π {r}_{core}^{3}}$$

σ_{TGA}: Loading of PEG strands on gold NP. Unit: Number of strands per surface area.
wt %ₛₕₑₗₗ: Relative mass of PEG determined from TGA data.
wt %_{gold}: Relative mass of gold determined from TGA data.

ρ_{core}: Density of gold=19.6 g/cm³. r_{core}: Radius of gold core.

N_{A}: Avogadro constant. MW: Molecular weight of PEG strand.

### In Vitro Cellular Uptake

Part 1: Cellular Uptake by ICP-MS

SK-N-MC cells are maintained in a 37° C. humidified cell culture incubator supplemented with 5% CO₂. Seeded in 24-well plates and grown till 70-80% confluence, the cells in each well are treated with 0.3 mL of transfection medium and incubated for 24 h. The transfection medium contains 2 μL of Lipofectamine 2000 and 1 μg of plasmid DNA (pEGFP-Htt1-550_{CAG89}) containing 89 repeating units of glutamine (Q89) expressing codons as reported previously, formulated in Dulbecco's modified eagle medium (DMEM) supplemented with 10% fetal bovine serum (FBS), 1% penicillin-streptomycin (P/S), which is the complete culture medium for the following study. Next, cells are incubated with 0.5 mL of 200 nM Au₃@PEG₁₀₀₀ NPs (in complete culture medium) for 2 h. After that, the cells are trypsinized with 200 μL of 0.25% trypsin-EDTA at 37° C. for 5 min, with 20 μL of the trypsinized cells used for staining with trypan blue and counting. The remaining 180 μL of trypsinized cells is dried in an oven at 60° C. overnight, digested with 0.5 mL of aqua regia [3:1 v/v ratio of 38% HCl and 68% HNO₃] for 1 h, and diluted to a 2% HCl, 2% HNO₃ solution with Nanopure water. Calibration standards of known gold concentration are prepared to convert counts of gold ion to known gold concentrations for ICP-MS measurements.

### Part 2: Cellular Uptake by Confocal Microscopy

In one embodiment, 35-mm confocal dishes are seeded or 24-well plates and grow in till 70-80% confluence, Q89-expressing SK-N-MC cells are incubated with 0.5 mL of 200 nM Au_{3@}PEG₁ₖ NPs for 24 h. After five PBS rinses (5 min each), the cells are fixed with 0.5 mL of 4% paraformaldehyde (PFA) overnight, rinsed with PBS for three times, stained with 0.5 mL of 4′,6-diamidino-2-phenylindole (DAPI) in PBS (5 μg/mL) for 30 min, washed with 1 mL of Nanopure water for five times, and finally stained using the LI Silver (LIS) Enhancement Kit (Invitrogen). Briefly, after mixing the silver enhancement solutions, Solution A (silver salt) and Solution B (initiator) at a 1:1 ratio right before use, the cells are incubated with 300 μL of the mixture in each well, incubated at RT for 30 min, rinsed with Nanopure water, and mounted on a glass slide. Confocal images are acquired in reflection mode with a 40× objective under 670 nm excitation [16].

### In Vitro Toxicity

Lactate dehydrogenase is a cytosolic enzyme which expresses in eukaryotic cells. This enzyme is released to cell culture medium when cell membrane is damaged upon cytotoxic insults. The LDH assay is a reliable colorimetric quantitative assay to measure the activity of the enzyme [17]. The enzyme activity has been shown to be directly proportional to the number of dead/damaged cells. In one embodiment, SK-N-MC cells are incubated with 200 nM of Au₃@PEG₁ₖ NPs in DMEM with 10% FBS. Lactate dehydrogenase (LDH) assay is performed 24 h post-transfection on SK-N-MC cells. The procedures are performed according to manufacturer's instructions.

Animal Models of Huntington's Disease (HD)

Transgenic B6.CBA-Tg(HDexon1)62Gpb/3J(22) mice (R6/2; Jackson Laboratory) were bred and maintained in a controlled environment with a temperature of 22±1° C. and a humidity range of 40-60%, following a 12-h dark/12-h light cycle at the Laboratory Animal Services Centre of The Chinese University of Hong Kong (CUHK). The transgenic mice were between 5 and 12 weeks old or transgenic female mice of 4-10 weeks old were used for experiments, whereas transgenic male mice were reserved for mating. Genotyping was performed based on 100 ng of genomic DNA (originally extracted from less than 2 mm of tail biopsies from each mouse). For the polymerase chain reaction (PCR; ABI PCR System 9700), the forward primer was 5′-CCGCTCAGGTTCTGCTTTTA-3′ (SEQ ID NO: 1) and the reverse primer was 5′-TGGAAGGACTTGAGGGACTC-3′ (SEQ ID NO: 2). For the internal positive control for genotyping, the forward primer was 5′-CTA GGCCACAGA ATTGAA AGATCT-3′ (SEQ ID NO: 3) and the reverse primer was 5′-GTAGGTGGAAATTCTAGCATCATCC-3′ (SEQ ID NO: 4). Non-transgenic littermates were used as control subjects. All procedures are approved by the CUHK Animal Experimentation Ethics Committee.

### In Vivo Organ-Level Distribution of NPs

### Part 1: Distribution as a Function of Disease Stage

R6/2 mice and healthy littermates of different ages (Week 4, 6, 8, or 10) received a single i.v. injection of 0.7 mg of Auₓ@PEG₁ₖ NPs (formulated in 0.1 mL of D5W). As Auₓ@PEG_{y} NPs have different sizes, the equivalent number of NPs per injected dose was different for each size, 1.76×10¹⁵ NPs for Au₃@PEG₁ₖ NP and 3.38×10¹³ NPs for Au₁₃@PEG₁ₖ NP. After sacrificing the mice 24 h post-injection, the organs were extracted and digested with 1 mL of aqua regia for 4 d at RT, unless otherwise stated. (Whole livers were digested with 3 mL of aqua regia). The digested samples were diluted to a 2% HCl, 2% HNO₃ solution with Nanopure water and then filtered with 0.1 μm acid resistant mixed cellulose ester (MCE; cellulose nitrate and cellulose acetate) filter using a 10 mL syringe. As R6/2 brains became progressively smaller with more severe neurodegeneration, the amount of gold in the brain was normalized to ug of gold per gram of tissue (μg/g) to eliminate bias due to the difference in tissue mass. On blood pharmacokinetics, Week 10 R6/2 mice were i.v. injected with 0.1 mL of 5% dextrose in water (D5W) containing 0.7 mg of Auₓ@PEG_{y} NPs. At various time points post-injection, 5 μL of blood was drawn by the tail vein using a 27 G needle and digested in 1 mL of aqua regia for ICP-MS measurements.

### Part 2: Distribution of NPs at Efficacy Evaluation

Healthy littermates and R6/2 mice received weekly i.v. injections of 0.1 mL of D5W containing 0.7 mg of Au₃@PEG₁ₖ NPs from the age of Week 6 to 10. At sacrifice (Week 11), tissues were harvested, weighted, and digested with aqua regia for ICP-MS measurements.

Part 3: Long-Term Clearance

The procedures largely followed Part 2 as described in the preceding paragraph, except that the mice were housed for 3 additional months and were sacrificed at the age of Week 24, the terminal age for sacrifice per LASEC guidance.

### In Vivo Intracranial Tissue-Level Distribution of NPs

### Part 1: Distribution as a Function of Disease Stage

R6/2 mice and healthy littermates of different ages (Week 4, 6, 8, or 10) received a single i.v. injection of 0.7 mg of Auₓ@PEG₁ₖ NPs (formulated in 0.1 mL of D5W). After sacrificing the mice 24 h post-injection, one half of the brain was dissected into five different parts [cortex (CTX), striatum (STR), hippocampus (HP), cerebellum (CB), and other regions (O)] as published²¹ and digested in 1 mL of aqua regia for ICP-MS analysis as above.

To visualize the intracranial location of gold NPs, we employed confocal immunofluorescence to label different cell types and confocal reflectance imaging to track the gold NP. Images of silver-enhanced tissue sections were obtained using a Leica SP8 confocal microscope and overlayed with other fluorescence channels. For 3D confocal images, a 63× objective was used. A z-stack series of images, obtained by focusing on the top and bottom of the tissue between two adjacent slides, was collected for 3D reconstruction by using the LAS X software (Leica). Identification of the CTX and STR regions was based on the Allen Brain Atlas (see worldwide website: mouse.brain-map.org/).

(i) Immunofluorescence: PBS-perfused brains were harvested, fixed in 10% formalin (3.7% w/v) for 24 h, and exchanged with 15% sucrose (w/v) in PBS at 4° C. until the brains sank, frozen in Shandon Cryomatrix frozen embedding medium (Thermo Fisher Scientific), and cut into sections of 40 um thick. After floating into PBS-containing 6 well plates, the cryosections were rinsed for three times (5 min each) and immersed in 1-1.5 mL of storage solution [30% (w/v) sucrose (TCI) and 30% (w/v), 30% (v/v) ethylene glycol (sigma) in 0.1 M phosphate buffer][18] for storage at −80° C. Using the floating technique, cryosections were brought to RT, washed with PBS for three times, blocked with 3% bovine serum albumin (BSA, Rockland) with 0.3% Triton X-100 (Thermo Fisher Scientific) in PBS for 1 h, and stained with primary antibodies in blocking buffer at 4° C. overnight. The primary antibodies include rat monoclonal against CD31 (Invitrogen, 14-0311-82; 1 μg/mL), guinea pig polyclonal against NeuN (Sigma-Aldrich, ABN90; 1:1000), rabbit monoclonal against glial fibrillary acidic protein (GFAP; abcam, ab68428; 0.1 μg/mL), rabbit polyclonal antibody against ionized calcium binding adaptor molecule 1 (iba1; Fujifilm, 019-19741; 1 μg/mL), and mouse monoclonal against HD exon-1 67Q aggregate (MW8; DSHB, AB_528297; 1.25 ng/ml). After rinses with PBS, the sections were stained with secondary antibodies at RT for 1 h: Alexa Fluor (AF) 488-conjugated goat anti-guinea pig (Invitrogen, A-11073; 5 μg/mL), AF546-conjugated goat anti-rabbit (Invitrogen, A-11035; 5 μg/mL), AF633-conjugated goat anti-mouse (Invitrogen, A-21052; 5 μg/mL), or AF647-conjugated goat anti-rat (Invitrogen, A-21247; 5 μg/mL). Slides were stained by 0.5 mL of 1 μg/mL DAPI for 10 min at RT, mounted with antifade mounting medium (Phygene, PH0429), and visualized under a confocal laser scanning microscope (TCS SP8, Leica) at identical imaging settings, in reflection mode for detecting AuNP reflectance. The excitation wavelengths of DAPI, AF488, AF546, AF633 are 405, 499, 561, 631 nm, respectively. The corresponding emission wavelengths are 415-470, 506-600, 571-630, and 640-740 nm, respectively.

(ii) Reflectance imaging: after three rinses with Nanopure water (5 min each) in 12 well plates, the cryosections were stained by the LI Silver (LIS) Enhancement Kit (Invitrogen). After mixing the silver enhancement solutions, Solution A (silver salt) and Solution B (initiator) at a 1:1 ratio right before use, the sections were incubated with 300 μL of the mixture in each well, incubated at RT for 30 min, rinsed with Nanopure water, and mounted on a glass slide. Confocal images were acquired in reflection mode with a 40× objective under 670 nm excitation⁷⁰.

### Part 2: Distribution of NPs at Efficacy Evaluation

Healthy littermates and R6/2 mice received weekly i.v. injections of 0.1 mL of D5W containing 0.7 mg of Au₃@PEG₁ₖ NPs from the age of Week 6 to 10. At sacrifice (Week 11), the brain was dissected for ICP-MS analysis and confocal imaging as stated in the preceding paragraph (Part 1).

### Part 3: Long-Term Clearance

The procedures largely followed Part 2 as described in the preceding paragraph, except that the mice were sacrificed at the age of Week 24.

### Entry of NPs to the HD Brain

### Part 1: Roles of Active Cellular Uptake

Week 10 R6/2 mice received an i.v. injection of 20 μL of dimethyl sulfoxide (DMSO; sigma that contains either 25 ug of filipin (Macklin, ˜1 mg/kg) or 750 μg of dynasore (Cayman Chemical); ˜30 mg/kg) via the tail vein. After 1 min, 0.1 mL of D5W containing 0.7 mg of Au₃@PEG₁ₖ NPs was i.v. injected into the other tail vein. After sacrificing the mice 24 h post-injection, half of the brain was dissected into tissue compartments and processed for ICP-MS measurements, while the other half of the brain was processed for immunofluorescence and reflectance imaging as described above.

### Part 2: Contribution of Passive Diffusion

Week 10 healthy littermates or R6/2 mice received an i.v. injection of 150 μL of 2% (w/v) Evans blue (Sigma). After sacrificing the mice 30 min post-injection, the mice were sacrificed and perfused with PBS via an intracardiac puncture using a 29G needle. Next, the brain was homogenized in 300 μL of lysis buffer containing 25% trichloroacetic acid (TCA; Sigma) in PBS. After incubating the brain lysate and a standard (2% Evans blue in lysis buffer) at 4° C. overnight, the samples were centrifuged at 1000× g at 4° C. for 30 min. The fluorescence of the supernatant (and standards) was measured by SpectraMax M3 (Molecular Devices) using the excitation and emission wavelengths of 620 nm and 680 nm, respectively. As R6/2 brains became progressively smaller with more severe neurodegeneration, the fluorescence in the brain was normalized to arbitrary units (AU) per gram of tissue to eliminate bias due to the difference in tissue mass.

### Part 3: Confocal Immunofluorescence

At sacrifice, the brain was excised from NP-treated R6/2 mice. Half of the brain was formalin-fixed, cryopreserved by sucrose, sectioned, and stained by DAPI. Tissue sections were stained by primary antibodies against iba1 and NeuN to indicate microglia and neurons respectively. For each section-containing slide, 2 pictures (290 μm×290 μm; 40× under a confocal microscope) were taken such that 4 pictures in both CTX and STR were counted for analysis of each mouse. 500 NeuN⁺ cells in both CTX and STR were selected, and NP-positive cells were counted. 200 iba1⁺ cells were selected for counting as they are less abundant than NeuN⁺ cells. Iba1⁺ area was measured by the ImageJ software. The total number of MW8⁺ aggregates in each image was counted and divided by the total number of NeuN⁺ cells. The average value of 4 pictures per mouse was displayed using a stacked bar chart with scatter plot points.

Part 4: Crossing the blood-CSF barrier

PBS-perfused brains were harvested, fixed in 10% formalin (3.7% w/v) for 24 h, and exchanged with 30% sucrose (w/v) in PBS at 4° C. until the brains sank. The perfused brains were frozen in Shandon Cryomatrix frozen embedding medium (Thermo Fisher Scientific) and cut into sagittal sections of 10 μm thick and mounted on Superfrost Plus™ Adhesion microscope slides (Thermo Scientific). After washing with PBS for 5 min, the sagittal sections were blocked in with 3% bovine serum albumin (BSA, Rockland) with 0.3% Triton X-100 (Thermo Fisher Scientific) in PBS for 1 h, and stained with primary antibodies in blocking buffer at 4° C. overnight. The primary antibodies include rabbit polyclonal against CD31 (Abcam, ab28364; 0.07 μg/mL) and mouse monoclonal against E-cadherin (BD Transduction, 610181; 1.25 μg/mL). After rinses with PBS, the sections were stained with secondary antibodies at RT for 2 h: AF 488-conjugated goat anti-mouse (Invitrogen, A-11029; 2 μg/mL) and AF 647-conjugated goat anti-rabbit (Invitrogen, A-21245; 2 μg/mL). Slides were stained by 1 μg/mL DAPI for 10 min at RT, mounted with antifade mounting medium (Phygene, PH0429), and visualized under a confocal laser scanning microscope at identical imaging settings, in reflection mode for detecting Au NP reflectance. The excitation wavelengths of DAPI, AF488, AF647 are 405, 499, 650 nm, respectively. The corresponding emission wavelengths are 415-470, 506-600 and 660-770 nm, respectively.

### Efficacy Evaluation by Behavioral Tests¹⁸

The day before the first evaluation on Week 6, healthy littermates and R6/2 mice were trained on the rotarod apparatus (Panlab) at a constant speed of 4 rpm for at least 1 min. On a given day on Weeks 6, 7, and 9, the mice took the open field test, rested for 1 h, took the rotarod test, rested for 1 h, and finally received an i.v. injection of 0.7 mg of Au₃@PEG₁ₖ NPs (formulated in 0.1 mL of D5W), all during the animal's light cycle phase. (On Weeks 8 and 10, the mice only received NP treatment but did not take the behavior test.) The negative treatment control entailed weekly i.v. injections of 70 kDa dextran (Sangon Biotech, A600375; formulated in 0.1 mL of D5W) from Weeks 6 to 10. The positive treatment control entailed daily i.p injections of tetrabenazine (Sigma; formulated in 0.1 mL of D5W containing 9% DMSO and 0.1 M citric acid⁷³) from Weeks 6 to 11. On Week 11, the mice took the last open field test and rotarod test before sacrifice. At sacrifice, after dissecting the brain into CTX and STR on an ice-cooled metal plate within 5 min, the CTX and STR tissues were weighed, snap frozen in liquid nitrogen, and stored at −80° C.

In a typical open field test, each mouse was placed in the center of an activity chamber (44×44×50 cm³) made from white, high-density, non-porous plastic equipped with a video camera (Panlab). The track and behavior of the mouse was recorded for 30 min, under low-stress and quiet conditions. Raw video data were analyzed using the Smart 3.0 Video Tracking Software (Panlab). Prolonged total distance travelled recorded within 30 min indicates the improved spontaneous locomotion of the animals due to NP treatment.

In a typical rotatod rest, the mice were placed onto the rotarod, one animal per lane. The rotation speed was initially 4 rpm and then linearly accelerated from 4 to 40 rpm over 5 min. Each mouse was tested five times in a 10-min interval with its staying time on the rotarod recorded, and the maximum and minimum staying time durations were removed before data analysis. A prolonged staying time on the rotarod indicates improved motor behavior due to NP treatment.

### Sample Size Calculation for Behavioral Studies

We used Dunnett's test to deduce the required size of each treatment group (N) [19].Dunnett's test is a multiple comparison procedure that compares the efficacy of each treatment group with the same control group. Here, we tested “HO: all treatment groups are equivalent to the control group” against “H1: there exists one group that is superior to the control group.” We compared the treatment groups and the control group in a way that (i) the chance of committing type 1 error is <5% and that (ii) our comparison is of power 80%. Dunnett's formalism states that p=√Nδ/σ, where p is the correlation coefficient that depends on N. There are three treatment groups and a control group (i.e., saline), so p is 4.3 [19]. If the superior treatment group gives an outcome (δ) of 1.5 SD (c) better than the control group, the required N is (4.3/1.5)²=8.22≈8.

### Efficacy Evaluation by Imaging and Molecular Assays

### Part 1: Confocal Immunofluorescence

At sacrifice, the brain was excised from NP-treated healthy littermates and R6/2 mice. Half of the brain was formalin-fixed, cryopreserved by sucrose, sectioned, and stained by DAPI. Tissue sections were stained by primary antibodies against MW8, iba1, NeuN that indicate HD exon-1 67Q aggregate, microglia, and neurons, respectively. For each section-containing slide, 2 pictures (290 μm×290 μm; 40× under a confocal microscope) were taken such that 4 pictures in both CTX and STR were counted for analysis of each mouse. Iba-1 positive area was measured by the ImageJ software. For the MW-8 positive aggregates, the total number of aggregates in each image was counted and divided by the total number of NeuN positive cells. The averaged value of 4 pictures per mouse was displayed using a stacked bar chart with scatter plot points.

### Part 2: Lipid Peroxidation

CTX and STR tissues were respectively homogenized (Biospec Products) in 300 or 600 μL of pre-cooled 20 mM Tris (pH 7.4) containing Pierce protease inhibitors (Thermo Fisher Scientific). After collecting the protein supernatants by centrifugation at 10,000× g for 5 min at 4° C., 100 μL of the supernatuant was used for determining the concentration of malonaldehyde (MDA) and 4-hydroxynonenal (HNE) per manufacturer's instructions of lipid peroxidation assay kit (Bioquochem, KB03002).

### Part 3: NAD⁺/NADH Ratio

After incubating the homogenized CTX and STR tissues on ice for 30 min for complete lysis, the protein supernatant was collected by centrifugation at 20,000× g for 10 min at 4° C. NAD⁺/NADH ratio was determined per manufacturer's instructions of NAD/NADH Assay Kit (Abcam, ab176723) by measuring the fluorescence at excitation and emission wavelengths of 540 and 590 nm, respectively, using SpectraMax M3 (Molecular Devices).

### Part 4: ATP Quantification

CTX and STR tissues were respectively homogenized in 300 or 600 μL of pre-cooled NET lysis buffer (20 mM Tris, 100 mM NaCl, 1 mM EDTA, 0.5% Triton X) containing Pierce protease inhibitors. After incubating the homogenized tissues on ice for 30 min for complete lysis, the protein supernatant was collected by centrifugation at 10,000× g for 5 min at 4° C. ATP standard ranging from 1 nM to 1 μM were prepared in NET lysis buffer as above. ATP determination kit (Invitrogen) was used to determine the ATP concentrations of the samples per manufacturer's instructions by measuring the luminescence using SpectraMax M3 (Molecular Devices).

### Part 5: Quantification of Caspase 3 Like Activity

CTX and STR tissues were respectively homogenized (Biospec Products) in 300 or 600 μL of pre-cooled 20 mM Tris (pH 7.4) containing Pierce protease inhibitors (Thermo Fisher Scientific). After collecting the protein supernatants by centrifugation at 10,000× g for 5 min at 4° C. and then diluted by 5 folds before adding to Caspase-3 Assay Kit (Thermo Fisher Scientific). Caspase 3 like activity was determined per manufacturer's instructions.

### Part 6: Western Blot Analysis

Initially, 5 μL of tissue lysate were diluted to 25 μL by water or different concentrations of serially diluted BSA (Pierce) were transferred into a 96-well plate. After adding 200 μL of freshly prepared BCA Protein Assay working reagent (Pierce) to each well, the samples were incubated at 37° C. for 30 min. Absorbance at 562 nm was measured using microplate reader SpectraMax M3 (Molecular Devices). Protein concentrations were calculated using BSA as a standard of calibration. Next, 20 μg of tissue lysate was electrophoresed through a 10% TGX denaturing polyacrylamide gel (BioRad) and transferred to a polyvinylidene difluoride membrane (BioRad) at 25 V for 10 min using a semi-dry transfer system (Power Blotter System, Invitrogen). After blocking in 5 mL of 5% BSA (Rockland) in Tris-buffered saline-Tween 20 (TBST) buffer for 1 h, the blots were incubated with 5 mL of primary antibodies diluted in TBST containing 5% BSA overnight at 4° C., including rabbit against phospho-MAPK 14 (p-p38α; Invitrogen, MA5-15177; 71 ng/mL), rabbit against MAPK 14 (p38α; Cell signaling; 23 ng/mL), rabbit against nucleotide-binding domain, leucine-rich-containing family, pyrin domain-containing-3 (NLRP-3; Invitrogen, SC06-23; 2 μg/mL), rabbit against beta-tubulin (B-tubulin; abcam, ab 108342; 46.5 ng/ml), rabbit against cleaved gasdermin D (GSDMD; Cell signaling, 36425, 18 ng/mL), rabbit against phospho-pyruvate dehydrogenase α1 (p-PDHα1, Cell signaling, 37115; 63 ng/mL), rabbit against pyruvate dehydrogenase (PDH, C54G1; Cell signaling, 3205; 15 ng/ml). After three PBS rinses, the blot was incubated with 5 mL of 1 μg/mL goat secondary antibody (conjugated with horse radish peroxidase) against rabbit (1706515; Bio-Rad) diluted in TBST containing 5% non-fat milk for 1 h. The membranes were treated with Clarity™ Western ECL Substrate (Bio-Rad) and the protein bands were visualized by the ChemiDoc Touch Imaging System (Bio-Rad).

### Part 7: ELISA

CTX and STR tissues were respectively homogenized in 300 or 600 μL of pre-cooled T-PERTM tissue protein extraction reagent (Thermo Fisher Scientific) containing Pierce protease inhibitors and phosphatase inhibitor cocktail (Bio-Platform) on ice. After incubating the homogenized tissues on ice for 30 min for complete lysis, the protein supernatant was collected by centrifugation at 10,000× g for 5 min at 4° C. and then diluted by 5 folds before adding to the ELISA kits. ELISA MAX™ Deluxe Sets mouse TNF-α (Biolegend), mouse IL-1β (Biolegend), mouse IL-4 (Biolegend), and mouse IL-6 (Biolegend) were used for ELISA quantification per manufacturer's instructions.

### In Vivo Toxicity

### Part 1: Long-Term Change in Behavior

Upon the completion of NP treatment from Weeks 6 to 10, both healthy littermates and R6/2 mice took the rotarod test every other week from Weeks 13 to 23 to monitor any adverse effects of NP treatment on animal behavior. Survival was monitored from Week 6 to Week 24.

### Part 2: Biochemical and Histological Assays

Following NP treatment on Week 10 or long-term monitoring on Week 23, R6/2 mice were sacrificed on Week 11 or 24, respectively. For histochemistry analysis, the brain and other major internal organs were fixed in 10% buffered formalin (3.7% w/v) for 24 h then stored in PBS (0.1M, pH 7.5) at 4° C. until tissue dehydration. Fixed tissues were dehydrated in ethanol, cleared in xylene, and embedded in paraffin blocks. Paraffin-embedded tissue sections (5 μm) were cut and mounted on Superfrost Plus™ Adhesion microscope slides (Thermo Fisher Scientific). Then, tissue sections were deparaffinized in xylene (5 min×3 times), rehydrated through a series of ethanol (100%, 90%, 70%; 3 min×2 times at each concentration) and Nanopure water (5 min×3 times), and stained with Harris hematoxylin (Sigma, HHS16) for 3 min and eosin (Sigma, 45260) for 30 s. To evaluate tissue morphology, bright-field images were taken with a Nikon Eclipse Ti microscope. Lastly, ˜1 mL of blood collected via an intracardiac puncture, was stored in plain tubes for biochemistry tests and in EDTA-coated tubes (Becton Dickinson) for hematology analysis. Blood samples were kept on mice and sent to PathLab (Hong Kong) for analysis on the same day.

### Therapeutic Role of Kinases

### Part 1: Kinase Panel Screening and IC₅₀

The effect of Au₃@PEG₁ₖ NPs on the activity of various kinases was assessed by SelectScreen Kinase Profiling Service (Thermo Fisher Scientific). The concentration of NP stock solution (20 μM; 100×) was verified by ICP-MS, followed by dialyzing NPs in water against absolute DMSO. Negative control 70 kDa dextran NPs were also prepared to be 20 μM (100×) in absolute DMSO. NP samples were sent for testing using the Z′LYTE biochemical assay. Briefly, Z′LYTE uses a fluorescence-based, coupled-enzyme format based on the differential sensitivity of phosphorylated and non-phosphorylated peptides to proteolytic cleavage. The NP sample was screened in 1% DMSO (final) in the well at a single concentration of 200 nM (1×). To calculate the IC₅₀ value of a screened high-performing kinase (with at least 85% inhibitory activity), the NP sample was subject to 10-point, 3-fold serial dilutions from the starting NP concentration of 200 nM (1×) for constructing the dose-response curve. Assays were conducted using the ATP concentration as indicated in the corresponding tables, depending on the format of detection. For “Direct Format” that operates through phosphorylation and activation of a synthetic peptide substrate for a given kinase, [ATP] was set at its apparent Kₘ value (Kₘ, apparent), as previously determined by the Z′-LYTE assay. For “Cascade Format” that operates through phosphorylation and activation of the inactive downstream kinase of a given kinase, [ATP] was set at 100 μM. Percent phosphorylation (% Pho) was determined with reference to the 0% phosphorylation (or 100% inhibition) control (which contains no ATP and therefore exhibits no kinase activity) and the 100% phosphorylation control (which contains the phosphorylated peptide as the same sequence as the peptide substrate). Control wells do not include any kinase inhibitors. Percent inhibition values were calculated by this equation: % Inhibition=[1−% PhoNP/% Pho0% inhibition ctrl]×100, whereby the 0% inhibition control contains the active kinase. IC₅₀ values were fitted from the dose-response curves based on model number 205 of XLfit from IDBS.

Part 2: Roles of p38α and Pyruvate Dehydrogenase 1 (PDK1)

To test the therapeutic role of selected kinases, R6/2 mice received daily i.p. injections of either (i) 0.1 mL of a vehicle (10% DMSO, 25% PEG₄₀₀ and 5% dextrose) containing an inhibitor of p38α called “p38-α MAPK-IN-1” (MCE) at a dose of 2.6 mg/kg, or (ii) 0.1 mL of D5W containing an inhibitor of PDK1, sodium dichloroacetate (DCA; Sigma, 347795) at a dose of 100mg/kg. On Weeks 6, 7, and 9, R6/2 mice underwent the open field test and rotarod test as described above. At the age of Week 11, the mice took the open field test and rotarod test again before sacrifice. The excised brain was dissected and processed for western blot analysis as above.

### Effect of NP Treatment on Protein Expression in the HD Brain

### Part 1: Protein Extraction and Digestion

Untreated and Au₃@PEG₁ₖ NP-treated R6/2 mice were sacrificed and perfused with 20 mL of PBS. Half of the brain was harvested, snap frozen in liquid nitrogen, stored at −80° C., and later sent to Biotech-Pack Scientific (Beijing) for protein extraction, protein digestion, Tandem Mass Tag (TMT) labeling, peptide fractionation, and analysis by liquid chromatography tandem mass spectrometry (LC-MS/MS; Thermo Fisher Scientific) and bioinformatics. Then, after adding 100 μL of RIPA lysis buffer to the sample, the sample was sonicated on ice, with 3 s of sonication and 3 s of pause, for a total of 5 min. After centrifuging the mixture at 10,000× g for 10 min at 4° C., the supernatant was transferred to a pre-chilled 1.5 mL EP tube. Next, 50 μg of the sample was transferred to a new EP tube and the volume was adjusted to 100 μL with 50 mM tetraethylammonium bromide (TEAB; Thermo Fisher Scientific). After adding 6 μL of 200 mM tris (2-carboxyethyl) phosphine (TCEP; Thermo Fisher Scientific), the sample was incubated at 56° C. for 1 h. After adding 6 μL of 375 mM iodoacetamide, the sample was incubated in the dark at RT for 30 min, mixed with six times the volume of pre-cooled (−20° C.) acetone, and frozen at −20° C. overnight to precipitate out the proteins. After centrifugation at 10,000× g at 4° C. for 10 min and discarding the acetone, the white precipitate (extracted protein) was air-dried for 2-3 min and resuspended in 50 μL of 50 mM TEAB. Lastly, to enzymatically digest the proteins, 1 μg of trypsin was added and the protein sample was incubated overnight at 37° C.

### Part 2: Tandem Mass Tag (TMT) Labeling and Peptide Fractionation

After removing from storage and equilibrating to RT, 20 μL of anhydrous acetonitrile (ACN; Fisher Chemical) was added to each vial of TMT labeling reagent (Thermo Fisher Scientific) and dissolved for 5 min. After adding the TMT solution to the digested protein solution from Part 1, the reaction was incubated at RT for 1 h and later quenched by adding 1 μL of 5% hydroxylamine with 15 min of incubation. Equal amounts of the labeled samples were combined in a new microcentrifuge tube and dried under vacuum at 45° C. For peptide fractionation, the protective white tip from the bottom of a chromatography column was removed, and the column was placed in a 2 mL sample tube, centrifuged at 5000× g for 2 min to pack the resin (with liquid discarded), rinsed twice with 300 μL of ACN (with centrifugation to discard the ACN), and equilibrated twice with 0.1% trifluoroacetic acid (TFA; Thermo Fisher Scientific). The labeled protein sample was dissolved in 300 μL of 0.1% TFA, loaded onto the column, and centrifuged at 3000× g for 2 min. To elute the protein sample, the column was added 300 μL of 5% ACN-0.1% TEA, centrifuged at 3000× g for 2 min to remove unreacted TMT. Repeat the elusion changing the 5% ACN to the following the sequence of 10%, 12.5%, 15%, 17.5%, 20%, 22.5%, 25% and 50% each and centrifuged at 3000× g for 2 min to collect the fractions. Each eluted sample was evaporated using a vacuum concentrator until dry.

### Part 3: LC-MS/MS and Data Analysis

LC-MS/MS entailed the use of a 50 μm i.d.×150 mm column, packed with Acclaim PepMap Reversed-Phase Liquid Chromatography (RPLC) C18, 1.9 μm, 100 Å. The mobile phases A and B were 0.1% formic acid in water and 0.1% formic acid, 80% ACN, respectively. The total flow rate was 300 nL/min, and a 120 min gradient involved 4-10% B, 5 min; 10-22% B, 80 min; 22-40% B, 25 min; 40-95% B, 1 min; and 95-95% B, 9 min. All MS and MS/MS spectra were acquired in data-dependent acquisition mode, and the full mass scan was acquired 350-1500 m/z with a resolution of 60,000. Raw data were analyzed based on the species of the samples using Proteome Discover 2.5. Identification of differentially expressed proteins (DEPs) was achieved by comparing the “Au₃@PEG₁ₖ NP-treated R6/2 mice” group to the “untreated R6/2” mice group. Proteins with a fold change ≥1.5 (P<0.05) were considered upregulated, while proteins with a fold change ≤1/1.5 (P<0.05) were considered downregulated. Pathway mapping of DEPs was performed using the Kyoto Encyclopedia of Genes and Genomes (KEGG) database (see worldwide website: genomc.jp/kegg/). Distribution of proteomics data in biological processes, cellular components, and molecular functions was obtained by mapping to the Gene Ontology database (see worldwide website: geneontology.org).

### Ex Vivo Skeletal Muscle and Neuromuscular Junction (NMJ) Functional Test

Week 11 R6/2 HD mice and age-matched healthy littermates were subject to skeletal muscle and NMJ functional tests ex vivo (800A, Aurora Scientific) [20-21]. Upon anaesthesia, the triceps surac-sciatic nerve complex was isolated from the right or left hindlimb of the mouse, ensuring intactness of the nerve terminals and NMJ structure. The surgical dissection to isolate the sciatic nerve from the surrounding tissue was performed in a dish with chilled (4° C.) simulated cerebrospinal fluid (CSF) buffer [22]. The isolated muscle-nerve complex was immersed in a synthetic interstitial fluid (SIF) buffer [123 mM NaCl, 3.5 mM KCl, 0.7 mM MgSO₄, 1.7 mM NaH₂PO₄, 2.0 mM CaCl₂, 9.5 mM NaC₆H₁₁O (sodium gluconate), 5.5 mM glucose, 7.5 mM sucrose, and 10 mM N-2-hydroxyethylpiperazine-N′-2-ethanesulfonic acid (HEPES); pH 7.4±0.05] [21], supplied with carboxygen (95% 02, 5% CO₂) for maintaining viability, and mounted onto the ex vivo apparatus between two parallel electrodes. The parallel electrodes stimulate muscle contraction directly, while the suction electrode induces muscle contraction indirectly by stimulating the sciatic nerve. The specific length at which the muscle fiber develops the largest isometric force, or a force of contraction without shortening, is regarded as the optimal length (Lₒ) of the muscle. A single pulse applied to muscle fiber results in a single contraction or “twitch”, while continuous or sustained contraction is termed “tetanic” contraction [23]. The decline in maximal force production in response to contractile stimuli is defined as muscle fatigue, induced by repeated tetanic stimuli to assess the skeletal muscle fatigability [24]. In this test, the L₀ was determined by stimulating muscle contraction at different muscle lengths with a small increment (0.2 mm), until the response twitch force was stabilized. At the Lo, the triceps surae muscle and sciatic nerve were stimulated by a single twitch stimulus with 1-min interval [(muscle stimulus: 300 mA, 0.2 ms pulse width; nerve stimulus: 5 mA, 0.8 ms pulse width; [20]]. After 1 min, the muscle and nerve were stimulated separately by a continuous tetanic stimulus with a 2-min interval (muscle stimulus: 300 mA, 300 ms duration, 0.2 ms pulse width, 50 Hz stimulation frequency; nerve stimulus: 5 mA, 300 ms duration, 0.8 ms pulse width, 50 Hz stimulation frequency). To assess fatigability, the triceps surae muscle was stimulated at 50 Hz at 0.7 s intervals for 100 consecutive cycles, a 15 min rest was allowed, and the sciatic nerve was stimulated with the same parameters. The intratetanic fatigue of muscle and NMJ were calculated from two consecutive tetanic pulse trainings, using the following equation [25]:

$$Intratetanic fatigue = \frac{{F}_{l p}}{{F}_{m}} \times 100 \%$$

Fₗₚ: force generated at the last pulse of stimulation in every 10 stimuli

Fₘ: the maximum force generated during the same pulse train in every 10 stimuli

### Skeletal Muscle Mass Measurement

Body compositional analysis of Week 11 R6/2 mice and age-matched healthy littermates was performed using dual-energy X-ray absorptiometry (DXA) imaging (KUBTEC PARAMETER 2D, Stratford, CT, US). After anaesthesia, mice were placed in prone position with four limbs extended away from the body. Imaging scans were taken, and lean mass percentage was calculated by the DIGIMUS software.

### Data Processing and Statistical Analysis

The Prism (GraphPad) and ImageJ software was used for data analysis and graph construction. Statistical analysis was indicated in the figure captions. Normality of sampling distribution of means was validated by the Shapiro-Wilk test. Homogeneity of variance was validated by the Bartlett's test. Results are considered significant when P<0.05.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Size- and Disease Stage-Dependent Delivery to the HD Brain

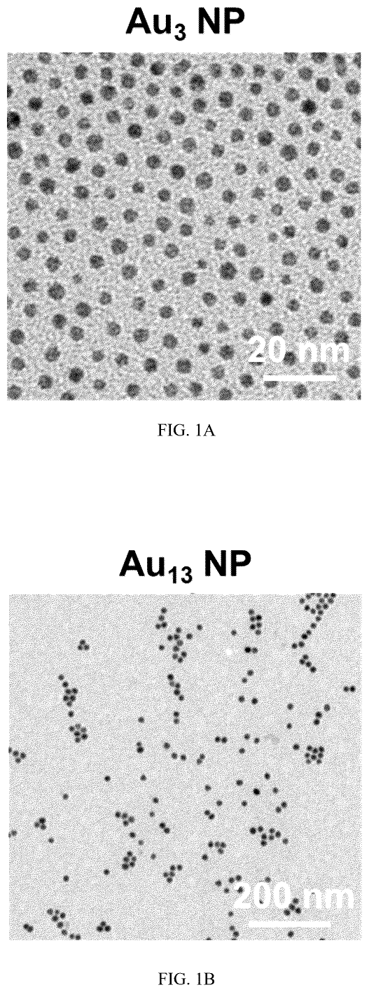

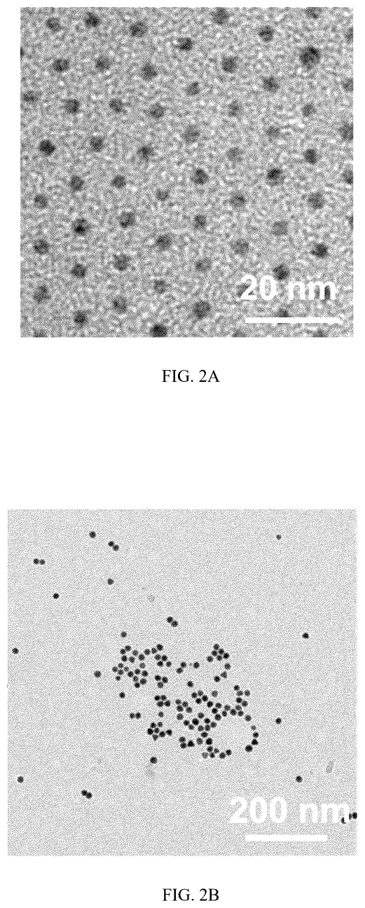

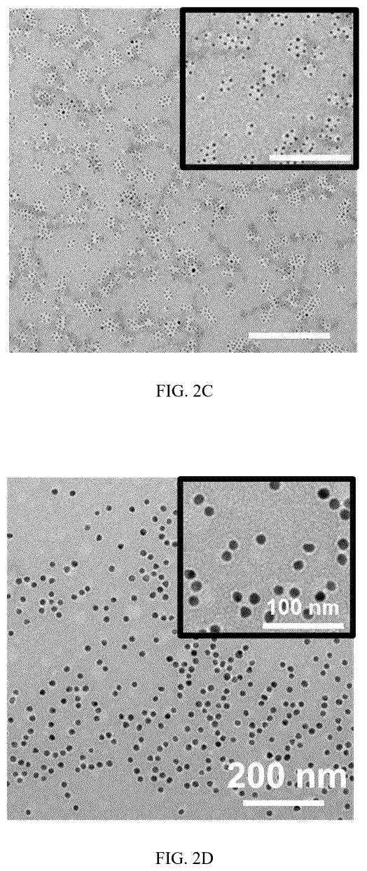

PEG-coated gold NPs (Aux@PEG1000 NPs) were prepared by reacting citrate-capped gold cores of two different diameters (x=3 or 13 in nm; FIGS. 1A-B) with excess thiolated PEG strands of 1000 Da in molecular weight. By negatively staining the NP for transmission electron microscopy, it was observed that the overall NP physical diameter (gold core plus PEG shell; FIGS. 2A-2B) matched its hydrodynamic size as measured by dynamic light scattering (˜11 nm for Au₃@PEG₁₀₀₀ NP and ˜23 nm for Au₁₃@PEG₁₀₀₀ NP; Table 1), implying a dense PEG coating [26].

TABLE 1
Physicochemical properties of Auₓ@PEG_{y} NPs.

| | | Physical | | | |
|---|---|---|---|---|---|
| | Physical | diameter of | Hydrodynamic | ζ potential | |
| | diameter | Au core + | diameter (nm) | (mV) in 1 | |
| | of Au core | PEG | in water | mM KCl | Polydispersity |
| Nanoparticles | (nm) | shell (nm)^{a} | at R ^{b} | at RT | index |
| Au₃ NP | 3.3 ± 0.6 | N. A. | 5.5 ± 0.6 | −27.9 ± 15.0 | 0.01 ± 0.01 |
| Au₁₃ NP | 12.7 ± 1.0 | N. A. | 17.0 ± 2.4 | −36.1 ± 0.8 | 0.05 ± 0.03 |
| Au₃@PEG₁ₖ NP | 3.0 ± 0.5 | 8.8 ± 1.0 | 10.7 ± 0.7 | −9.6 ± 0.5 | 0.03 ± 0.02 |
| Au₁₃@PEG₁ₖ NP | 11.6 ± 1.0 | 17.8 ± 1.1 | 22.5 ± 0.1 | −8.2 ± 1.3 | 0.05 ± 0.01 |
| Dextran 70 kDa | N. A. | N. A. | 10.2 ± 1.0 | −5.41 ± 0.4 | 0.14 ± 0.3 |

X = diameter of Au core (nm); y = molecular weight of PEG (Da); PDI = polydispersity index.
^{a}Physical diameters of the gold core and gold core + PEG shell are measured by TEM with negative staining by EM Stainer.
^{b} Hydrodynamic diameters, polydispersity indice (PDI), and zeta (ζ) potentials are measured by dynamic light scattering (DLS) at room temperature (RT).
All reported data represent mean ± SD from three independent measurements.

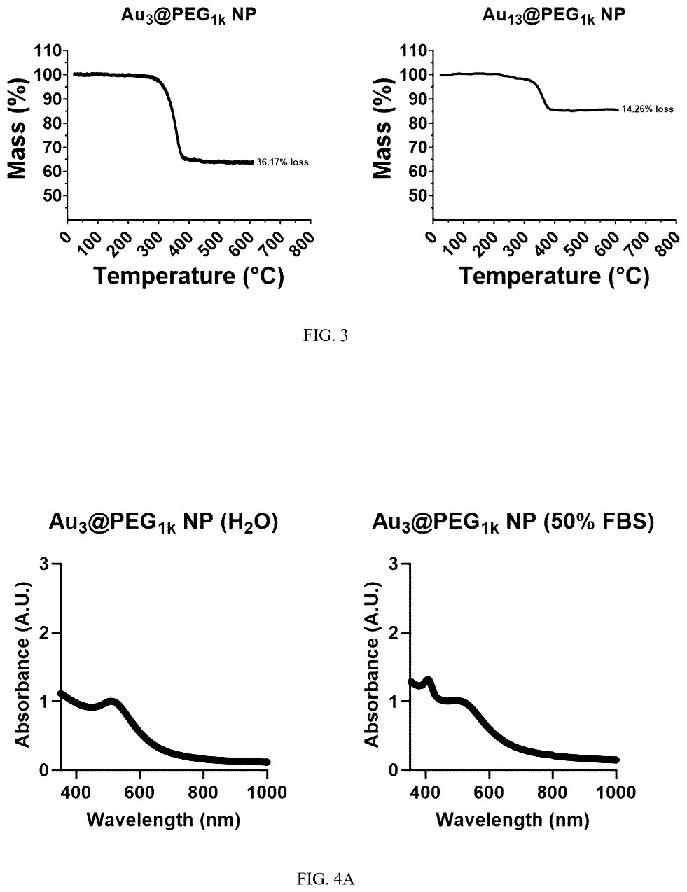

It was confirmed that the dense PEG coating (>3.5 PEG strands per nm² of gold surface [27]) using two methods, namely (i) indirect measurement of excess PEG strands unattached to the gold core during synthesis of Auₓ@PEG₁₀₀₀ NP by Ellman's test and (ii) direct measurement of the organic content of Auₓ@PEG₁₀₀₀ NP by thermogravimetric analysis (Table 2 and FIG. 3) [28].

TABLE 2
PEG loading on Auₓ@PEG_{y} NP using Ellman's assay (Indirect measurement)
and TGA (Direct measurement). All reported data represent mean ± SD from
three independent measurements. For TGA analysis, there were no technical replicates.

| | Ellman's assay | | | |
|---|---|---|---|---|
| | (Indirect measurement) | | TGA (Direct measurement) | |
| | Loading density | Loading density | Loading density | Loading density |
| Nanoparticles | (strands/NP) | (strands/nm²) | (strands/NP) | (strands/nm²) |
| Au₃@PEG₁ₖ NP | 182 ± 22 | 5.0 ± 0.6 | 135 | 3.7 |
| Au₁₃@PEG₁ₖ NP | 2229 ± 253 | 4.4 ± 0.5 | 2072 | 4.1 |

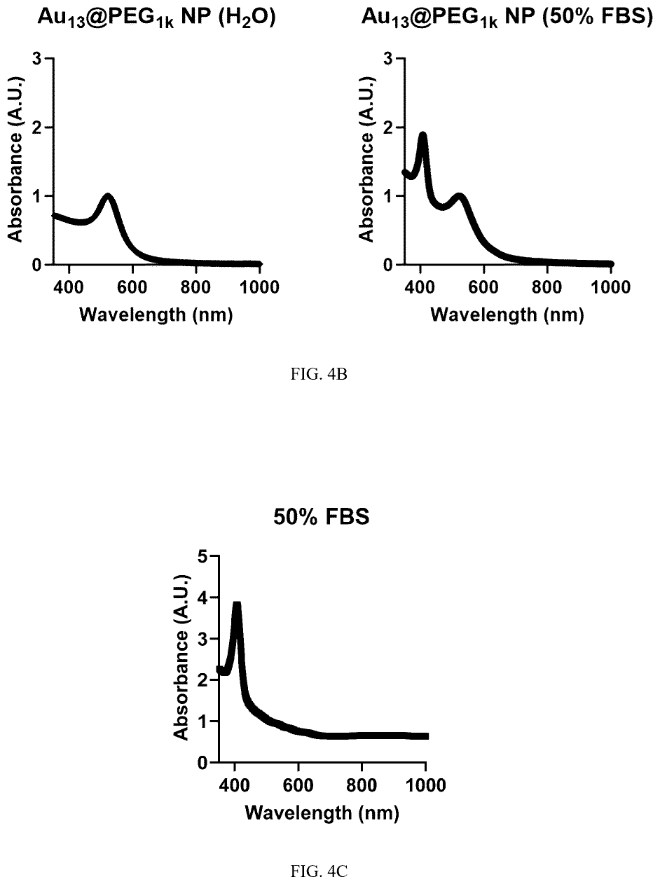

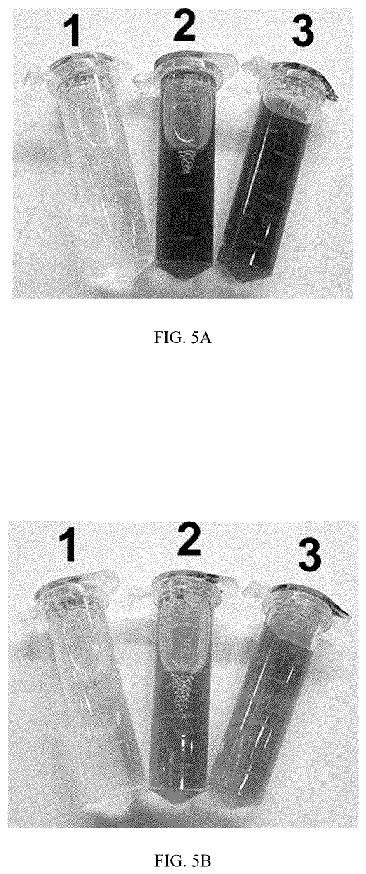

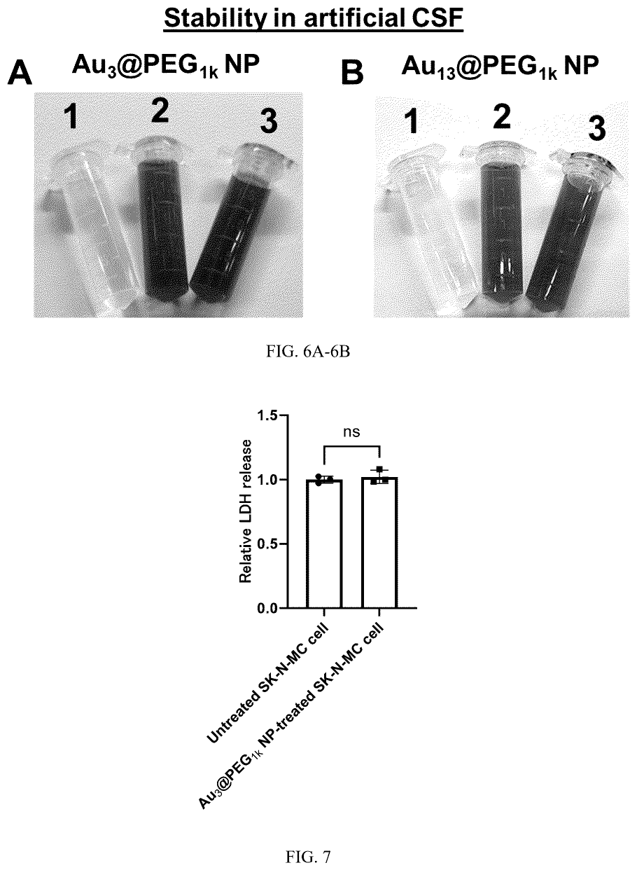

The PEG coating endowed the gold core with colloidal stability upon incubation in 50% fetal bovine serum and plasma of R6/2 HD mice at 37° C. for 24 h (Table 3, FIGS. 4A-4C, 5A-5B, and 6A-6B); with serum adsorption, the hydrodynamic size of both NPs became only slightly larger (14 and 26 nm). Au₃@PEG₁₀₀₀ NP did not induce toxicity to SK-N-MC neuroblastoma cells in vitro (FIG. 7).

TABLE 3
Hydrodynamic diameter (HD) of Auₓ@PEG_{y} NPs upon incubation in water
or 50% fetal bovine serum (FBS) at 37° C. for 24 h, or HD of Auₓ@PEG_{y}
NPs in plasma upon 24 h i.v. post-injection to Week 10 R6/2
HD mice. HDs were measured by dynamic light scattering.

| | Water | | 50% FBS | | R6/2 blood plasma | |
|---|---|---|---|---|---|---|
| NP | HD (nm) | PDI | HD (nm) | PDI | HD (nm) | PDI |
| Au₃@PEG₁ₖ | 10.7 ± 0.7 | 0.03 ± 0.02 | 11.7 ± 1.7 | 0.23 ± 0.06 | 13.8 ± 2.6 | 0.08 ± 0.04 |
| NP | | | | | | |
| Au₁₃@PEG₁ₖ | 22.5 ± 0.1 | 0.05 ± 0.03 | 23.25 ± 2.9 | 0.74 ± 0.01 | 26.3 ± 1.4 | 0.49 ± 0.14 |
| NP | | | | | | |

PDI = polydispersity index. All reported data represent mean ± SD from three independent measurements.

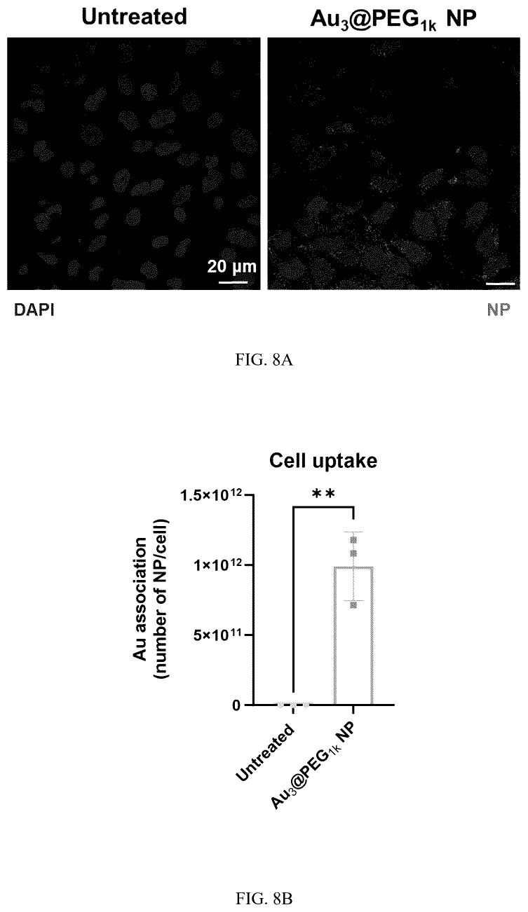

Furthermore, the uptake of Au₃@PEG₁₀₀₀ NP was verified by SK-N-MC cells that overexpressed 89 polyglutamine repeats (Q89), a model cell type of HD [29], by ICP-MS analysis and label-free confocal reflectance imaging (because gold NPs can reflect light) (FIGS. 8A-8B).

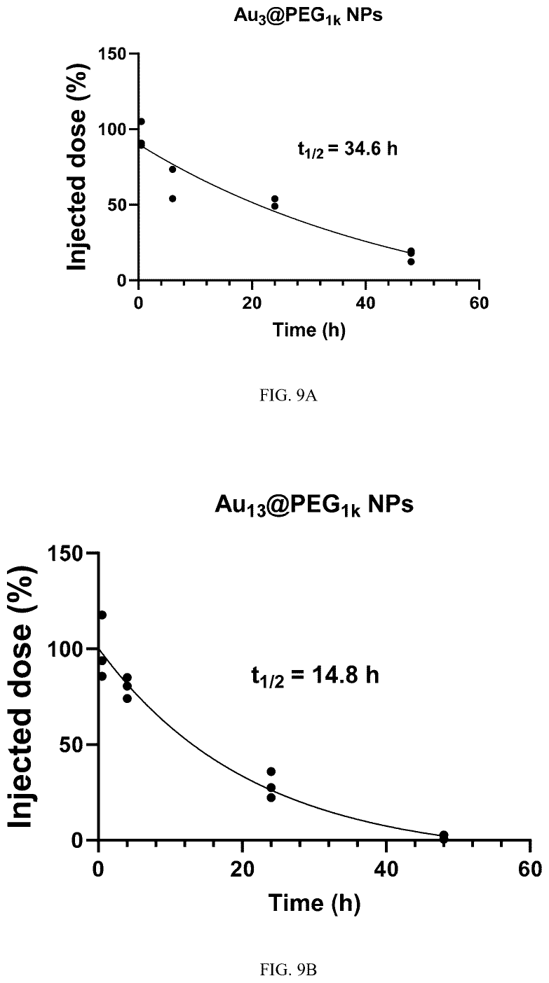

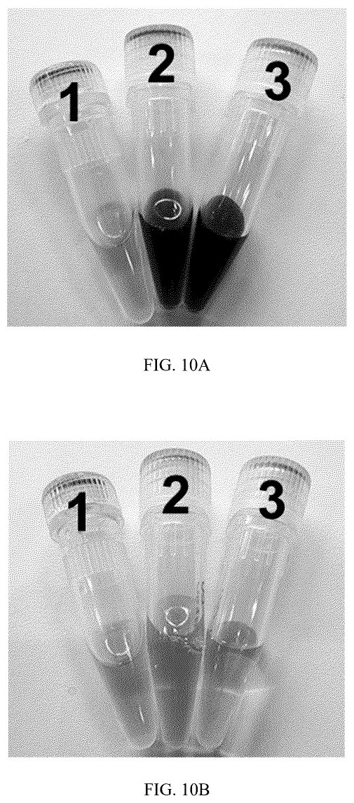

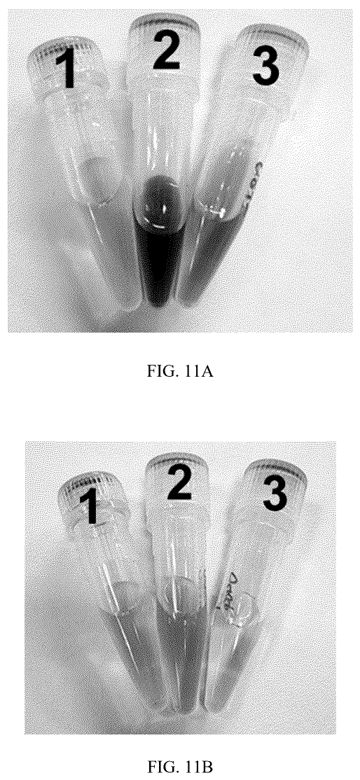

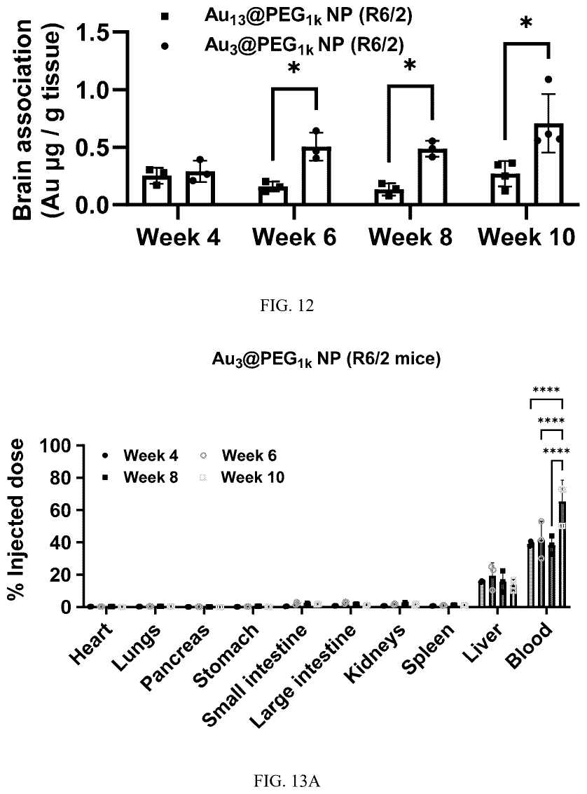

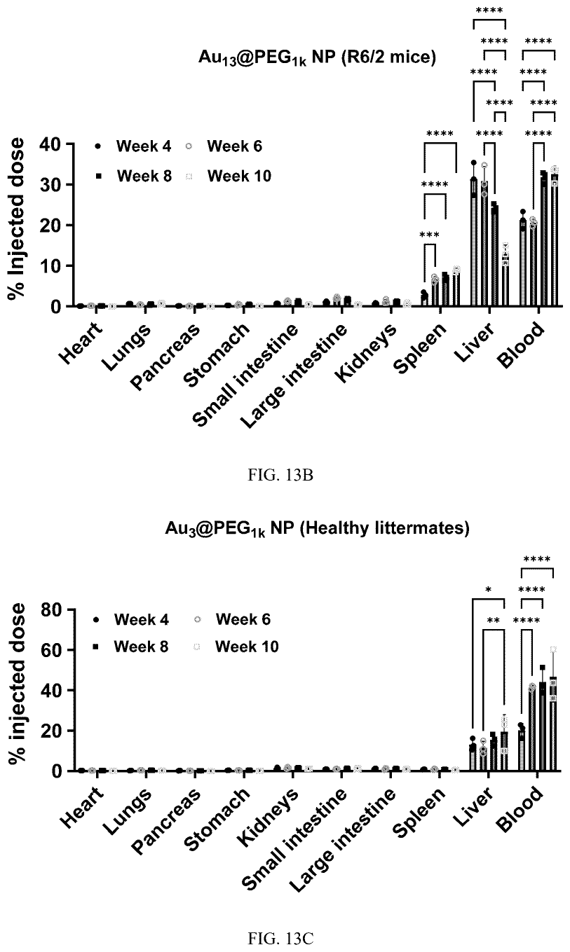

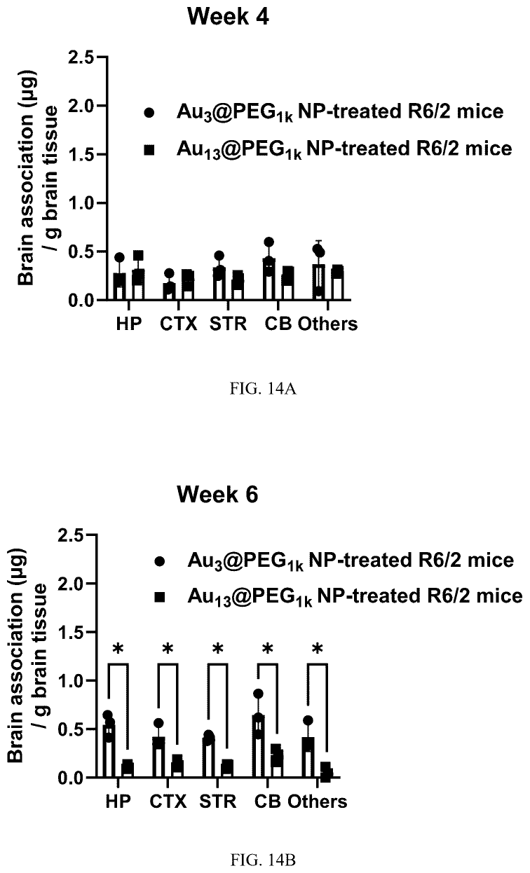

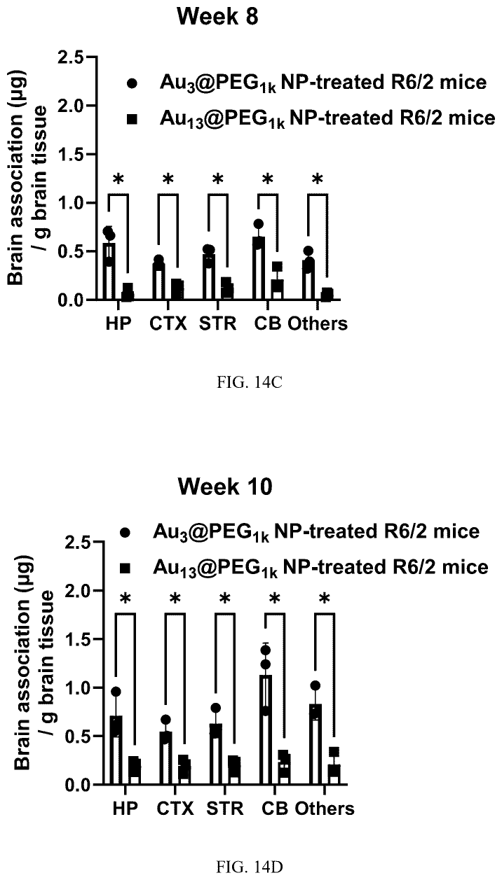

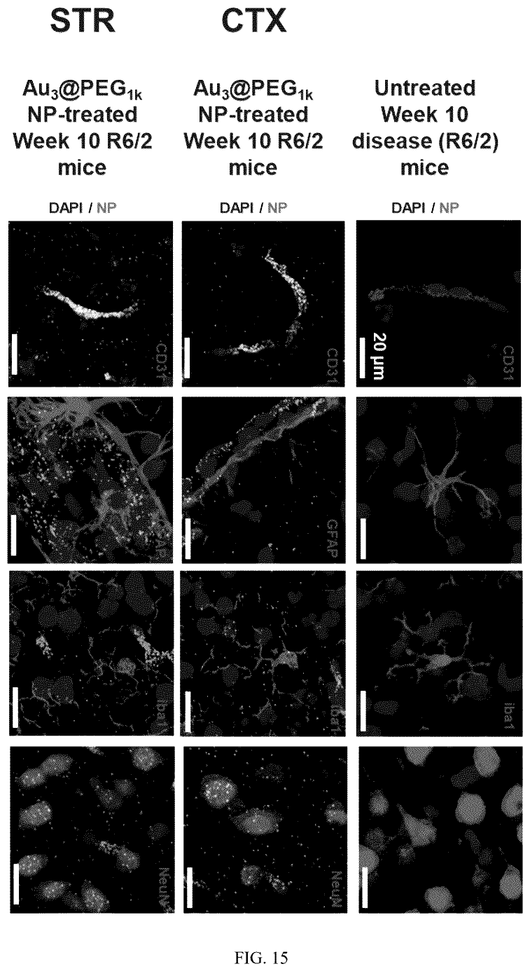

The shell of 1000-Da PEG strands endows the gold core with stability in blood [30] and promotes diffusion within the brain [31]. Upon i.v. injection into R6/2 transgenic mice the NP entered different cell types (neuron, glial cell, astrocyte, and endothelial cell) in the cortex and striatum; brain delivery depends on NP size and disease stage. The blood pharmacokinetics of both NPs following an i.v. injection into R6/2 mice at the age of Week 10, keeping the mass of gold injected constant at 700 μg per animal; this dose was used for i.v injection of siRNA-coated gold NPs for treating brain glioblastoma in mice [32]. Using inductively coupled plasma mass spectrometry (ICP-MS) to measure the gold contents in blood, a circulation half-life of ˜35 h and ˜15 h was detected for Au₃@PEG₁₀₀₀ and Au₁₃@PEG₁₀₀₀ NPs, respectively (FIGS. 9A-9B). Blood plasma isolated from the NP-injected mice revealed the red color characteristic of gold NPs 24 h post-injection (FIGS. 9-11), echoing the in vivo colloidal stability of NPs. Next, the organ-level distribution of both NPs as a function of HD stage 24 h post-injection were elucidated. ICP-MS analysis of the brain shows progressively higher accumulation of Au₃@PEG₁ₖ NP than Au₁₃@PEG₁ₖ NP from the age of Week 6 (onset of early-stage HD symptoms [33]) to Week 10 (near late-stage HD symptoms) (FIG. 12); we did not detect any noticeable difference in brain accumulation between the two NPs at the age of Week 4 (pre-symptomatic stage). Corroborating analysis of the liver showed more abundant accumulation of Au₁₃@PEG₁₀₀₀ NP than Au₃@PEG₁₀₀₀ NP (FIGS. 13A-13C). At the tissue level, there was more abundant accumulation of Au₃@PEG₁ₖ NPs in various compartments (hippocampus, cortex, striatum, cerebellum, and other regions) than Au₁₃@PEG₁ₖ NPs from Week 6 onwards (FIGS. 14A-14D). At the cellular level, confocal reflectance imaging of the cortex and striatum, the most severely affected compartments in HD [35], depicted strong localization of Au₃@PEG₁₀₀₀ NPs in endothelial cells [CD31; Manders' coefficient (MOC) ˜0.9] and some localization in astrocytes [glial fibrillary acidic protein (GFAP)], microglial cells [ionize calcium-binding adaptor molecule (iba) 1], and neurons (NeuN). Conversely, we captured limited reflectance signals of Au₁₃@PEG₁₀₀₀ NP in the same compartments (FIGS. 15-17). Summing up, the smaller Au₃@PEG₁₀₀₀ NP was more conducive to delivery to the HD brain and distribution to various tissue compartments and cell types.

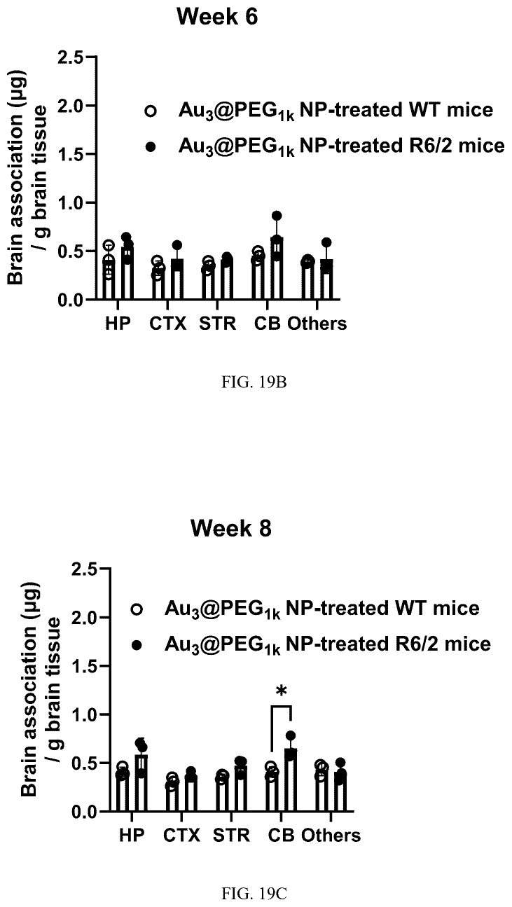

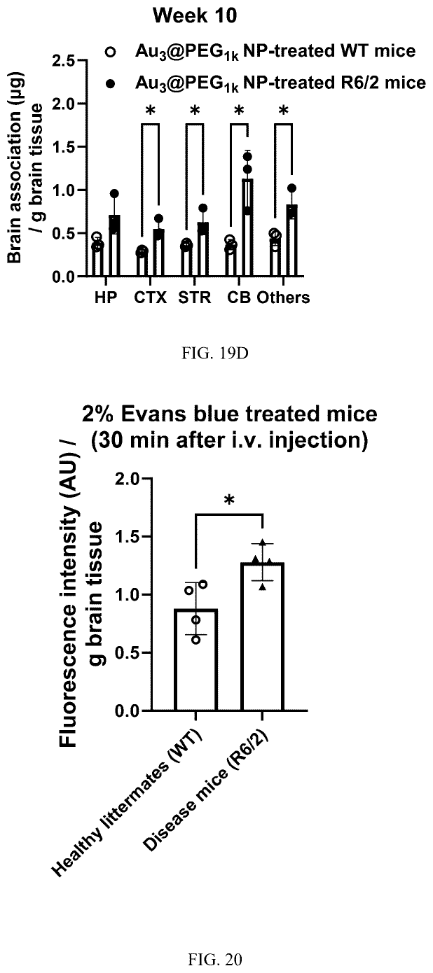

To decouple the effects of aging and disease stage on brain delivery, Au₃@PEG₁₀₀₀ NPs was injected to R6/2 HD mice and healthy wild-type (WT) littermates of the same age. Notably, ICP-MS analysis of the brain revealed progressively more abundant accumulation of NPs in R6/2 mice than healthy WT mice from Weeks 6 to 10 (FIG. 18), indicating more selective NP delivery to the HD brain as the disease deteriorated. At the tissue level, we detects enhanced delivery of NPs to the cerebellum of R6/2 mice than healthy WT mice at Week 8 and to other compartments (hippocampus, cortex, striatum, and cerebellum) at Week 10 (FIG. 19). Previous reports

documented BBB impairment or leakage in HD mouse models [36] and patients [37]. Here, upon i.v. injection of Evans Blue, a chemical dye that does not cross the healthy intact BBB, we detected mildly stronger fluorescence in the brain of Week 10 R6/2 mice than WT mice; direct visualization of blue color was infeasible (FIG. 20). This result implied modest dye deposition in the brain due to passive transport through BBB and entry to brain parenchymal cells, contrary to the visible blue color of Evans blue stain found in the brain of mice with glioblastoma (whose compromised BBB enabled the passage of larger ˜25-nm RNA NPs [32]). We then probed the role of active

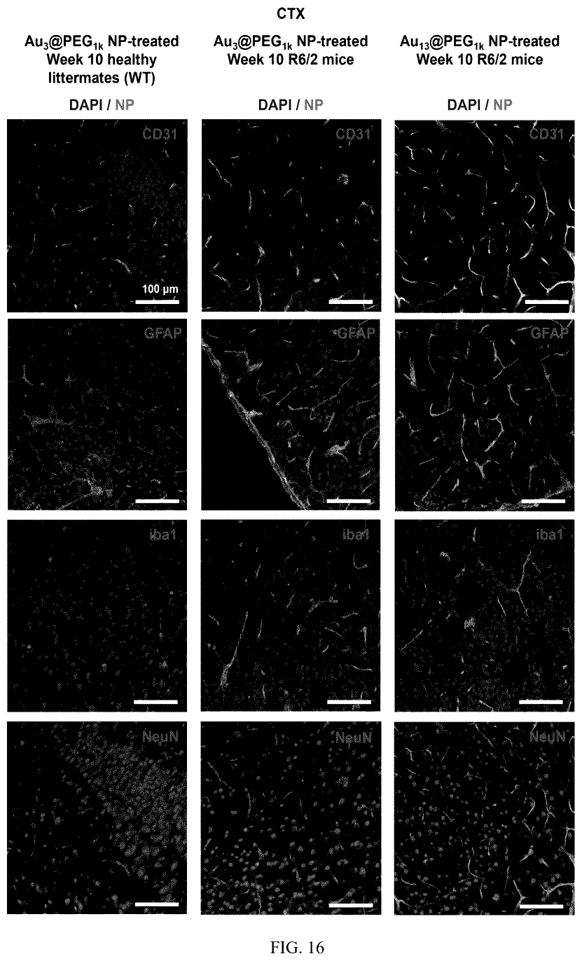

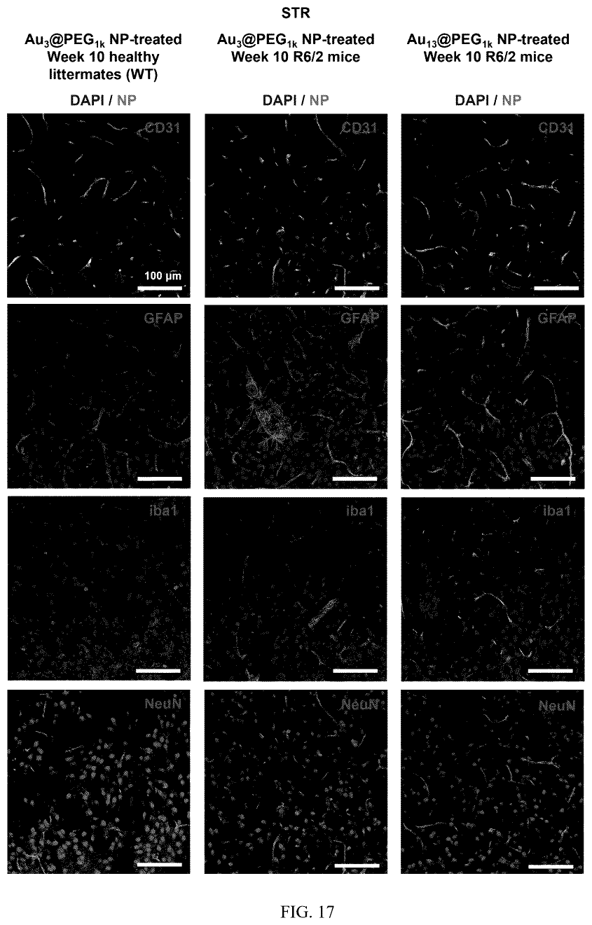

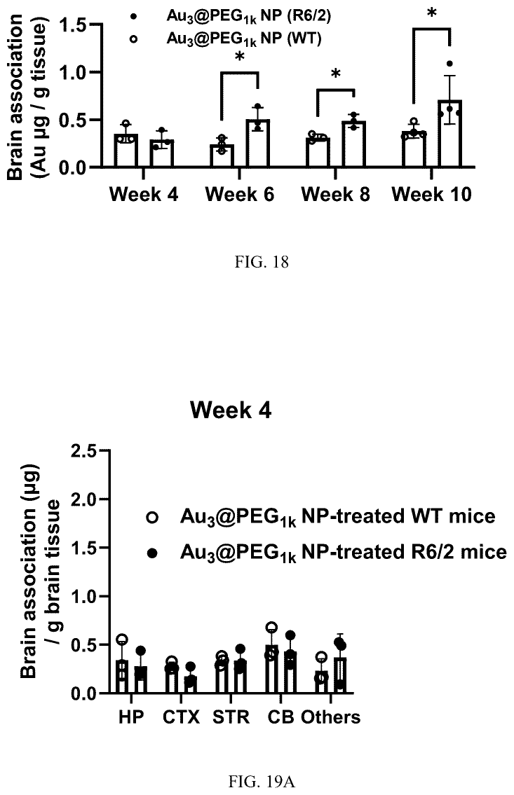

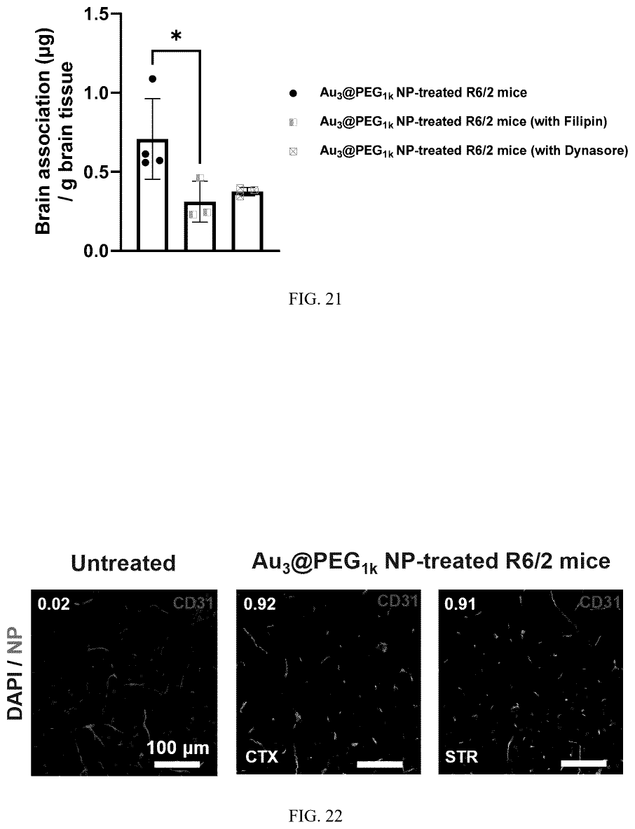

uptake of NPs by the brain in R6/2 mice. Pretreatment with filipin (a pharmacological blocker of caveolin-mediated uptake) or dynasore (a pharmacological blocker of dynamin-mediated uptake) attenuated the i.v. delivery of Au₃@PEG₁ₖ NPs to the brain (FIG. 21). Confocal immunofluorescence images verify strong colocalization of Au₃@PEG₁ₖ NPs with CD31-expressing brain endothelial cells in the cortex and striatum 24 h post-injection (FIGS. 16-17 and 22). Collectively, these data supported the notion of active transport through BBB endothelial cells and uptake by other parenchymal cell types, besides passive transport.

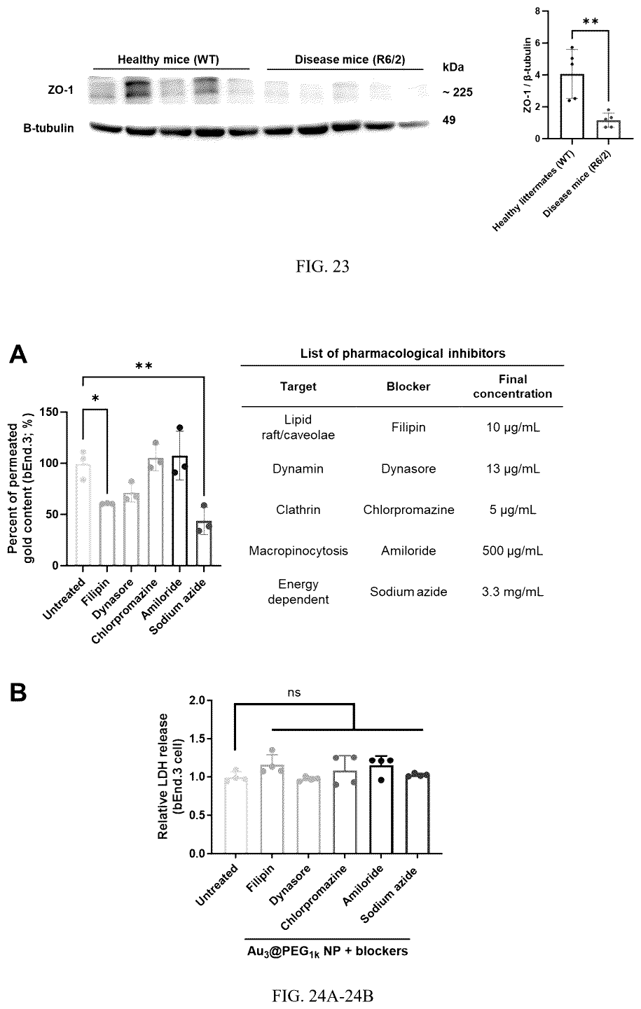

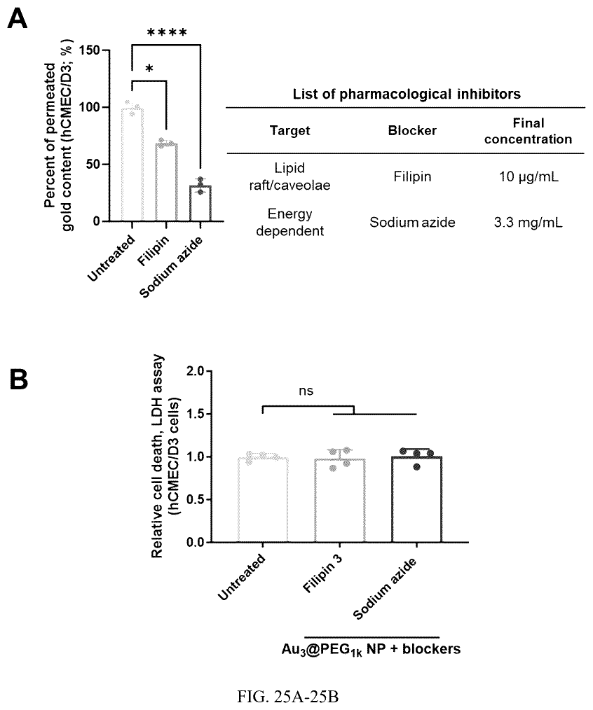

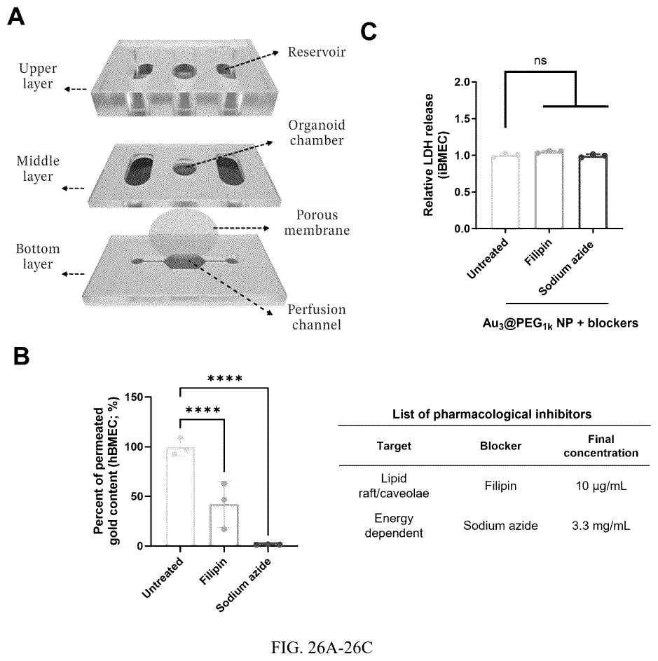

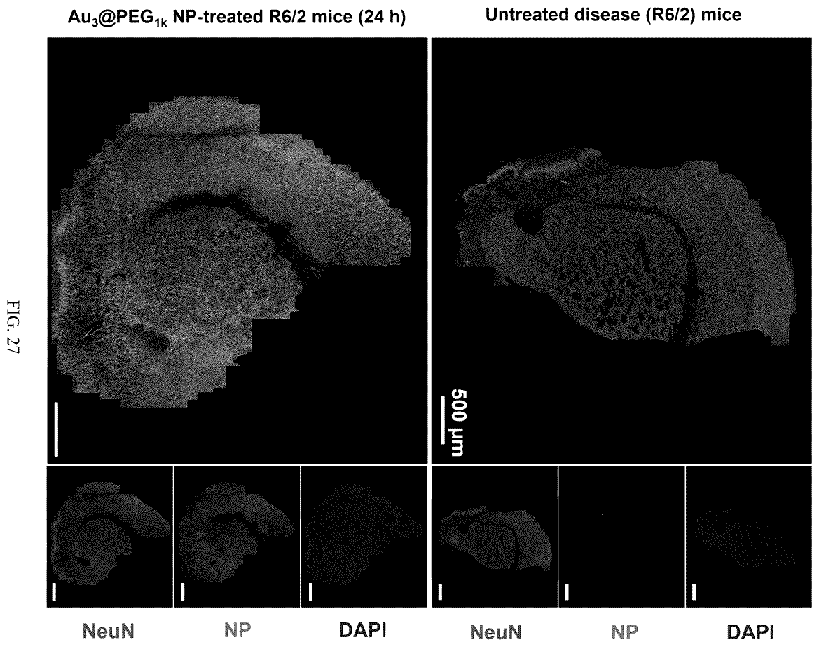

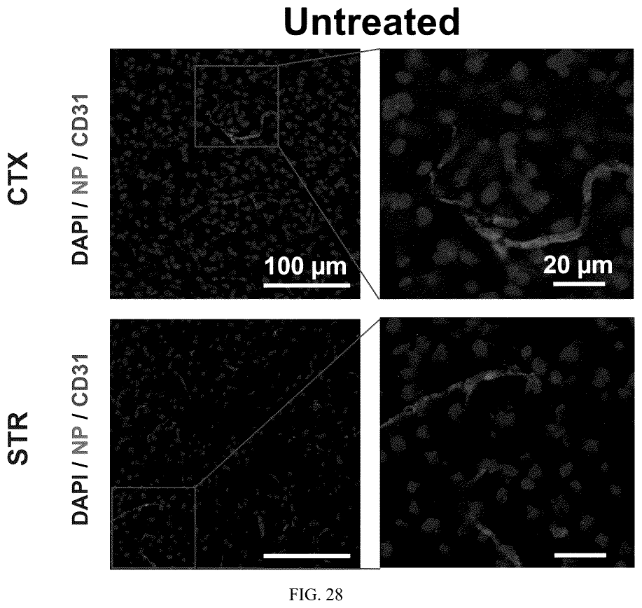

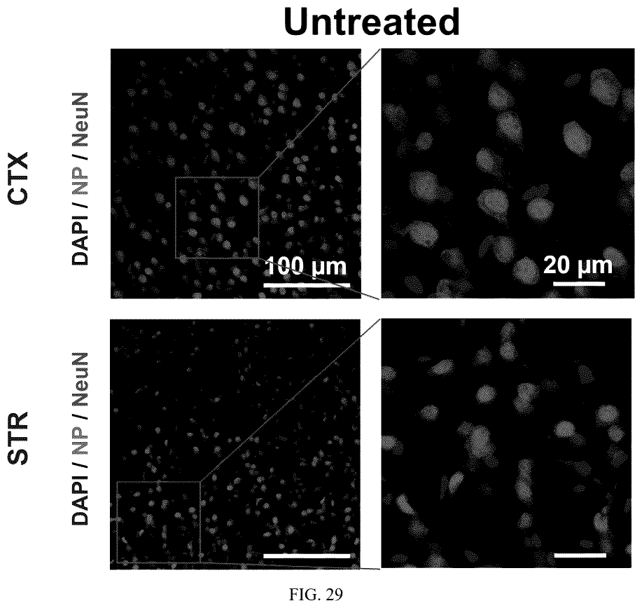

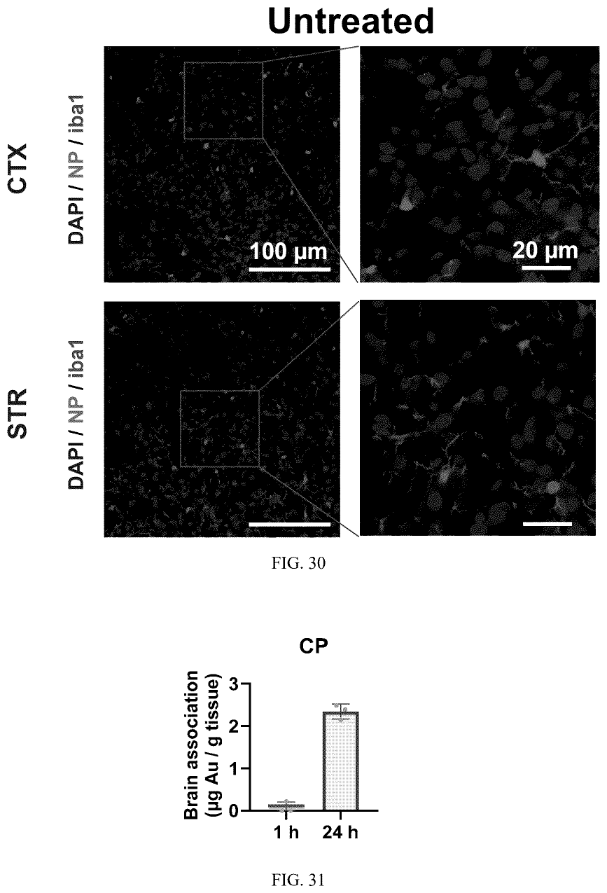

Western blot analysis revealed the downregulation of tight junction protein ZO-1 in the brain parenchyma of Week 10 R6/2 mice relative to healthy WT mice (FIG. 23). These results suggest some BBB leakiness to facilitate passive transport of Au₃@PEG₁ₖ NPs in R6/2 mice, contrary to the visible Evans blue color in the brain of mice with glioblastoma [whose compromised BBB enabled the passage of larger ˜25-nm RNA NPs [38]]. Next, we explored the possibility of active transport of Au₃@PEG₁ₖ NP across the BBB using three in vitro models, including basic Transwell setups seeded with mouse bEnd.3 cells and human hCMEC/D3 cells [both commonly used in the field of brain nanomedicine [39]] and an advanced fluid-flow microchip [40] seeded with human embryonic stem cell-induced brain microvascular endothelial cells (iBMECs) that gives a 6-fold higher transendothelial resistance than the two basic models. For the three BBB models of different species (human vs. mouse) and levels of tightness (bEnd.3 vs. iBMEC), pharmacological inhibition with filipin (caveolin-mediated endocytosis) [41] and sodium azide (energy-dependent cellular uptake) reduced NP permeation of the BBB by 31-58% and 56-98%, respectively (FIGS. 24-26), proof of the key role of active cellular transport in BBB penetration without the confounding factor of passive leakage through paracellular gaps. As no in vitro BBB model can fully recapitulate the in vivo characteristics of the brain, we further showed that treatment of Week 10 R6/2 mice with filipin attenuated brain accumulation, implying active NP uptake by brain cells in vivo; such pharmacological treatment did not induce cellular toxicity (FIGS. 24-26). A representative large, stitched confocal reflectance image of silver-enhanced coronal brain sections captured NPs throughout the tissue (including extracellular space and cells) 24 h post-injection (FIG. 27). Magnified confocal reflectance images depicted strong localization of NPs in endothelial cells [CD31; Manders' coefficient (MOC) ˜0.9] in both cortex and striatum, the most severely affected compartments in HD [42]. Tissues of uninjected mice had no silver stain (FIGS. 28-30). [During silver enhancement, silver ions will be catalytically reduced to metallic silver by gold NPs and deposited on their surface, forming amplified microscale silver clusters for visualization under a microscope [43].] This result suggests ample NP residency in the BBB as transient depots ([7], 44) 24 h post-injection before ultimate brain entry, rather than implying indefinite NP entrapment in the BBB. Confocal reflectance imaging detected moderate colocalization of NPs in neurons (NeuN) and activated microglia [ionized calcium-binding adaptor molecule (iba) 1], with MOC of ˜0.4. As Au₃@PEG₁ₖ NP does not have targeting ligands for a specific brain cell type, we do not expect a tendency toward neuronal accumulation.

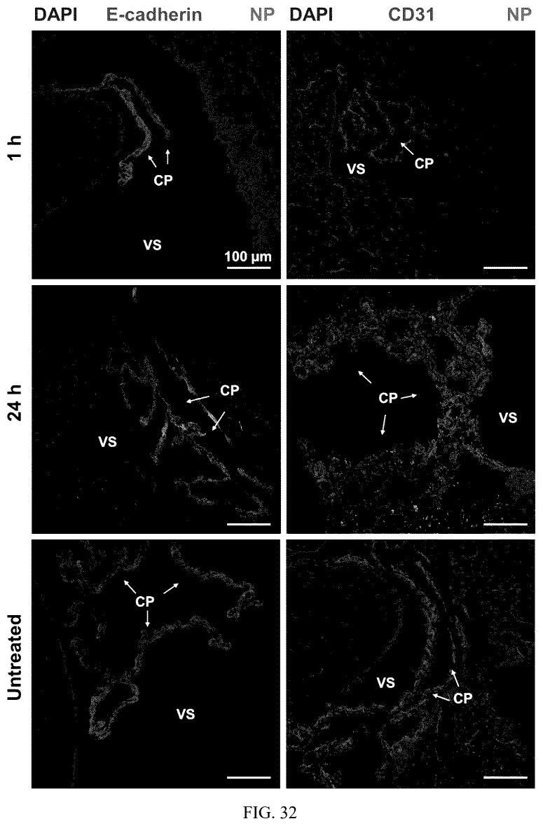

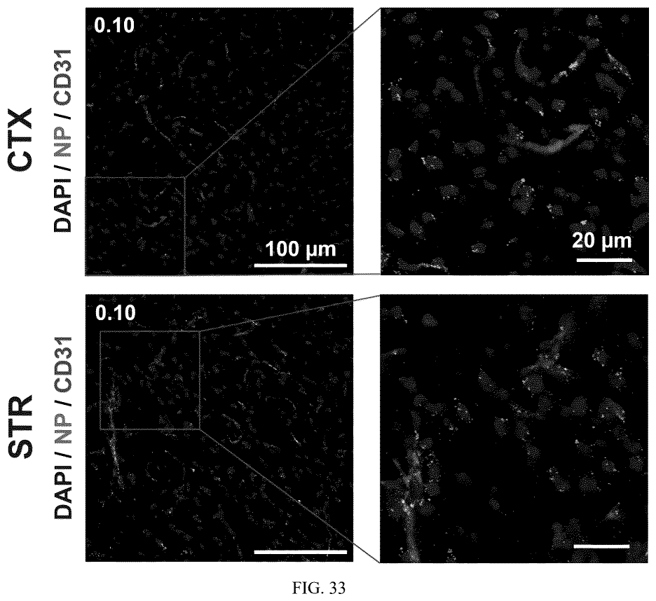

It is challenging to claim that Au₃@PEG₁ₖ NP solely enters the brain via the BBB because it may also undergo filtration at the blood-CSF barrier [45], noting that similarly sized proteins can cross the blood-CSF barrier [46]. ICP-MS measurements revealed detectable gold levels in the choroid plexus (site of the blood-CSF barrier) 24 h post-injection of Au₃@PEG₁ₖ NP into Week 10 R6/2 HD mice (FIG. 31). Confocal imaging captured gold reflectance signals in the choroid plexus (with its epithelial cells immunostained by E-cadherin) and adjacent BBB (with its endothelial cells stained by CD31) (FIG. 32). Similarly, the data only proved the existence of NPs in the choroid plexus 24 h post-injection, while not suggesting indefinite confinement in the blood-CSF barrier.

### EXAMPLE 2

### Entry to Neurons and Alleviation of HD Without Observable Toxicity

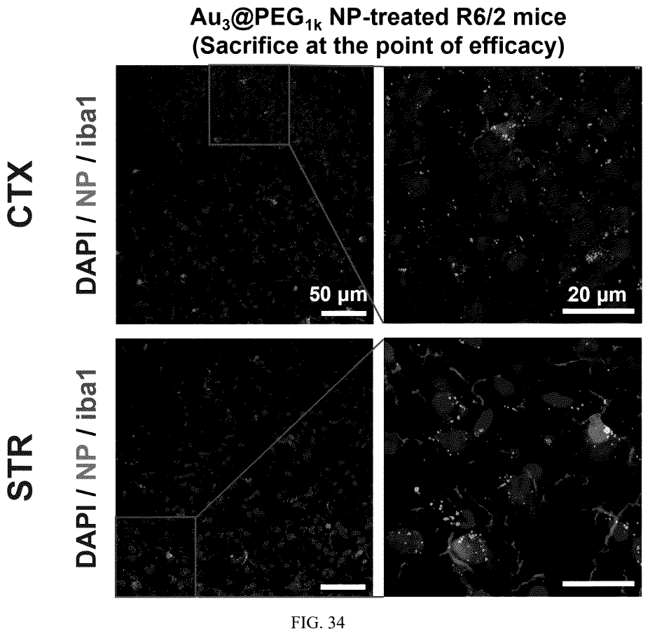

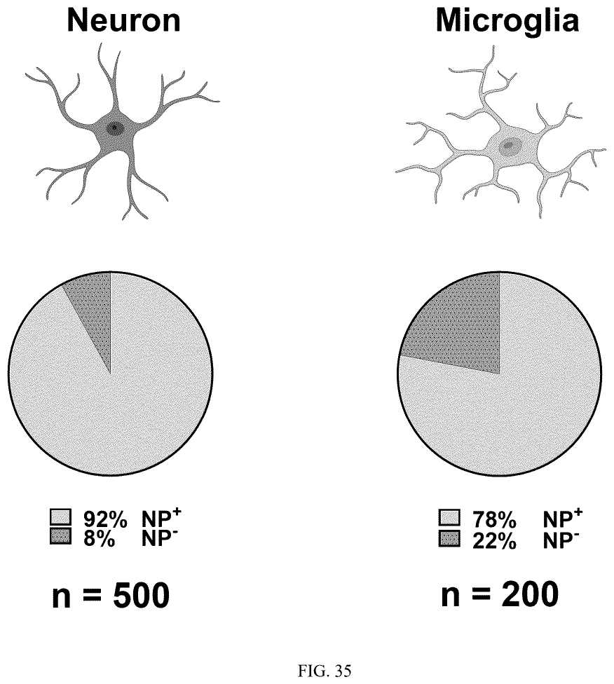

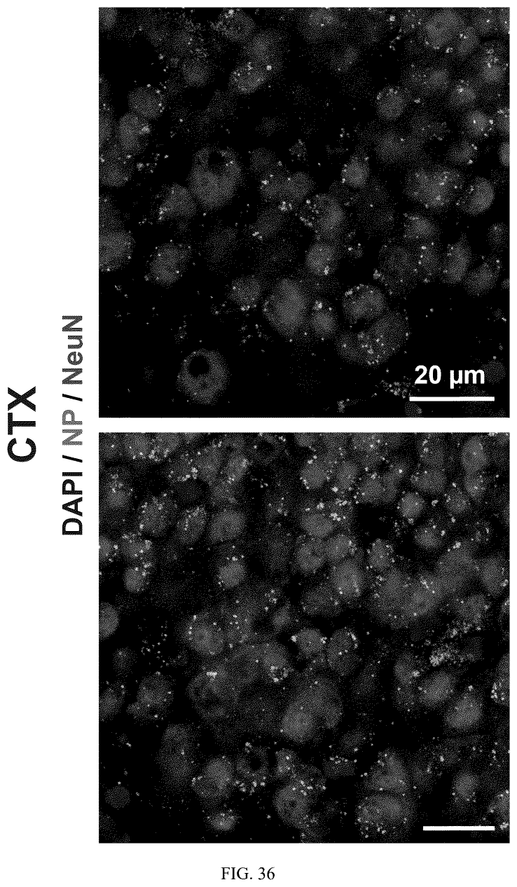

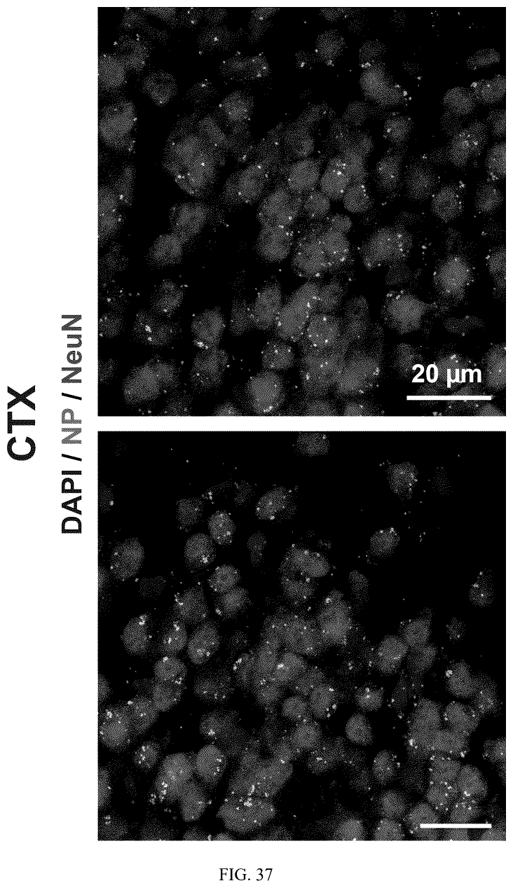

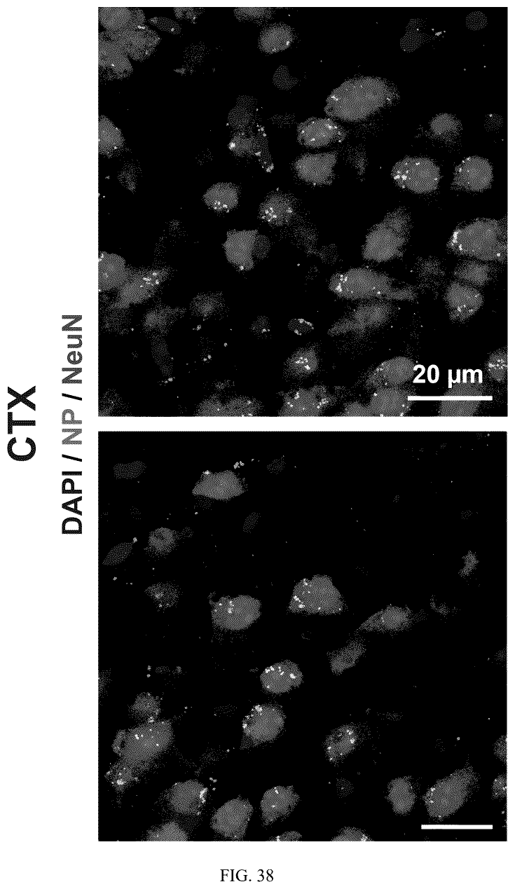

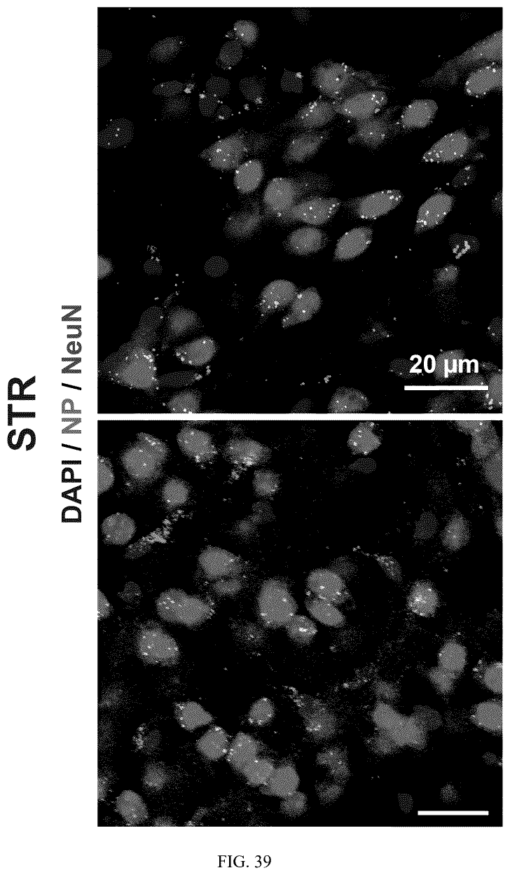

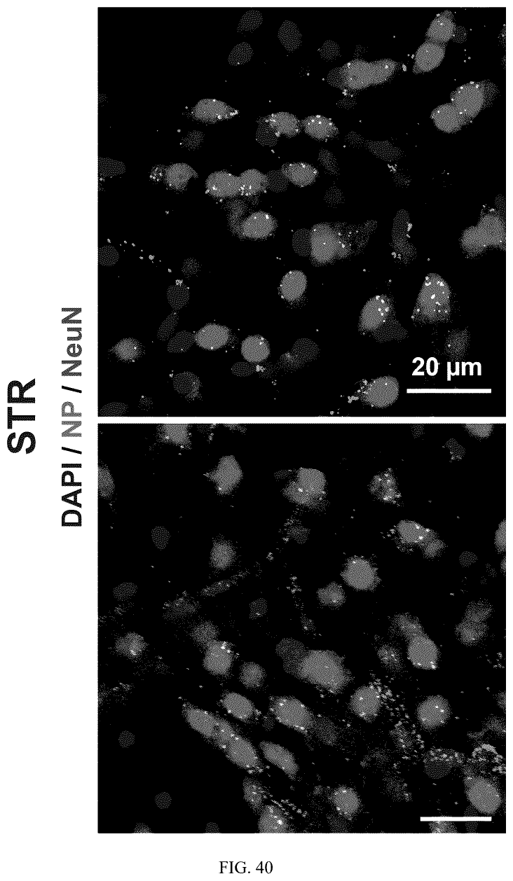

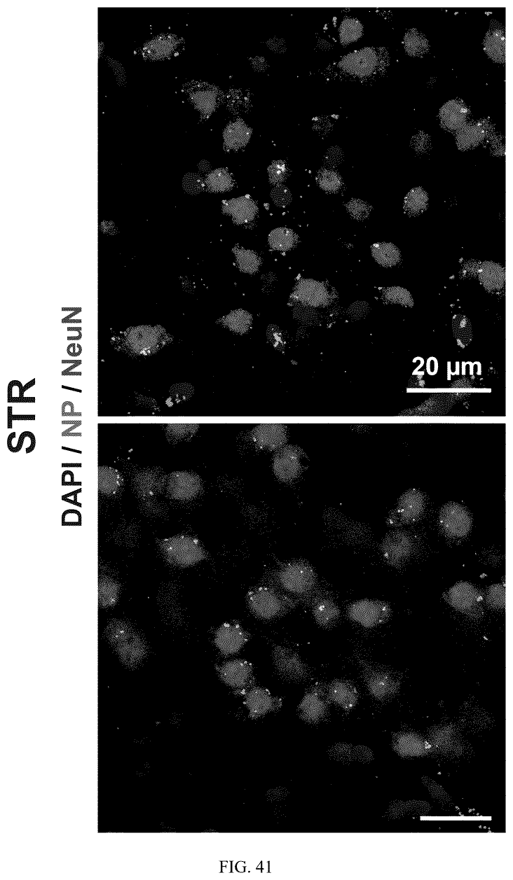

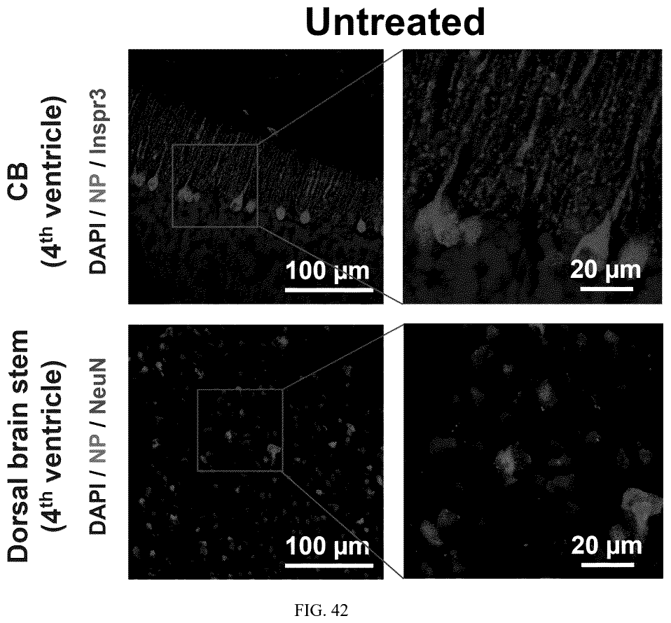

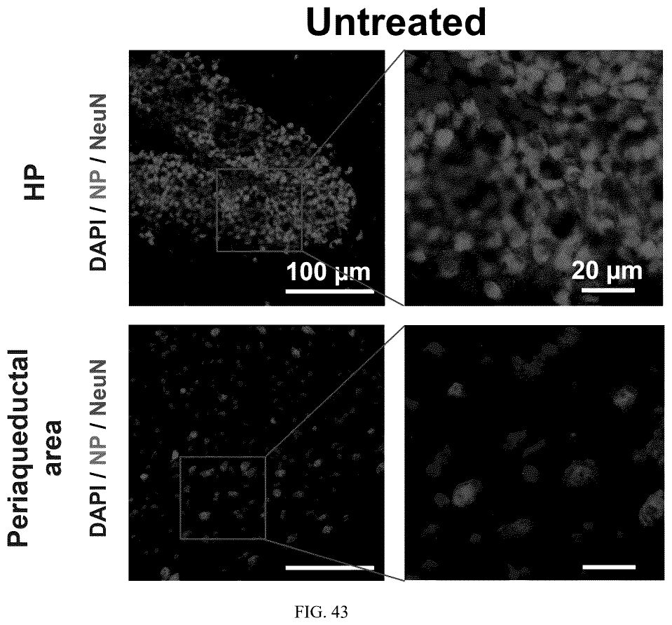

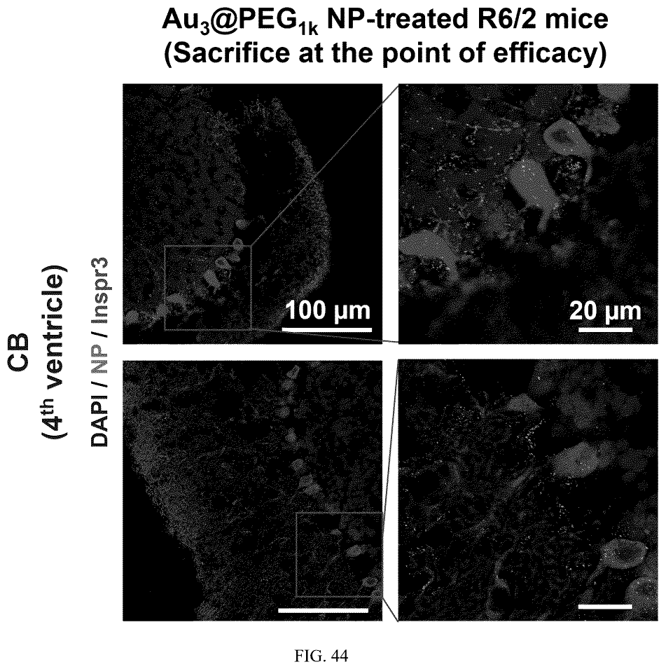

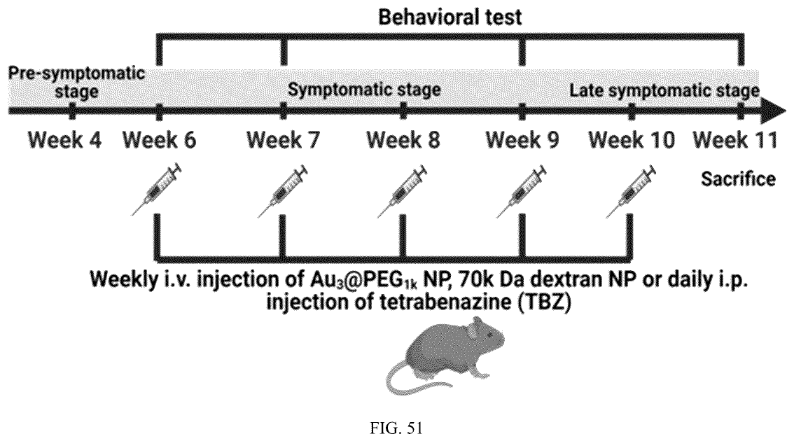

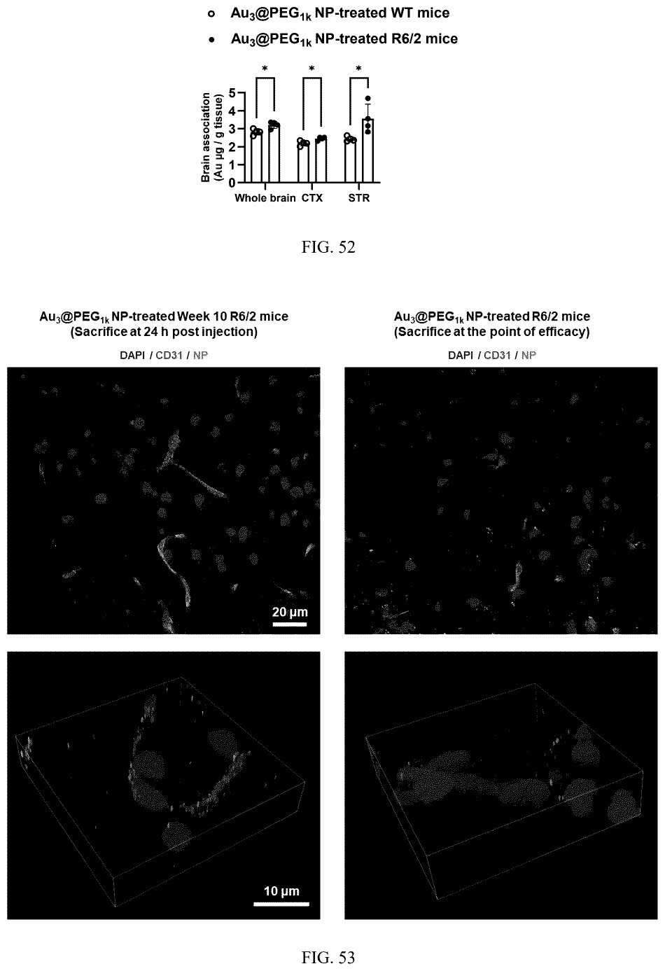

To assess the therapeutic efficacy of Au₃@PEG₁ₖ NPs, we i.v. injected the NPs weekly into R6/2 mice between the age of Week 6 (when HD began to develop) and Week 10 (FIG. 51). Mice were sacrificed at the age of Week 11, at the onset of late-stage HD symptoms [33]. ICP-MS measurements revealed higher gold contents in the whole brain, CTX, or STR of R6/2 mice than WT mice after 5 injections (FIG. 52). Confocal reflectance imaging showed less NP accumulation inside brain endothelial cells upon 5 injections than the 24 h post-injection time point (FIGS. 33 and 53), suggesting NP transit from the BBB to the brain parenchyma. Confocal immunofluorescence imaging verified NP accumulation mostly in microglial cells and, to a lesser extent, neurons and astrocytes in the cortex and striatum (FIGS. 54 and 34). As negative control, 70 kDa dextran NP, with a size similar to that of Au₃@PEG₁ₖ NP (Table 1), were i.v. injected at the same NP concentration weekly. As positive control, tetrabenazine [47] was intraperitoneally (i.p.) injected daily at the same cumulative weekly dose as literature precedent (2.5 mg/kg [48]). By counting 700 cells (neurons and microglia combined) in multiple brain sections, we found that 92% of neurons and 78% of microglia (FIG. 35), in the cortex and striatum combined, contained gold reflectance signals. Collectively, our data portrayed initial NP localization in the brain capillaries 24 h after a single injection, but such NP localization was no longer detectable after five weekly injections (FIG. 33); therefore, most NPs eventually exited the BBB and entered neurons in the adjacent brain tissue in cortex and striatum (FIGS. 36-41). Similarly, confocal images revealed the presence of gold NPs in neurons near the blood-CSF barrier after five weekly injections (e.g., vermal Purkinje cells around the fourth ventricle and hippocampal cortical neurons in the periventricular region (FIGS. 42-47), therefore we could not exclude transport to the brain via the blood-CSF barrier. Note that (i) we saline-perfused the brain to remove any free gold NP in the blood capillary lumen before ICP-MS measurements, (ii) the blood circulation half-life of gold NPs is 35 h (, which implies a limited level of gold NP in blood after five weekly injections, (iii) our confocal images of the HD brain in FIG. 33 depicted pronounced clearance of gold NPs from the BBB endothelium at the same timepoint, (iv) the injection route was i.v., not intranasal, intrathecal, or intracranial, (v) the background level of gold in the body is virtually nil. Thus, the gold contents in the brain after five weekly injections could only result from gold NP crossing the brain barriers and their release into the adjacent brain tissues.

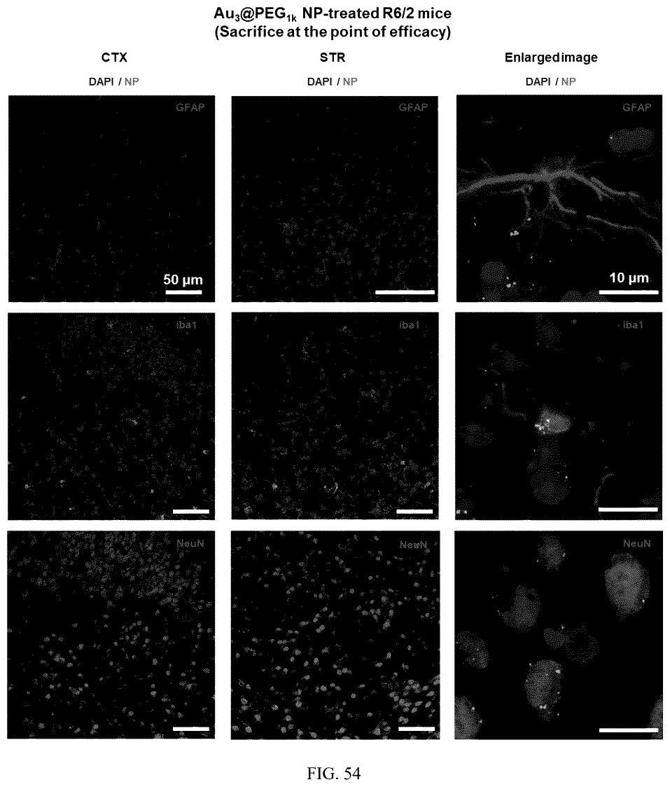

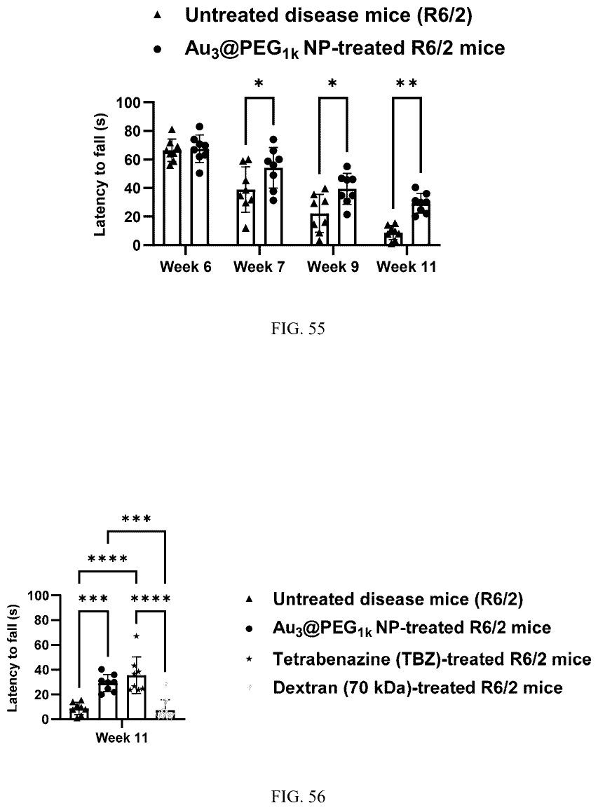

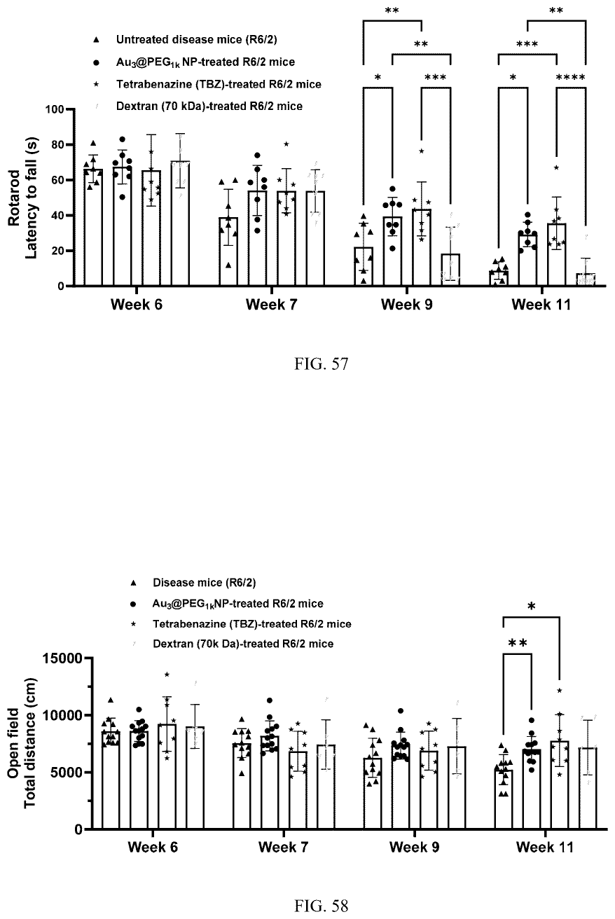

Utilizing the rotarod test (in which R6/2 mice are evaluated for their ability to stay on an accelerating rotating rod [49]), we observed delayed motor impairment in Au₃@PEG₁ₖ NP-treated mice relative to untreated mice as early as Week 7 (FIG. 55).

Initially, we used the rotarod test to evaluate the “latency to fall” of R6/2 mice, or the time duration for staying on an accelerating rotating rod [49]. As negative control, 70 kDa dextran NP, similarly sized as Au₃@PEG₁ₖ NP (), was i.v. injected at the same NP concentration weekly. As positive control, tetrabenazine was intraperitoneally (i.p.) injected daily at the same cumulative

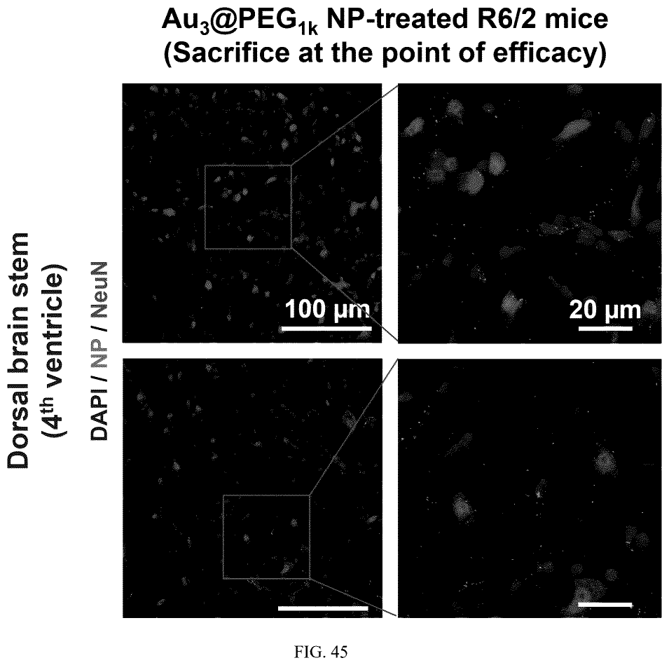

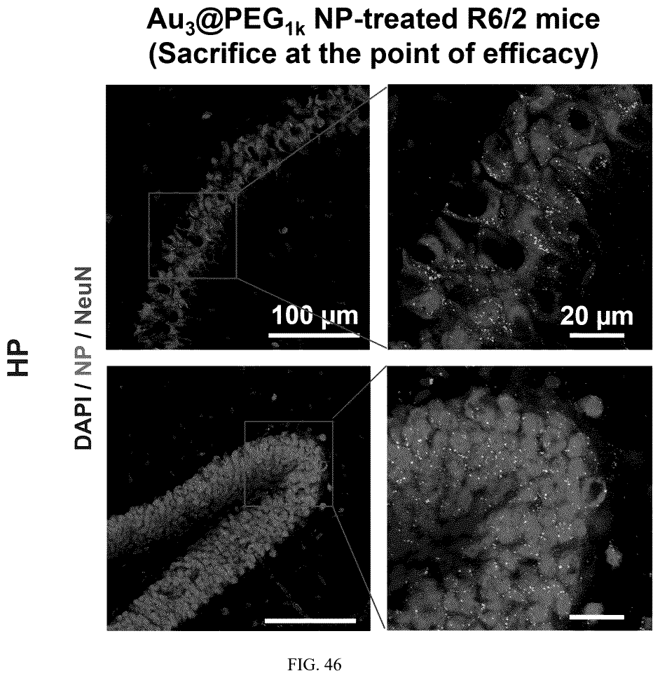

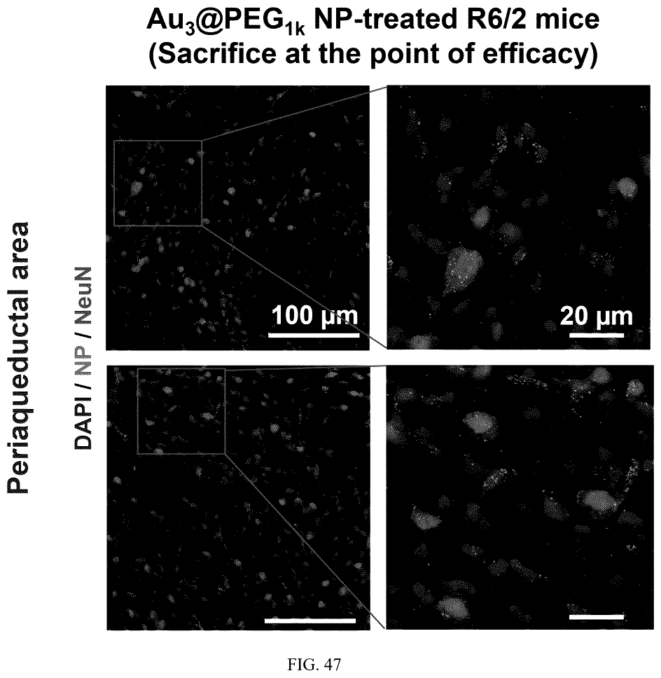

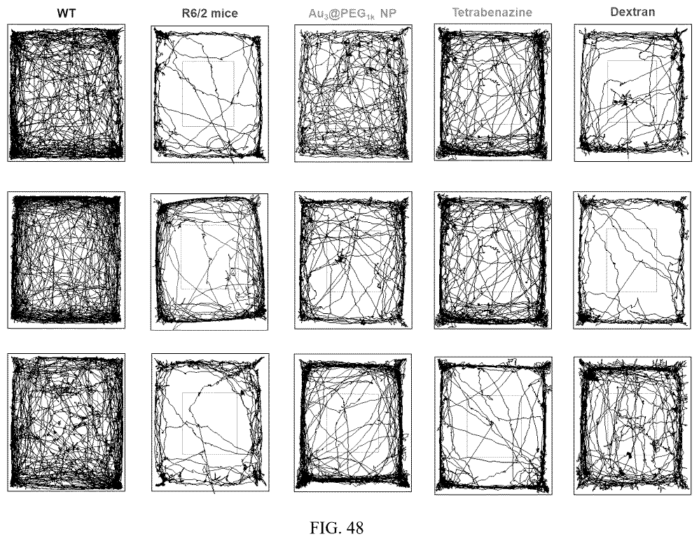

weekly dose as literature precedent [2.5 mg/kg; [48]]. Relative to their behavior at Week 6, the same group of mice at Week 7 showed 20% lower average latency to fall after receiving a single i.v. injection of Au₃@PEG₁ₖ NP or 40% lower average latency if left untreated. Therefore, one injection of NP ameliorated motor deficit by 50% (i.e., 20%÷40%) over a week (FIG. 28), justifying a weekly injection schedule. At Week 11, repeated injections of NPs ameliorated motor deficit by 67%, similar efficacy to tetrabenazine, but 70 kDa dextran NP did not show improvement (FIGS. 27, 28). Utilizing the open field test (in which R6/2 mice are tested on their ability to explore in an open field), we observed a ˜40% longer mean total walking distance in a given time duration (30 min) by R6/2 mice treated with Au₃@PEG₁ₖ NP relative to untreated mice, an efficacy result similar to that of tetrabenazine, but 70 kDa dextran NP showed no improvement (FIGS. 29 and 48).

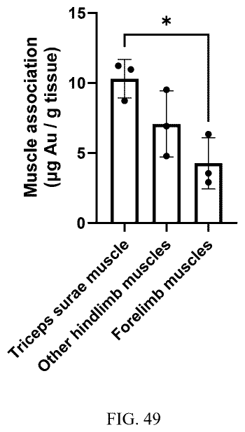

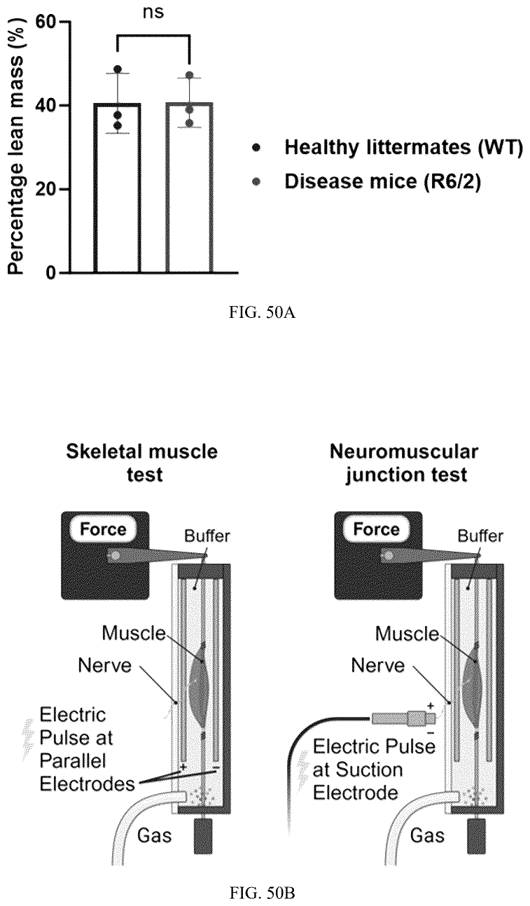

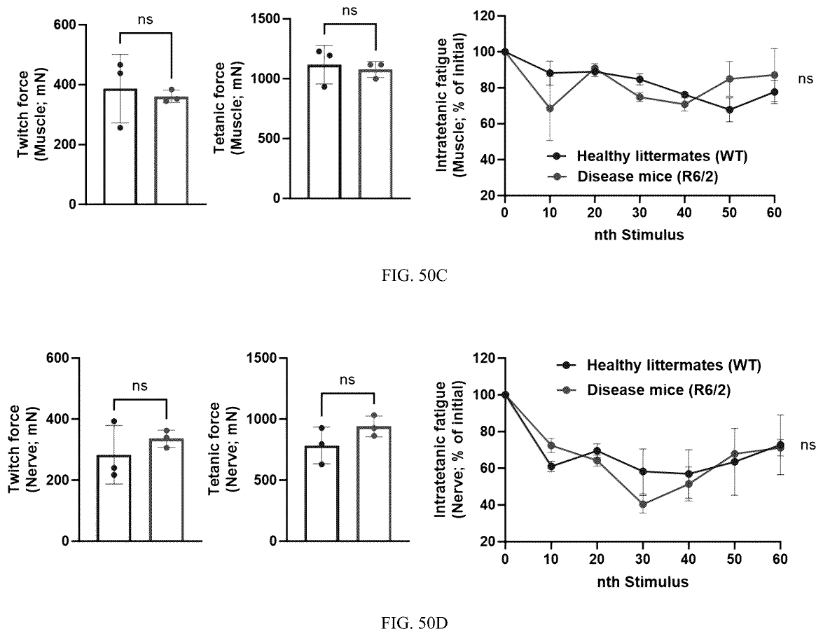

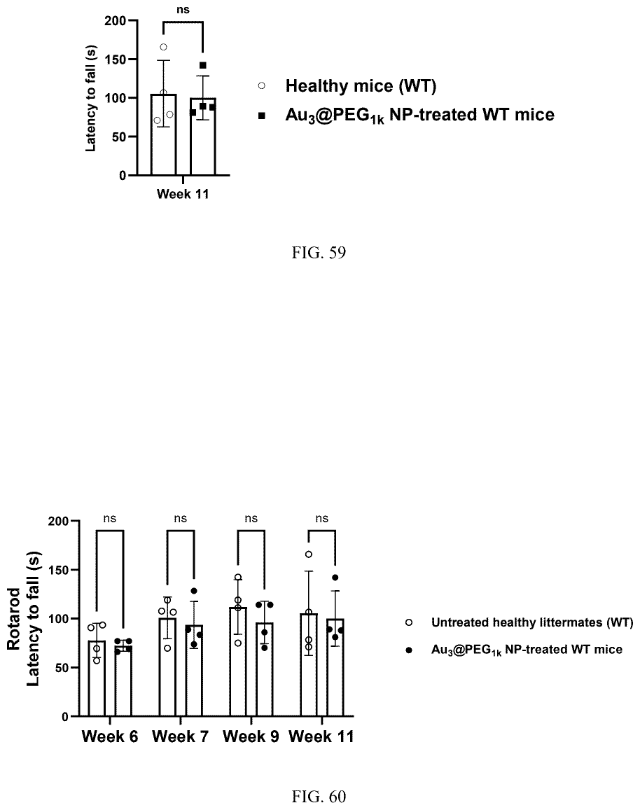

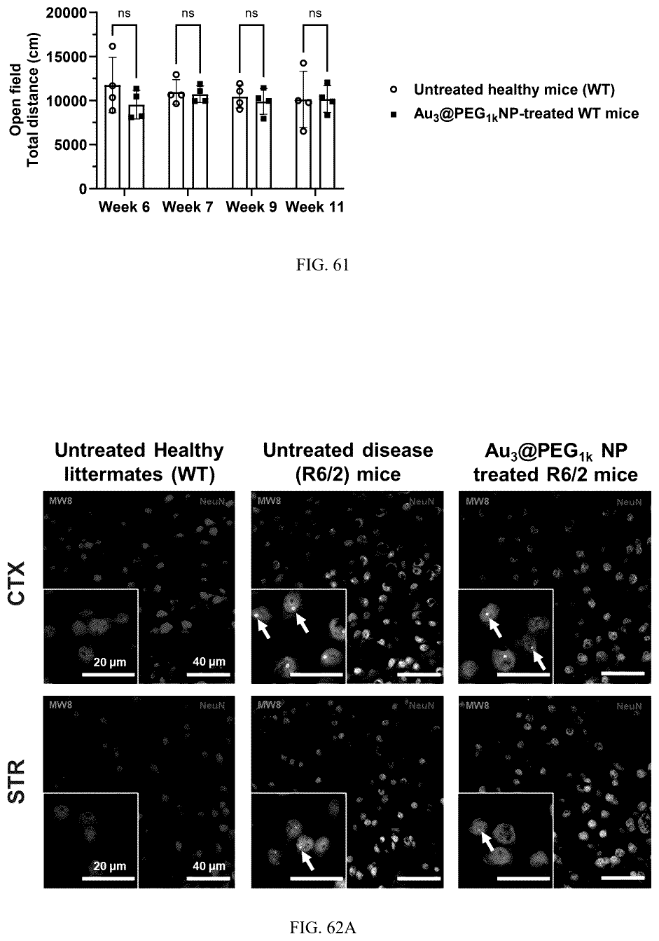

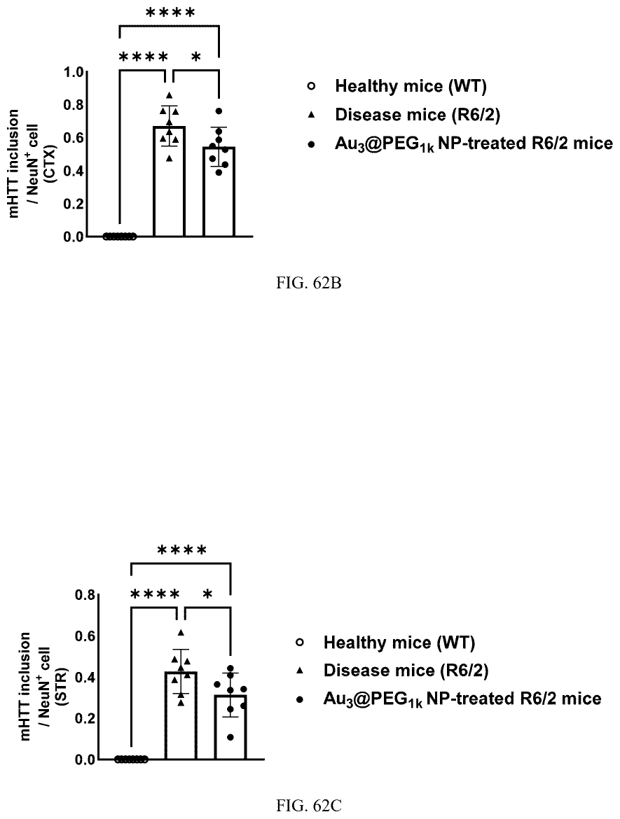

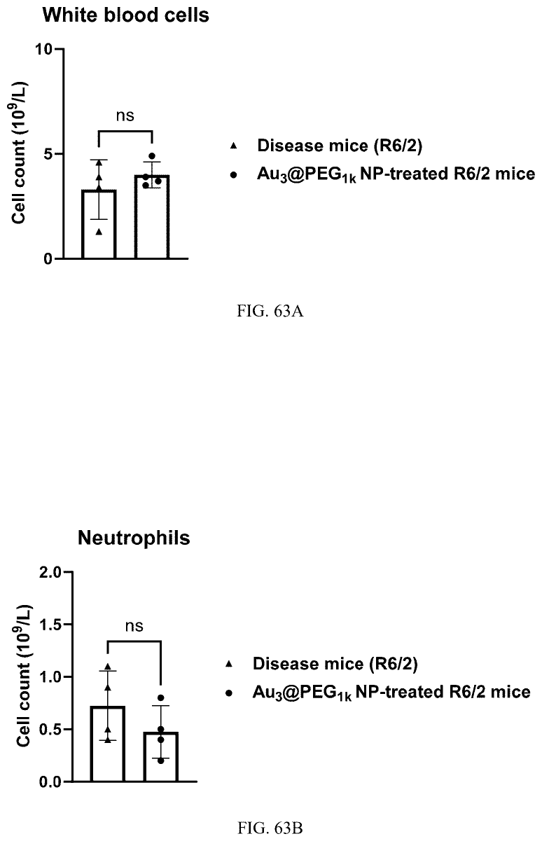

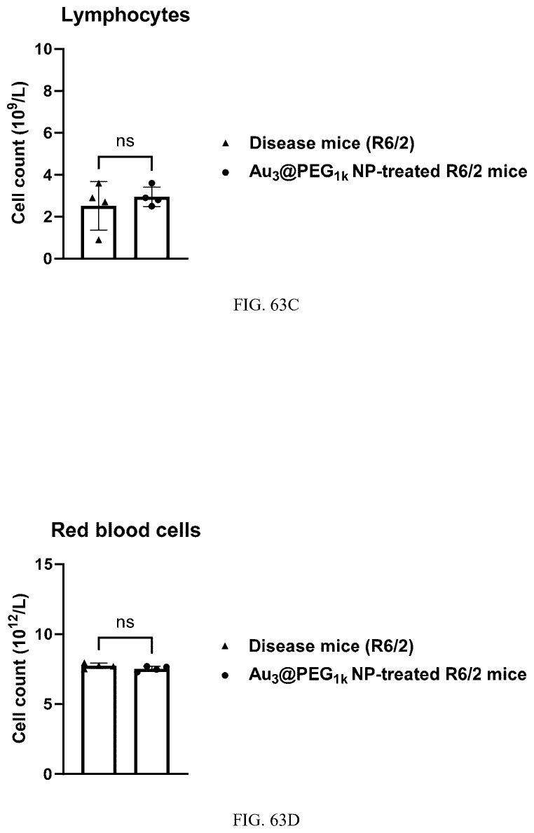

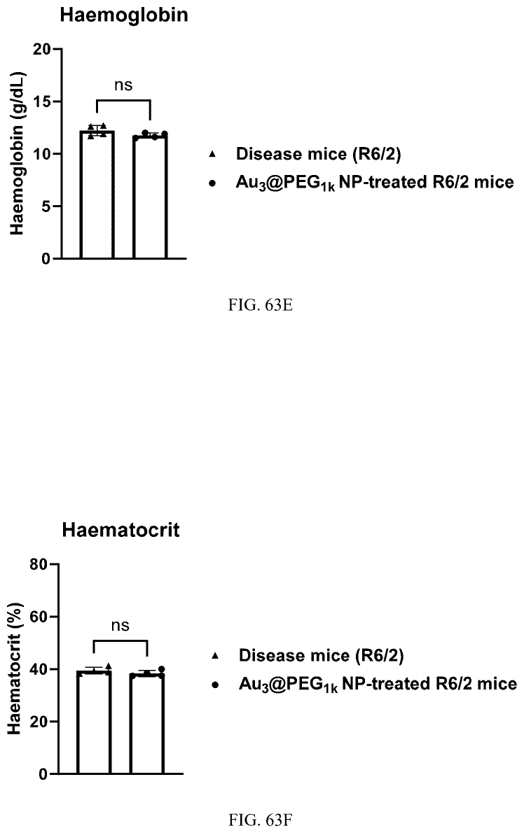

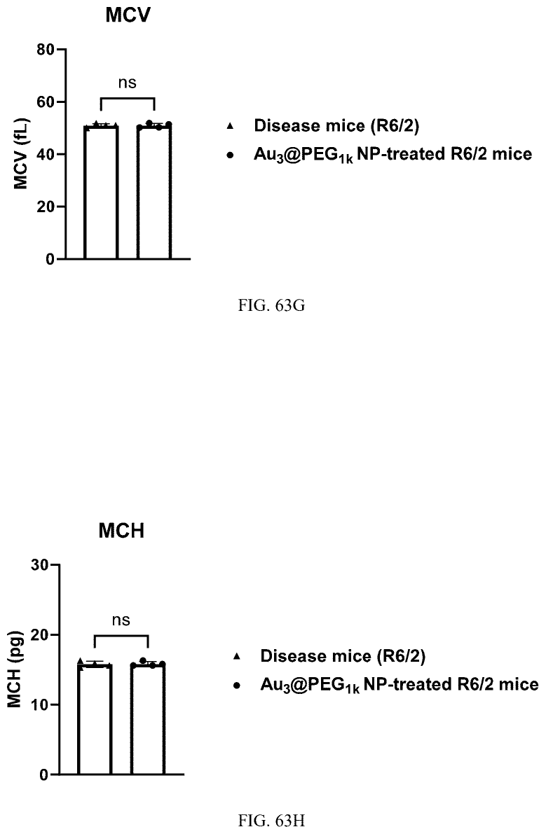

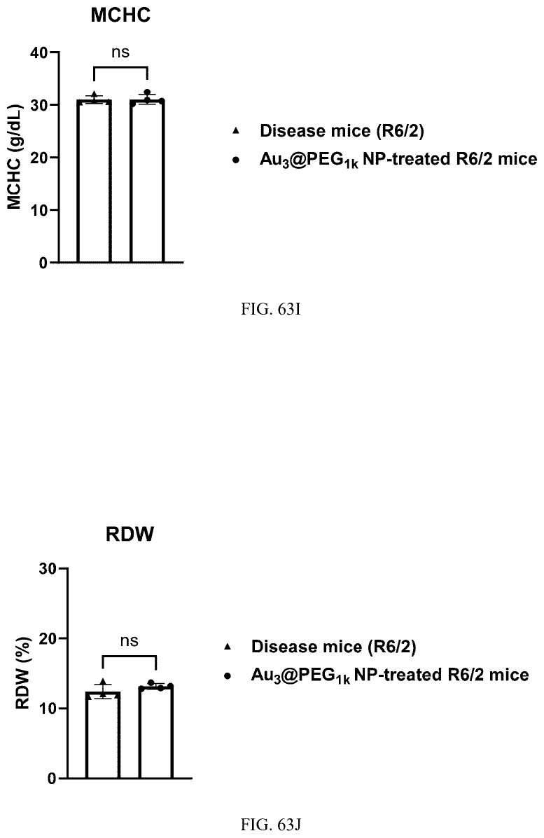

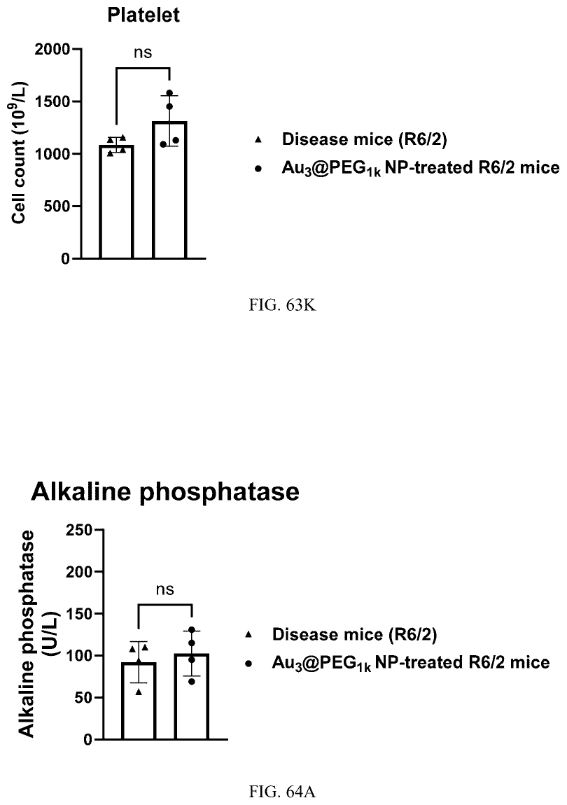

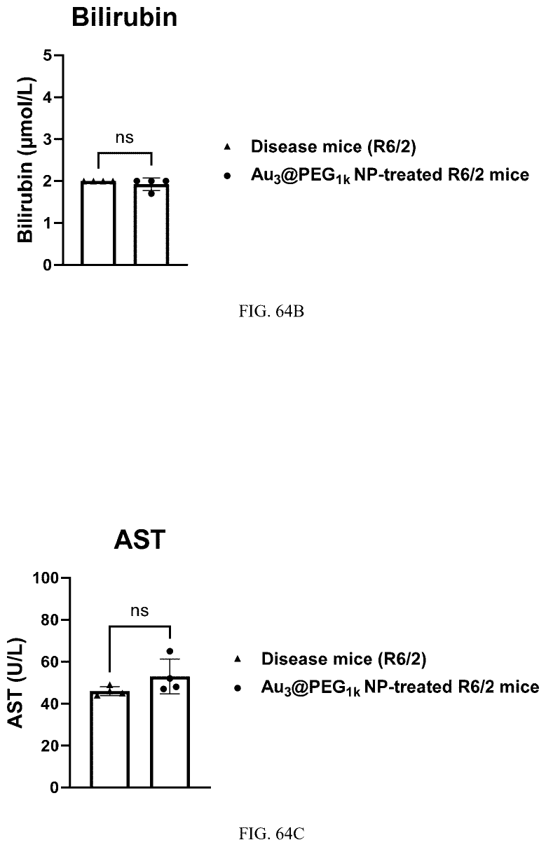

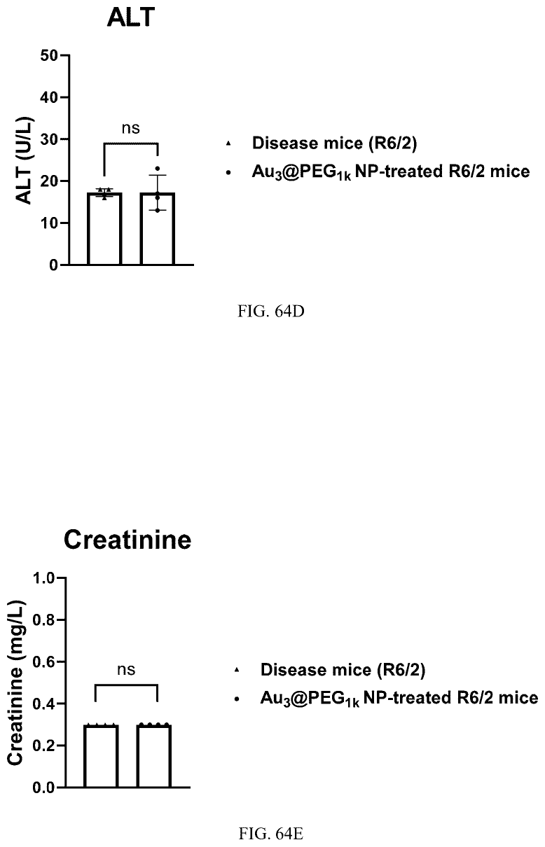

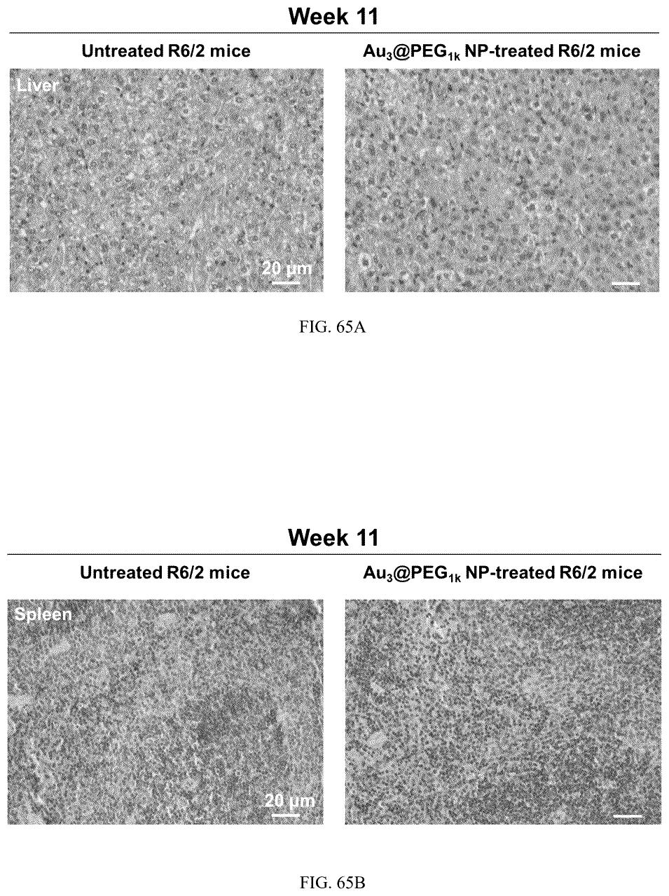

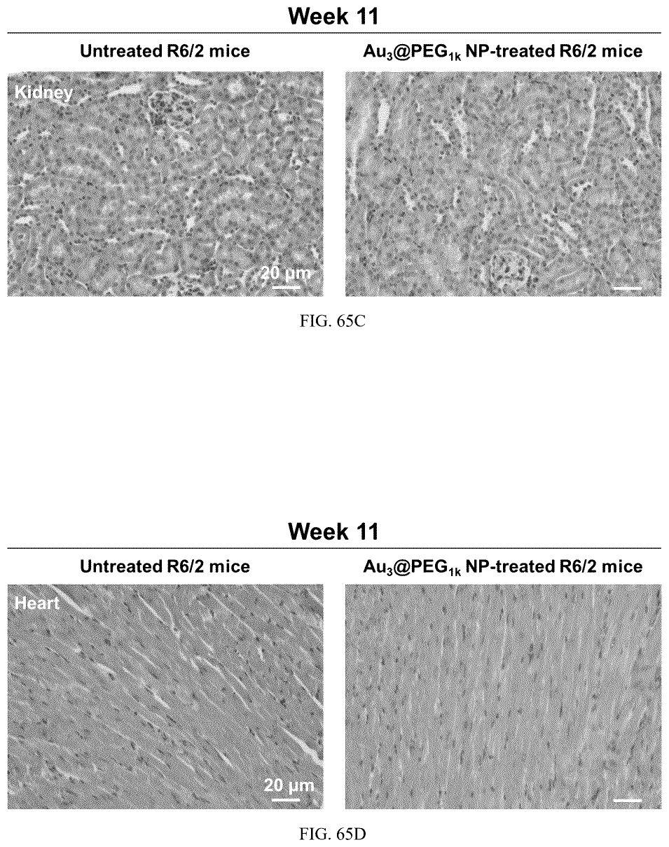

Our ICP-MS measurements revealed the presence of gold in the muscles of forelimbs and hindlimbs 24 h post-injection, most abundantly in the triceps surac muscles including the gastrocnemius and soleus (FIG. 49). Such NP distribution to peripheral skeletal muscles might suggest a therapeutic effect on improving motor function by locally acting on skeletal muscles or the neuromuscular junction rather than the HD brain, but this scenario is unlikely for two reasons. Firstly, we verified the limited degeneration of muscles and neuromuscular junctions in Week 11 R6/2 mice. Ex vivo functional tests revealed no difference in the twitch force, tetanic force, and intratetanic fatigue in the triceps surae muscle and the triceps surae-sciatic nerve complex (FIG. 50) between Week 11 R6/2 HD mice and age-matched healthy littermates. This result matches past reports on the emergence of muscle atrophy or neuromuscular abnormalities only for Week 12 R6/2 mice or older [50]. In both behavioral tests, we verified that Au₃@PEG₁ₖ NP did not alter the motor performance of healthy WT mice during the treatment period (FIGS. 59-61). The results indicated the anti-HD efficacy of Au₃@PEG₁ₖ NP. At the age of Week 11, confocal imaging revealed less mutant huntingtin aggregates in neurons of the cortex and striatum upon Au₃@PEG₁ₖ NP treatment (FIGS. 62A-62C). This observation suggested the therapeutic potential of Au₃@PEG₁ₖ NP for modifying HD at the protein level and matched past reports on using gold NPs for inhibiting Alzheimer's amyloid-β fibrillization [51] and Parkinson's α-synuclein aggregation [29]. There is no change in hematology (FIGS. 63A-63K), functions of the liver and kidney (FIGS. 64A-64E), or tissue morphology in major organs (FIG. 67), suggesting limited short-term systemic toxicity.

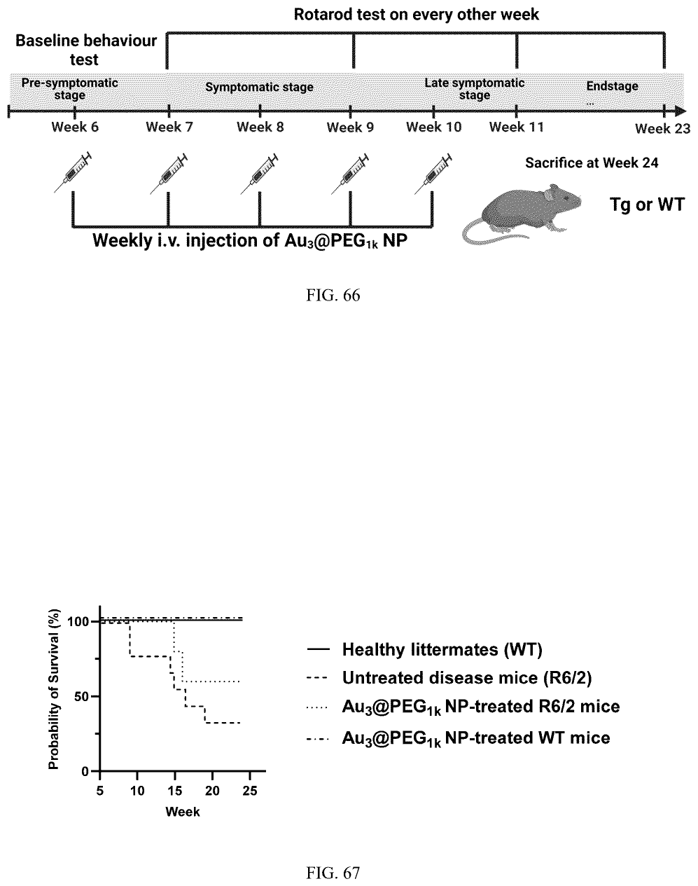

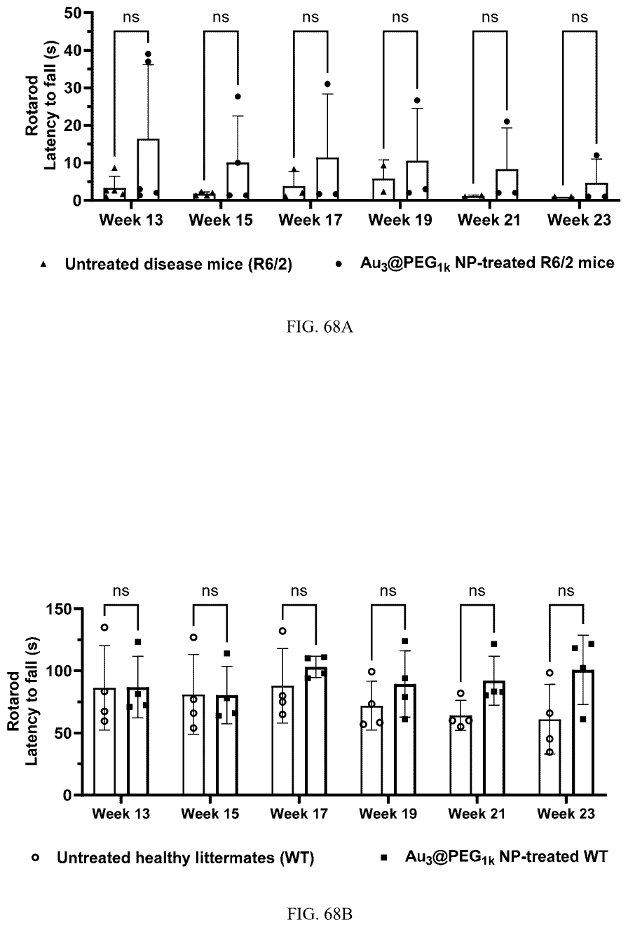

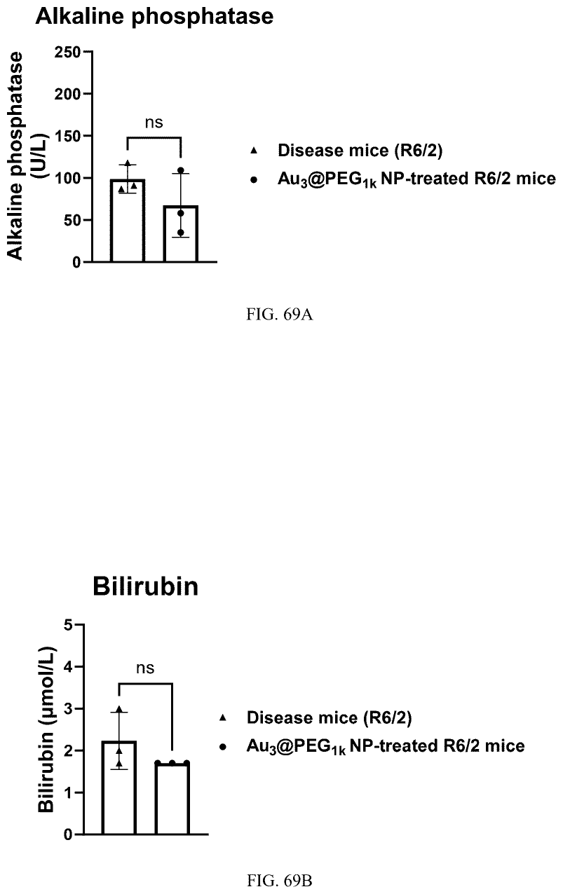

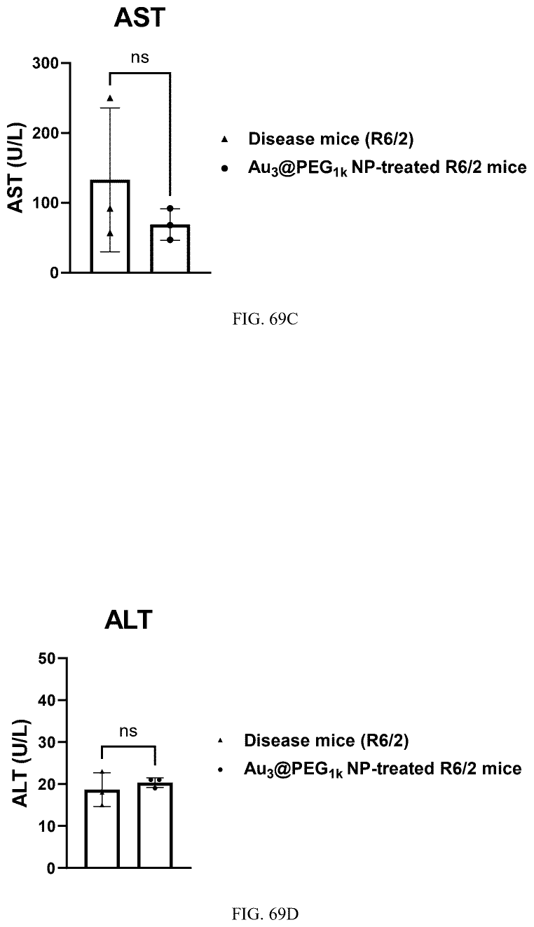

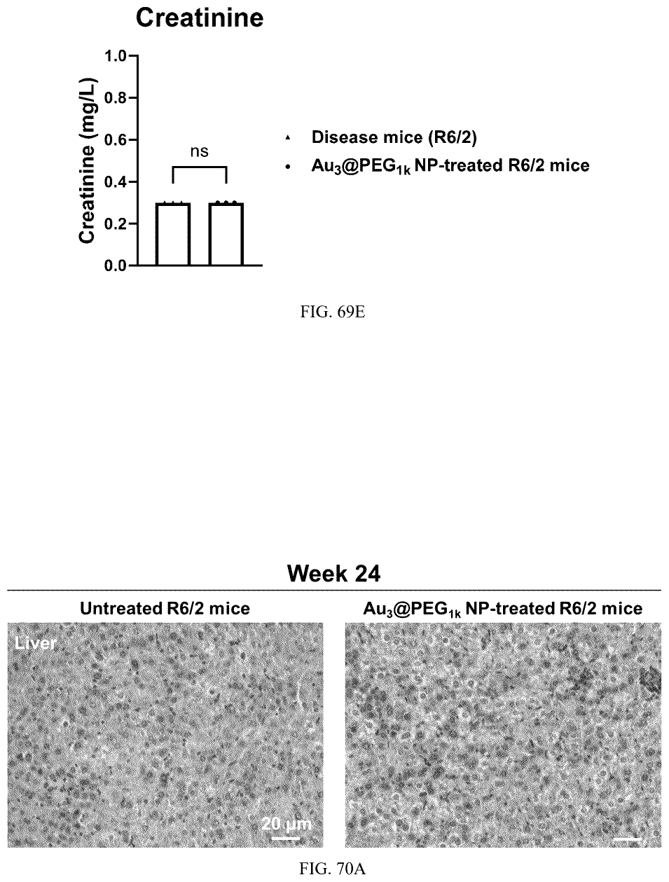

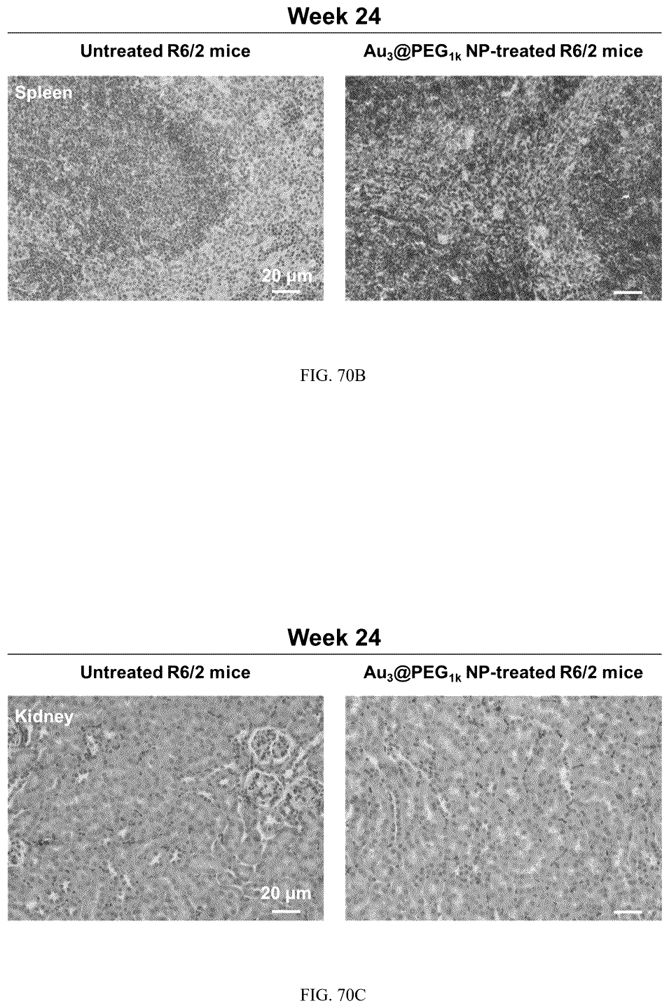

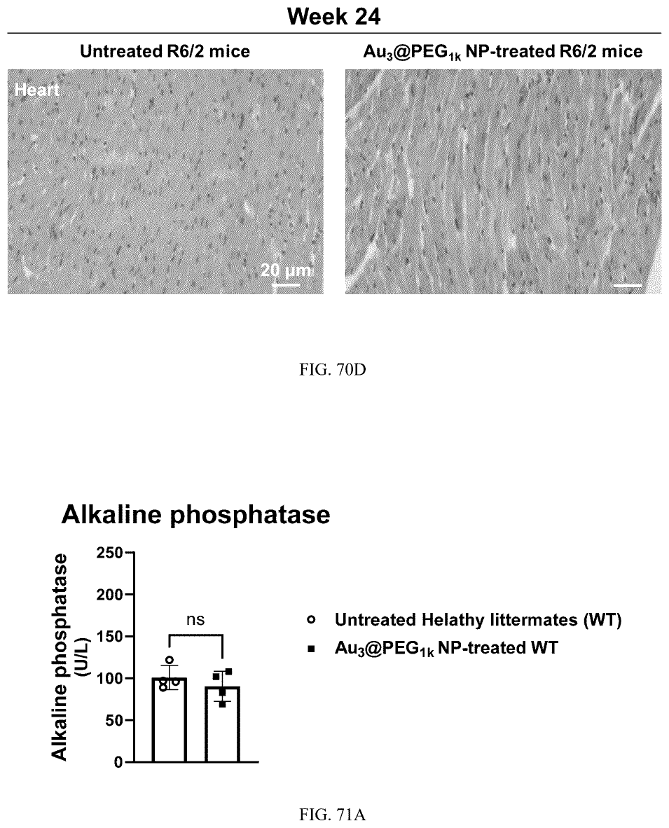

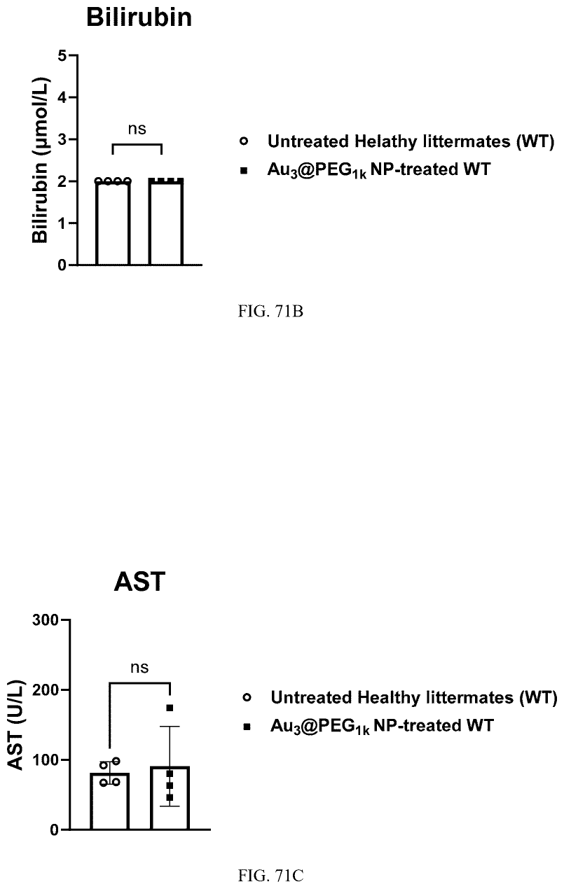

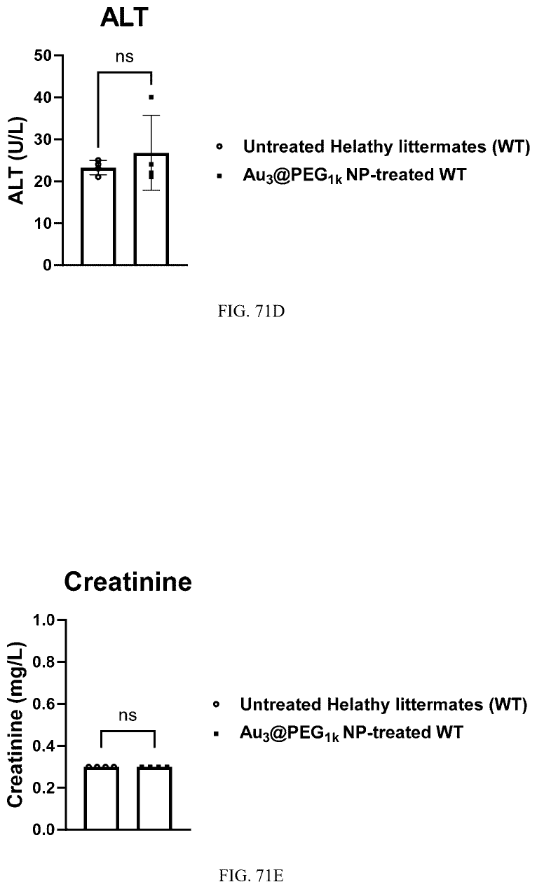

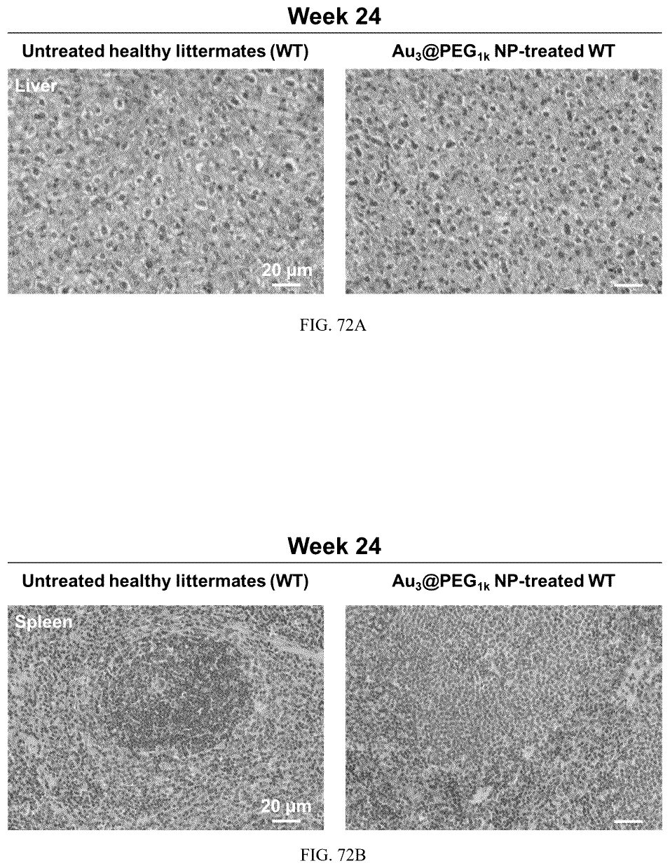

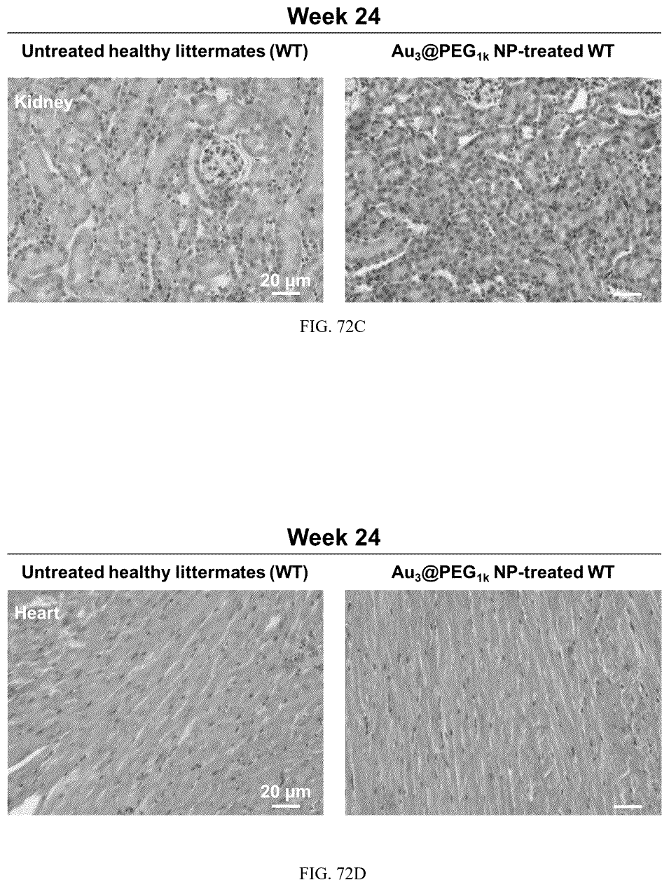

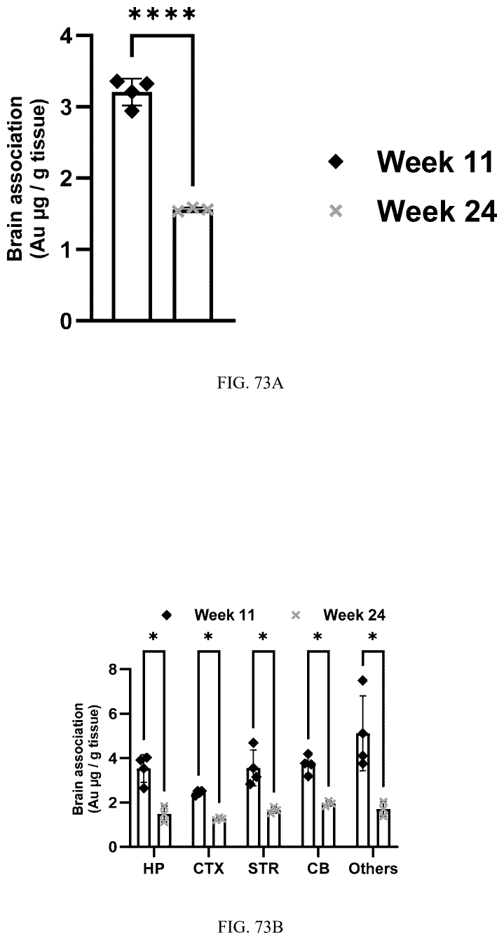

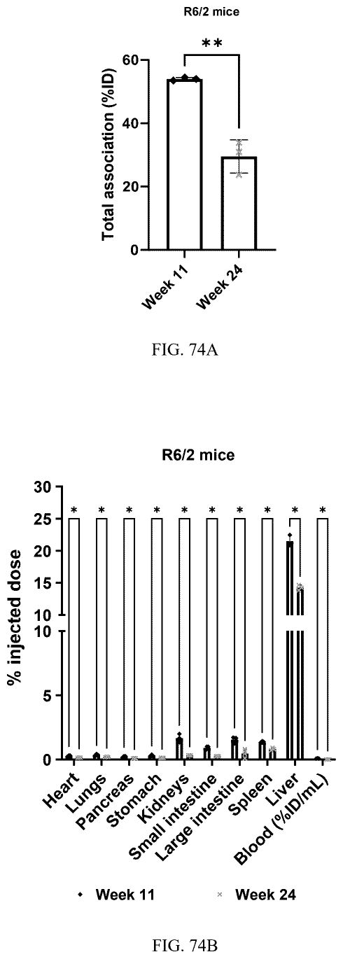

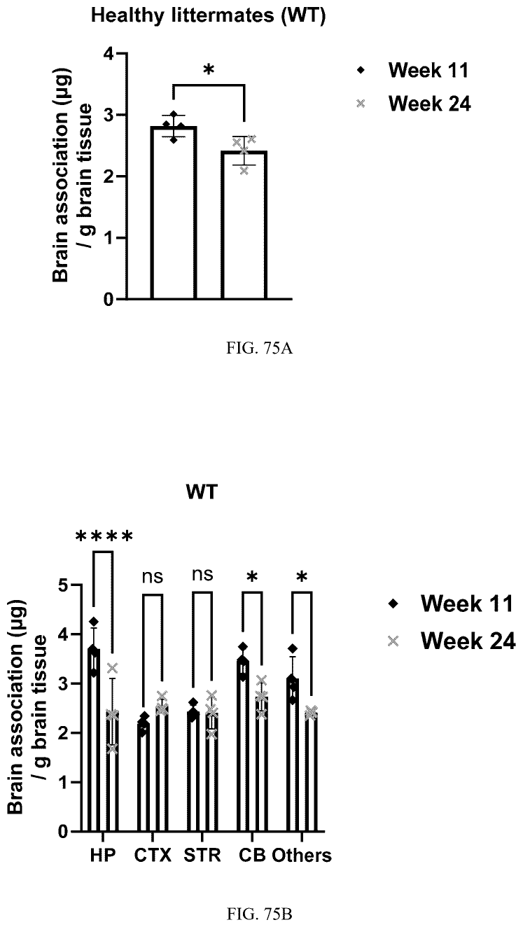

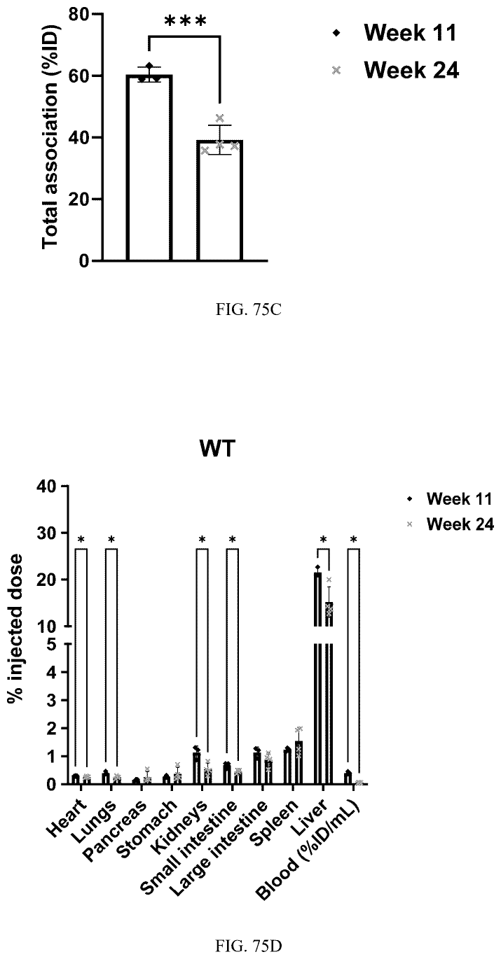

The long-term efficacy and toxicity of Au₃@PEG₁ₖ NP was up to 14 weeks post-treatment (at the age of Week 24 when R6/2 mice develop late-stage HD; FIG. 66). Notably, Au₃@PEG₁ₖ NPs prolonged the survival of R6/2 mice relative to untreated R6/2 mice (FIG. 67), without affecting the survival or motor behavior of healthy WT mice (FIGS. 67 and 68A-68B). At the point of sacrifice on Week 24, there was again no change in liver and kidney functions as well as tissue morphology of major internal organs in both R6/2 (FIGS. 69A-69E and 70A-70D) and WT (FIGS. 71A-71E and 72A-72D) mice. Furthermore, ICP-MS measurements showed pronounced reduction in gold contents in the whole brain, various tissue compartments of the brain (FIGS. 72A and 73A-73B), and other major organs relative to Week 11 in R6/2 mice (FIGS. 74A-74B). For healthy WT mice, the present invention detected similar reduction in gold contents in the whole brain and other major internal organs from Weeks 11 to 24 (FIGS. 75A-75D). In summary, Au₃@PEG₁ₖ NP exhibited anti-HD efficacy without inducing toxicity in the long term.

### EXAMPLE 3

### Upregulation of Oxidative Phosphorylation

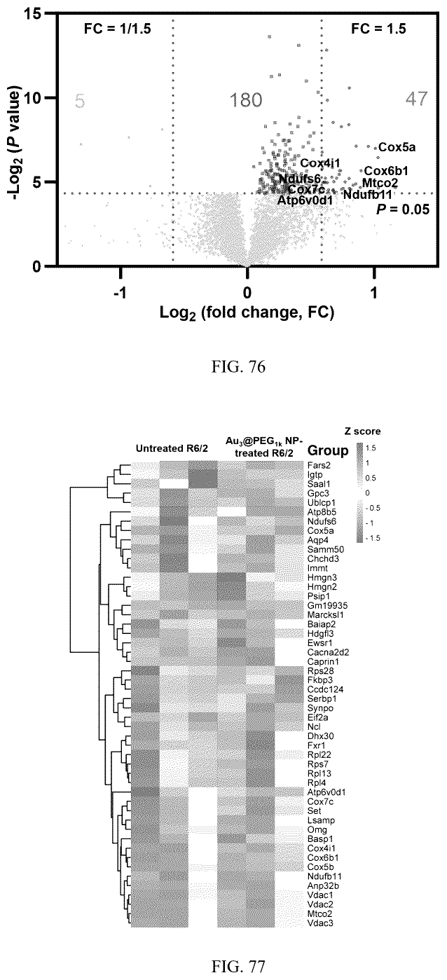

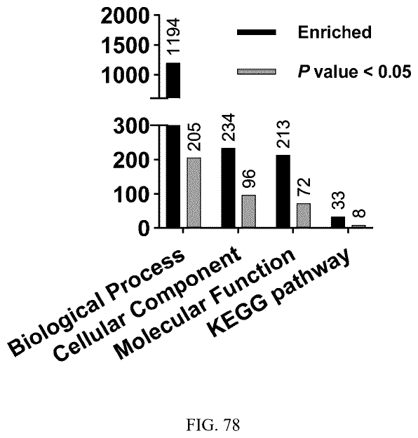

To elucidate the anti-HD mechanism of Au₃@PEG₁ₖ NP, we initially utilized unbiased proteomics analysis to identify changes in protein expression in the HD brain of R6/2 mice on Week 11, based on the treatment regimen in FIG. 51. We detected 52 differentially expressed proteins (DEPs) in the pairwise comparison between the Au₃@PEG₁ₖ NP and untreated groups (Au₃@PEG₁ₖ NP vs. untreated), with a cut-off fold change (FC) ≥1.5 or FC ≤1/1.5 and a P value <0.05 (FIGS. 76 and 77). Of the 8 enriched Kyoto Encyclopedia of Genes and Genomes (KEGG) pathways identified, “oxidate phosphorylation” was the most enriched (Table 4 and FIG. 78).

TABLE 4
Enriched HD-related Kyoto Encyclopedia of Genes and
Genomes (KEGG) pathways from the same comparison, with
DEPs related to oxidative phosphorylation bolded.

| KEGG pathway | P value | Genes |
|---|---|---|
| Oxidative | 8.1 × 10⁻⁹ | Cox5a, Cox5b, Cox6b1, Mtco2, Ndufb11, |
| Phosphorylation | | Cox4i1, Cox7c, Atp6v0d1, Ndufs6 |
| Parkinson | 2.7 × 10⁻⁷ | Cox5a, Cox5b, Cox6b1, Mtco2, Ndufb11, |
| disease | | Cox4i1, Vdac1, Cox7c, Ndufs6 |
| Huntington | 1.1 × 10⁻⁶ | Cox5a, Cox5b, Cox6b1, Mtco2, Ndufb11, |
| disease | | Cox4i1, Vdac1, Cox7c, Ndufs6 |
| Alzheimer | 5.7 × 10⁻⁵ | Cox5a, Cox5b, Cox6b1, Mtco2, Ndufb11, |
| disease | | Cox4il, Cox7c, Ndufs6 |

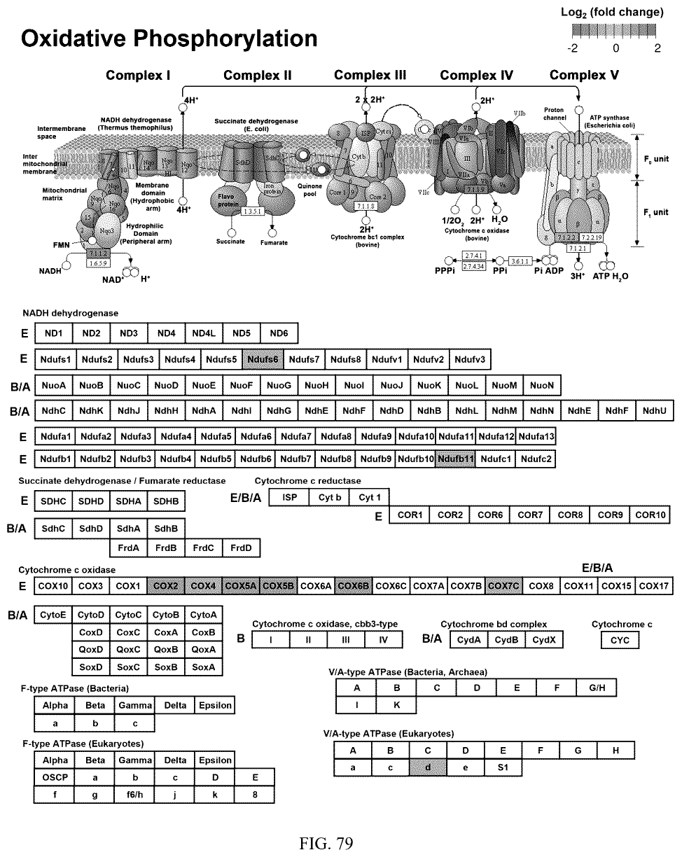

The “HD” KEGG pathway was also enriched, with 8 out of 9 of its constituent DEPs related to oxidative phosphorylation (Table 4 and FIG. 79) and all were found upregulated (FIG. 77). Such regulated DEPs are cytochrome c oxidase (Cox4i1, Cox5a, Cox5b, Cox6b1, Cox7c, Mtco2), nicotinamide adenine dinucleotide hydride (NADH) dehydrogenase (Ndufb11, Ndufs6), and V-ATPase (ATP6VOD1). In oxidative phosphorylation, the electrons derived from NADH (which regulates cellular energy metabolism in glycolysis [52]) break down oxygen to release energy and fuel the synthesis of adenosine triphosphate (ATP) [53]. Recent in vitro evidence showed that gold NPs catalyze the conversion of NADH to its oxidized counterpart (NAD⁺) for activating ATP production in cells [54], echoing our in vivo result that Au₃@PEG₁ₖ NP upregulate oxidative phosphorylation. Incidentally, the KEGG pathways of “Parkinson's disease” and “Alzheimer's disease” were also enriched because they share the similarly upregulated DEPs as those in the “HD” KEGG pathway (Table 4), a result consistent with past reports on the efficacy of gold NPs against both neurodegenerative disorders.

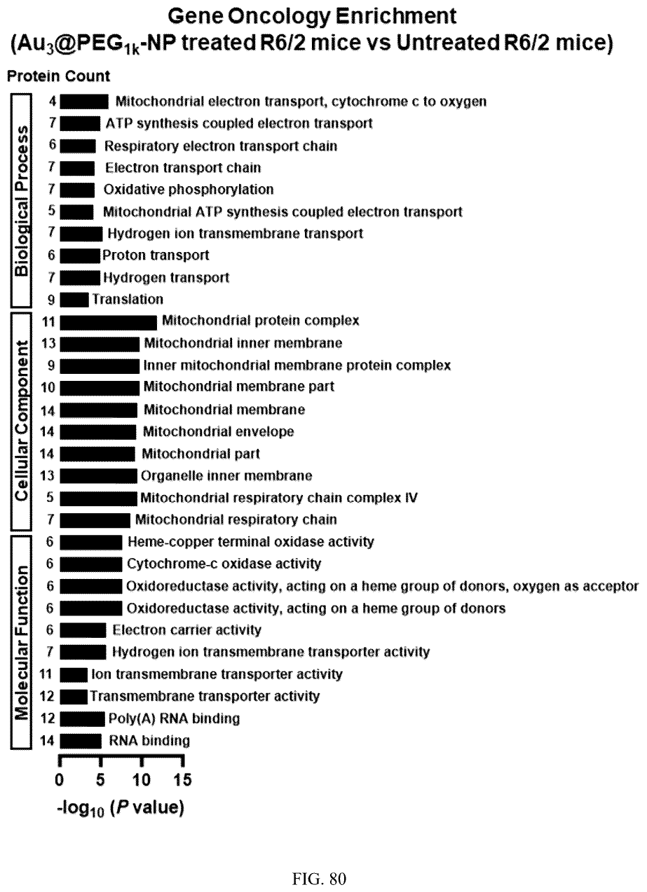

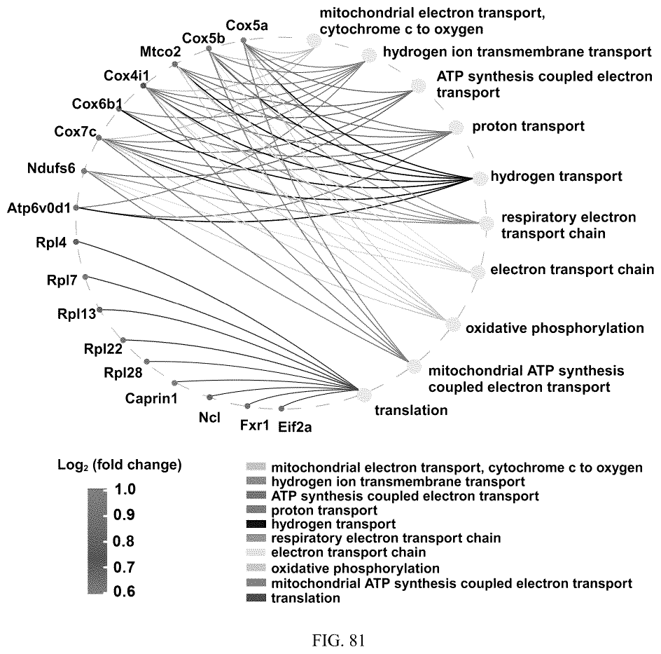

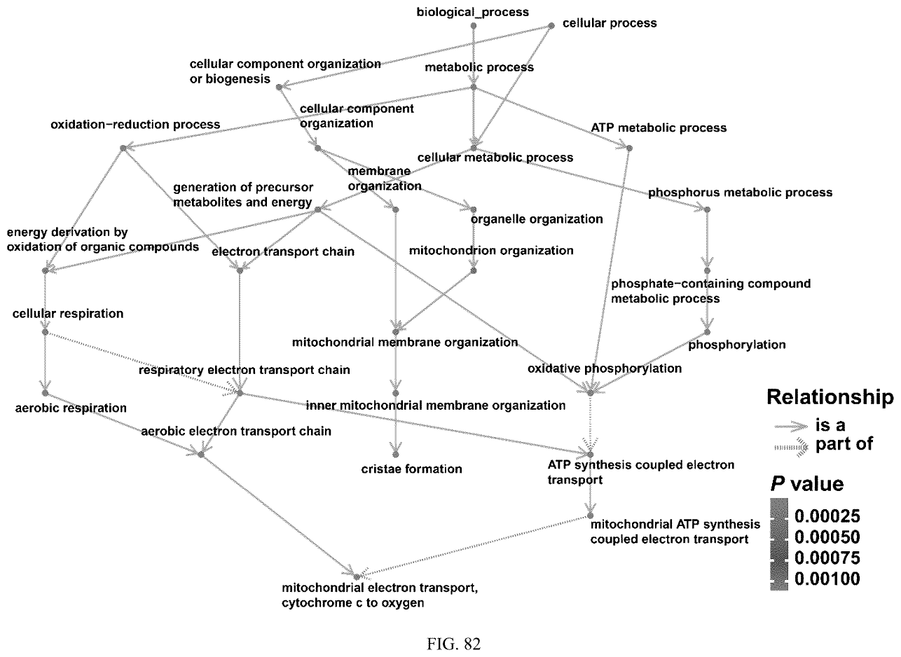

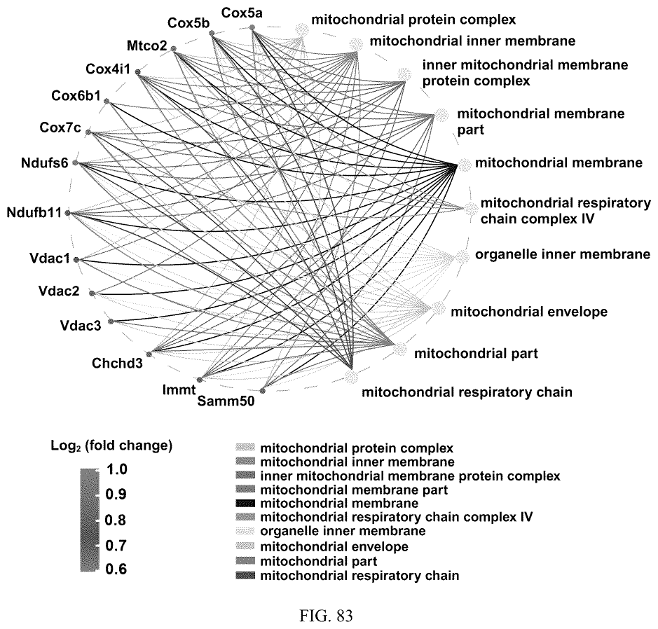

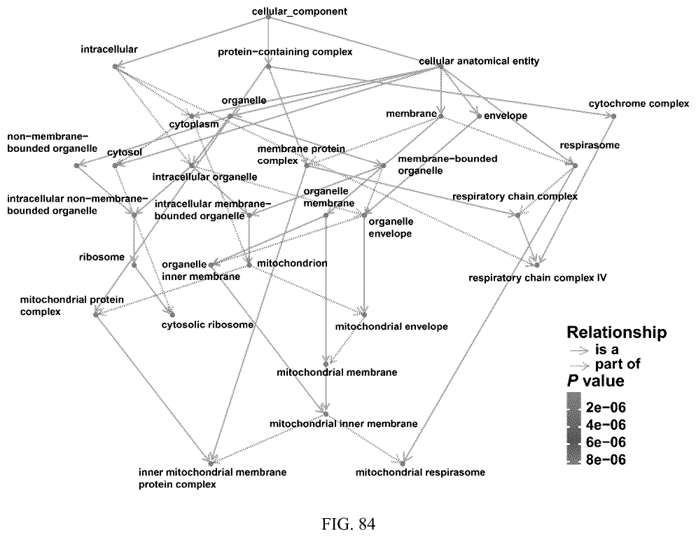

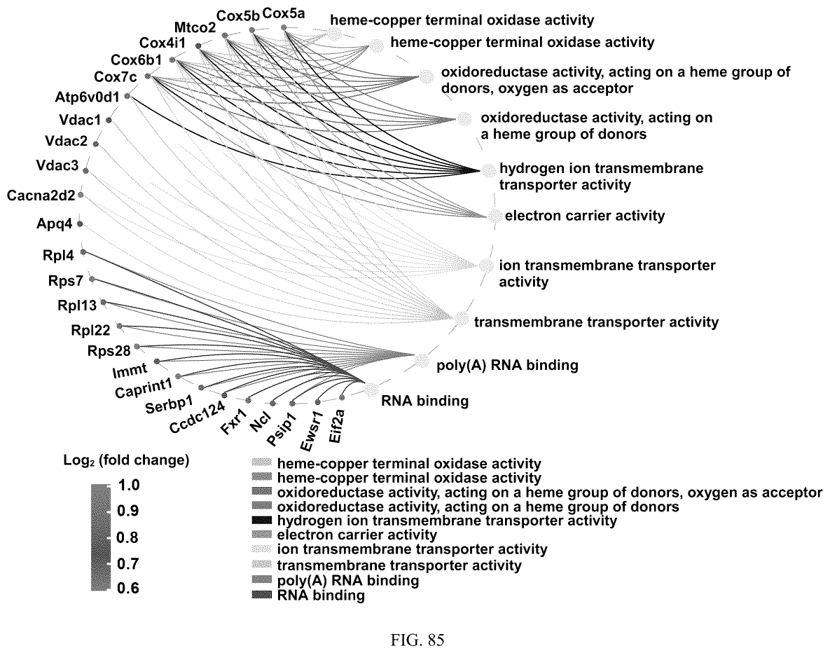

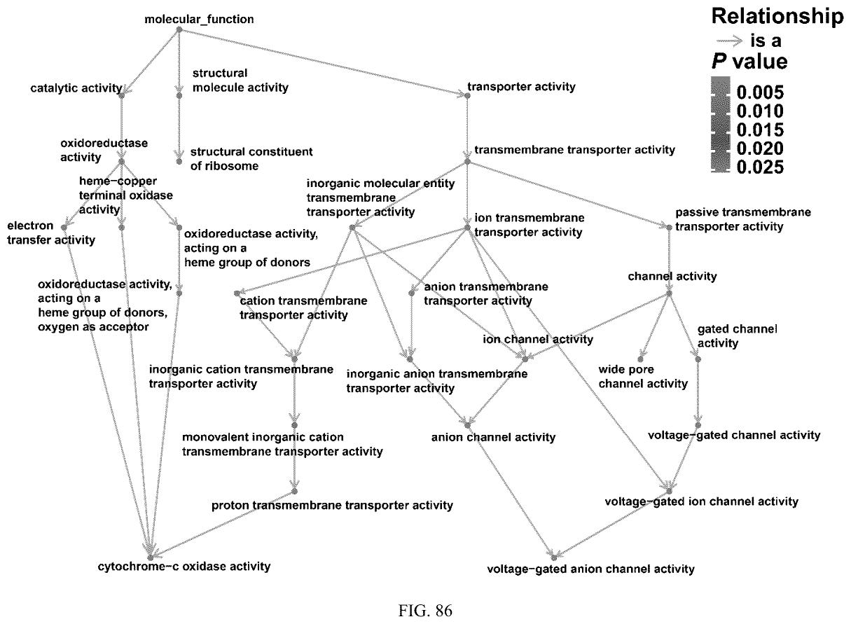

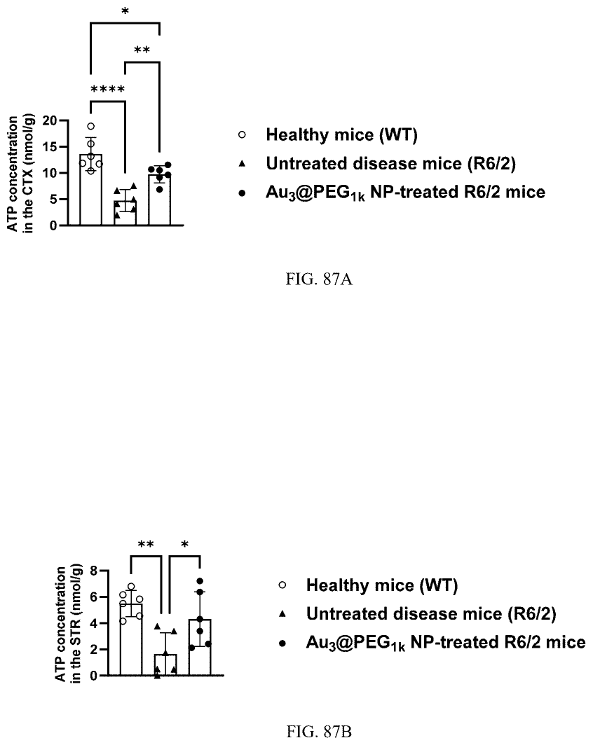

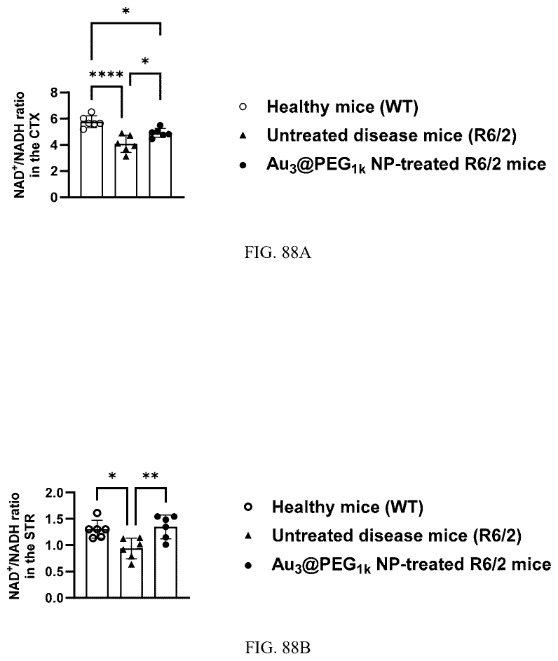

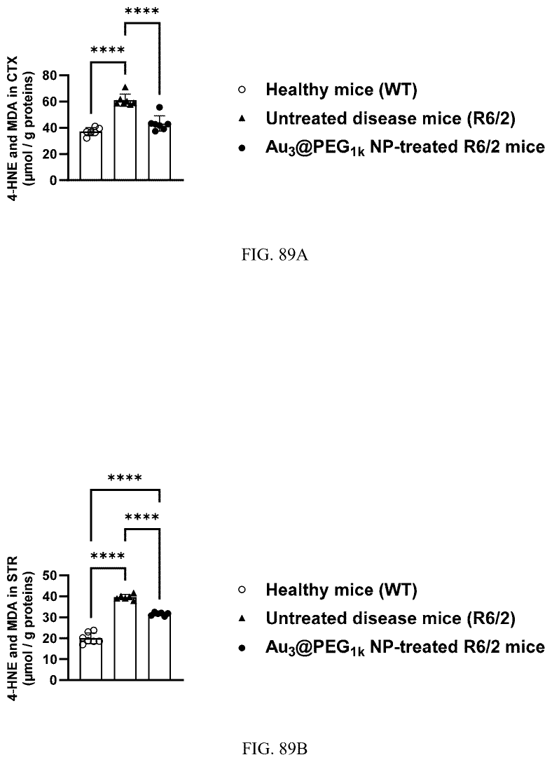

23 out of the 52 DEPs were upregulated and related to the top ten significantly enriched gene ontology (GO) terms (FIGS. 77 and 80). On biological process, most GO terms are clinically relevant to HD [55], such as “ATP (adenosine triphosphate) synthesis coupled electron transport” and “respiratory electron transport chain” (FIGS. 80-82). On cellular component, the GO terms are related to respirasome and mitochondrion (FIGS. 83 and 84). On molecular function, most GO terms are related to “oxidoreductase activity” and “transmembrane transporter activity” (FIGS. 85 and 86). As oxidative phosphorylation was downregulated in HD patients and mice [56, 57], these data prompted us to hypothesize that the therapeutic action of Au₃@PEG₁ₖ NP stems from its upregulation of oxidative phosphorylation and cellular activities related to ATP metabolism in the HD brain. To test this hypothesis, the present invention shows that Au₃@PEG₁ₖ NP treatment enhanced the level of ATP (FIGS. 87A and 87B) and the ratio of NAD⁺ to NADH (FIGS. 88A and 88B) in the striatum and cortex of R6/2 mice, consistent with the biological process of “generation of precursor metabolites and energy”. Lastly, the present invention considered the involvement of lipid peroxidation, whereby oxidants (e.g., free radicals) attack lipids containing C-C double bond (e.g., polyunsaturated fatty acids [58]). The data showed that Au₃@PEG₁ₖ NP treatment attenuated the levels of 4-hydroxynonenal (HNE) and malondialdehyde (MDA), both markers of lipid peroxidation, in the cortex and striatum (FIGS. 89A and 89B). Such attenuated oxidative stress suggested improved mitochondrial function [59], a result consistent with the biological process of “mitochondrion organization”.

### EXAMPLE 4

### Inhibition of p38α Phosphorylation and PDK1

We conducted kinome profiling to unbiasedly identify potential pharmacological targets of Au₃@PEG₁ₖ NP that may account for the upregulation of oxidative phosphorylation. Of the 281 kinases screened, ˜200 nM Au₃@PEG₁ₖ NPs inhibited 27 kinases by >85%, with 8 of them related to HD based on literature precedent. Furthermore, of these 8 HD-related, NP-inhibited kinases, the two most studied are MAPK14 (p38α) and PDK1 (Table 5).

TABLE 5
List of kinase with >85% inhibition in their activities after treatment
with ~200 nM of Au₃@PEG₁ₖ NP (with corresponding kinase comparing
against with ~200 nM of 70 kDa Dextran) using the Z′-LYTE ™ Kinase
Assay Kit (Thermo Fisher Scientific).

| | | Mean Percent Inhibition (%) | | HD-related |
|---|---|---|---|---|
| | [ATP] tested | | 70 kDa | publication |
| Kinase | (μM) | Au₃@PEG₁ₖ NP | Dextran NP | count^{a} |
| MAPK14 (p38α) | Cascade: 100 | 100 | 50 | 53 |
| PDK1 Direct | Km app: 27 | 85 | 1 | 6 |
| JAK2 | Km app: 31 | 91 | 11 | 4 |
| MAP2K6 (MKK6) | Cascade: 100 | 100 | 3 | 2 |
| PLK2 | Km app: 29.6 | 99 | 10 | 2 |
| CAMK4 (CaMKIV) | Km app: 18 | 91 | 9 | 2 |
| GRK4 | Km app: 12 | 92 | 64 | 1 |
| NEK1 | Km app: 118.7 | 86 | 9 | 1 |
| FGR | Km app: 10 | 90 | 47 | 0 |
| PLK3 | Km app: 47.8 | 87 | 50 | 0 |
| MELK | Km app: 30 | 100 | 53 | 0 |
| RPS6KA6 (RSK4)_1hr | Km app: 30 | 99 | 31 | 0 |
| BRSK1 (SAD1) | Km app: 32 | 97 | 15 | 0 |
| RPS6KA4 (MSK2) | Km app: 14.1 | 92 | 20 | 0 |
| SRMS (Srm) | Km app: 126.9 | 89 | 15 | 0 |
| RPS6KB2 (p70S6Kb) | Km app: 44 | 88 | 0 | 0 |
| MAP4K5 (KHS1) | Km app: 55 | 85 | 39 | 0 |

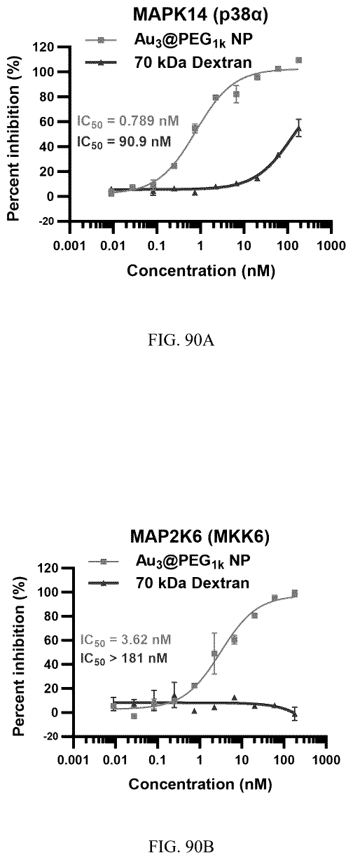

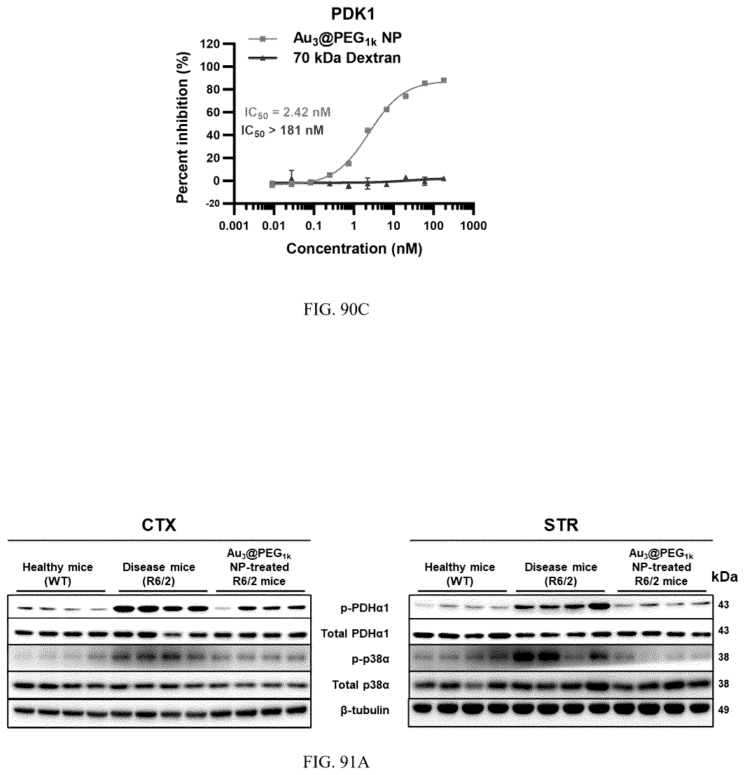

The half-maximal inhibitory concentrations (IC₅₀'s) of Au₃@PEG₁ₖ NP for p38α,MAP2K6 (MKK6; an upstream kinase of p38), and PDK1 were 0.789, 3.62, and 2.42 nM respectively. These IC₅₀ values, pronouncedly lower than those of the negative control 70 kDa dextran NP, underscore the high potency of Au₃@PEG₁ₖ NP (FIGS. 90A-90C). p38α is involved in neuroinflammation [60], and p38 phosphorylation (p-p38) was found increased in the striatum of HD patients and mouse models, an event associated with neuronal death and excitotoxicity [62]. A previous clinical trial (NCT03980938) centered on the use of neflamapimod, a p38α inhibitor, to treat HD. Activation of PDK1, caused by upregulation of Wnt/β-catenin signaling [63, 64], blocked pyruvate dehydrogenase (PDH) and conversion of pyruvate into acetyl-coenzyme A in the Kreb cycle, giving rise to anaerobic glycolysis and inefficient ATP generation [65]. Dichloroacetate, a PDK1 inhibitor now under clinical tests for treating glioblastoma (NCT05120284), could also treat HD in mouse models by redirecting the cells to aerobic respiration (i.e., oxidative phosphorylation).

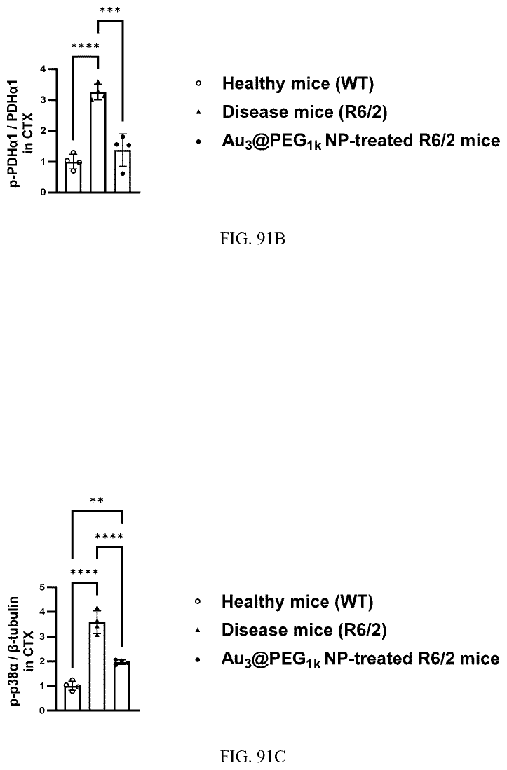

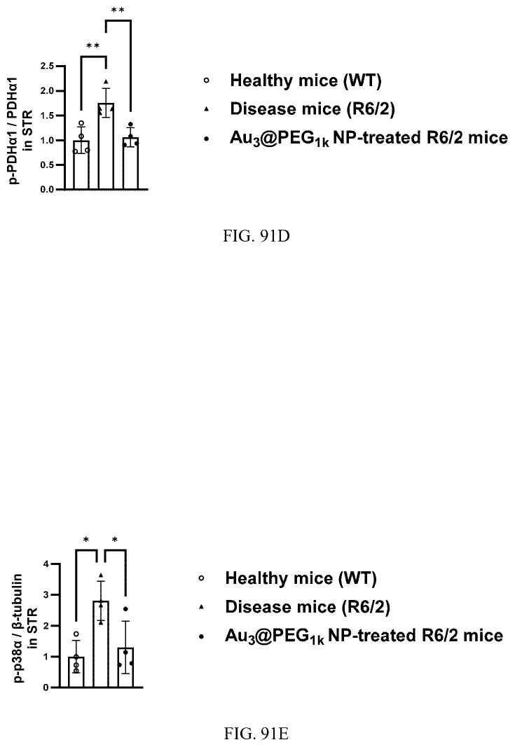

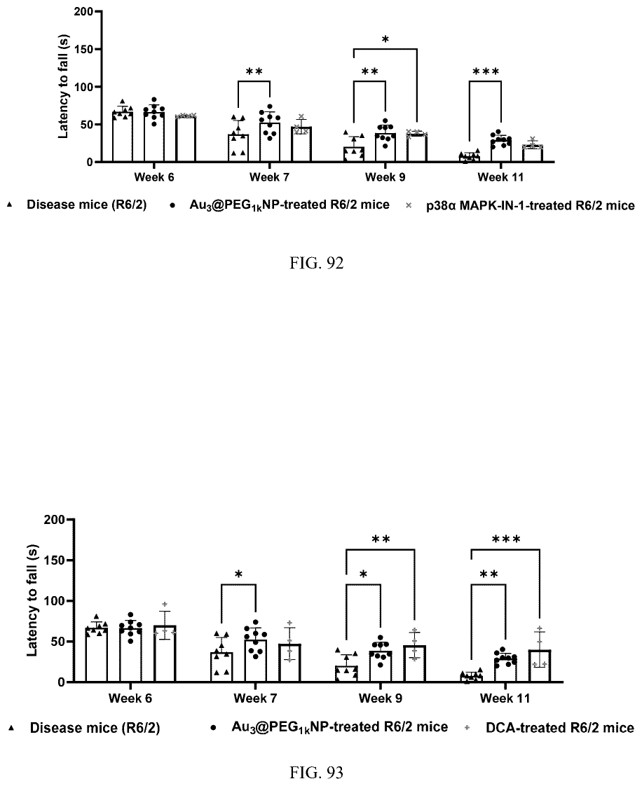

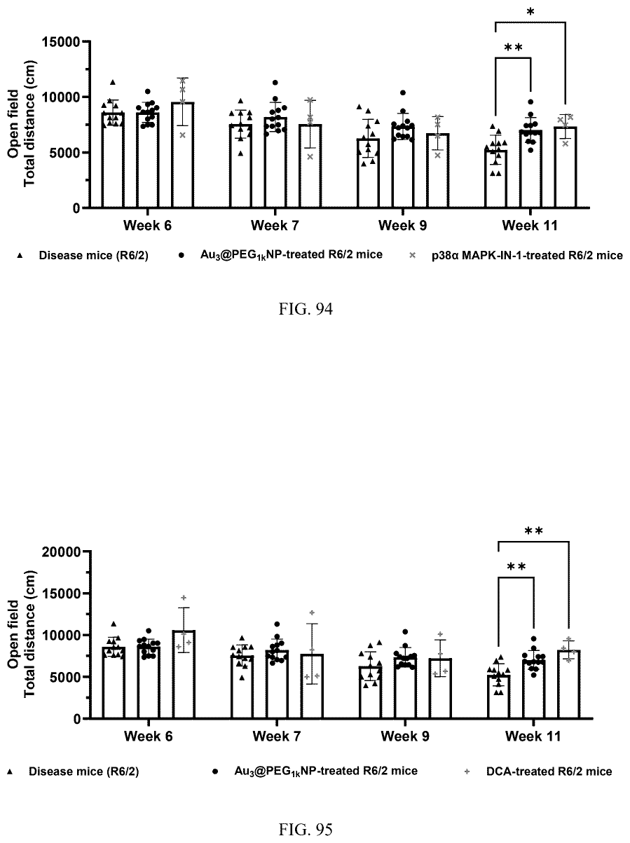

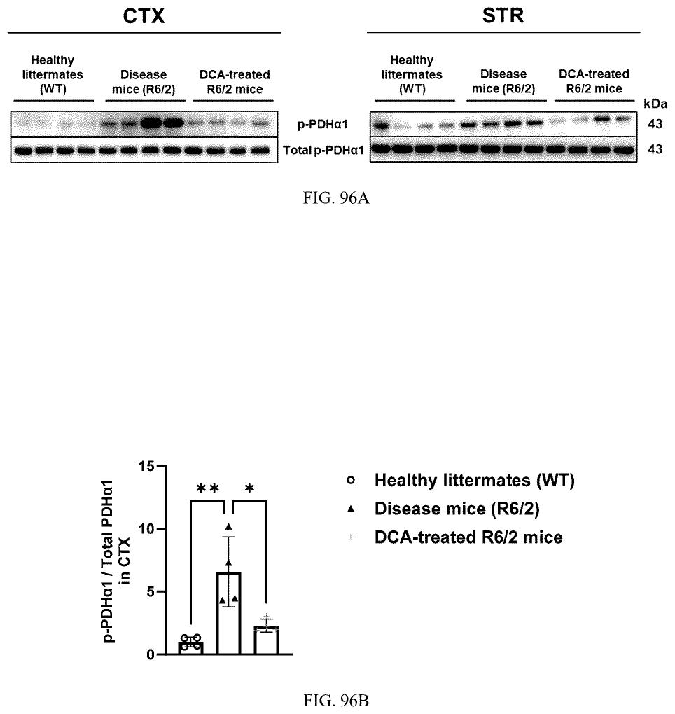

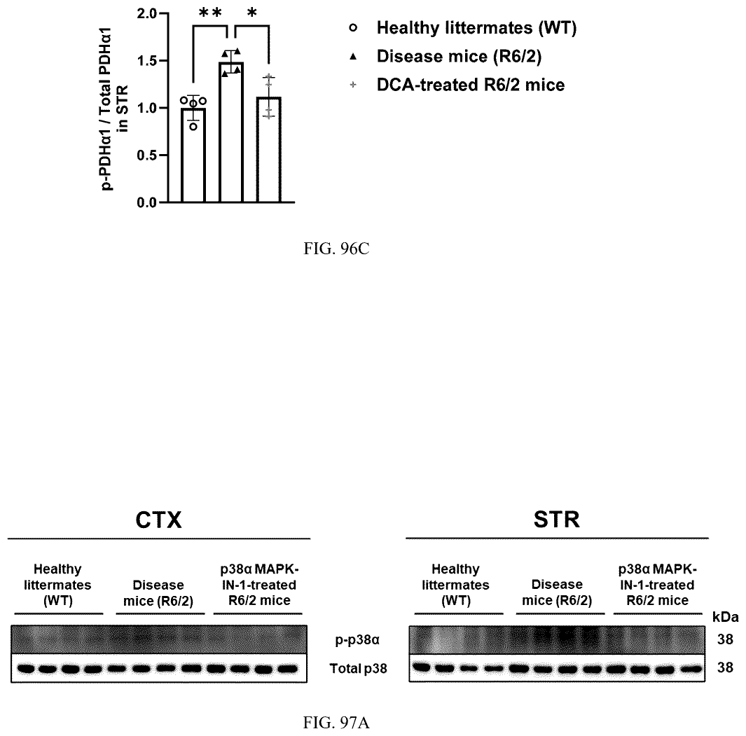

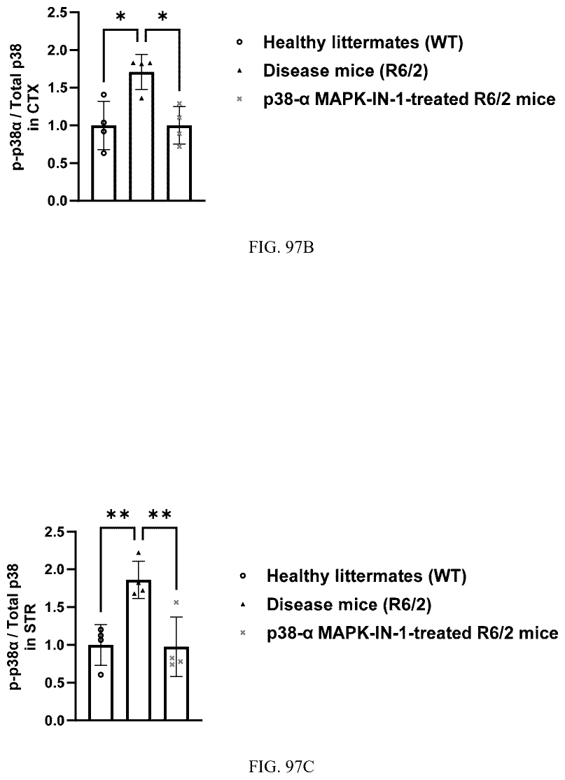

To validate the kinome screening data in vivo, we employed western blot to prove that Au₃@PEG₁ₖ NP treatment (as outlined in FIG. 51) inhibited p-p38α and p-PDH, a downstream target of PDK1, in both cortex and striatum of R6/2 HD mice (FIGS. 91A-91E). This result matched report of p-p38α inhibition by gold NPs in the kidneys [66]. Moreover, to confirm the therapeutic roles of p38α and PDK1 in HD, we i.p. injected their respective pharmacological inhibitors, p38α MAPK-N-1 and dichloroacetate, daily into R6/2 mice and monitored their motor behavior. The rotarod test showed improved motor behavior at the age of Week 9 only for p38α MAPK-N-1-treated mice (FIG. 92) and at the age of Weeks 9 and 11 for dichloroacetate-treated mice (FIG. 93). The open field test showed that longer total walking distances for both p38α MAPK-N-1-and dichloroacetate-treated mice (FIGS. 94 and 95). Western blot further confirmed the inhibition of p-p38α and p-PDH by p38α MAPK-N-1 and dichloroacetate treatments, respectively, in the cortex and striatum (FIGS. 96 and 97). In conclusion, p-p38α and PDK1 are therapeutic targets of Au₃@PEG₁ₖ NP in the HD brain.

### EXAMPLE 5

### Inhibition of Pyroptosis-Mediated Cell Death

As HD is a neurodegenerative disease, we reasoned that Au₃@PEG₁ₖ NP exerts its anti-

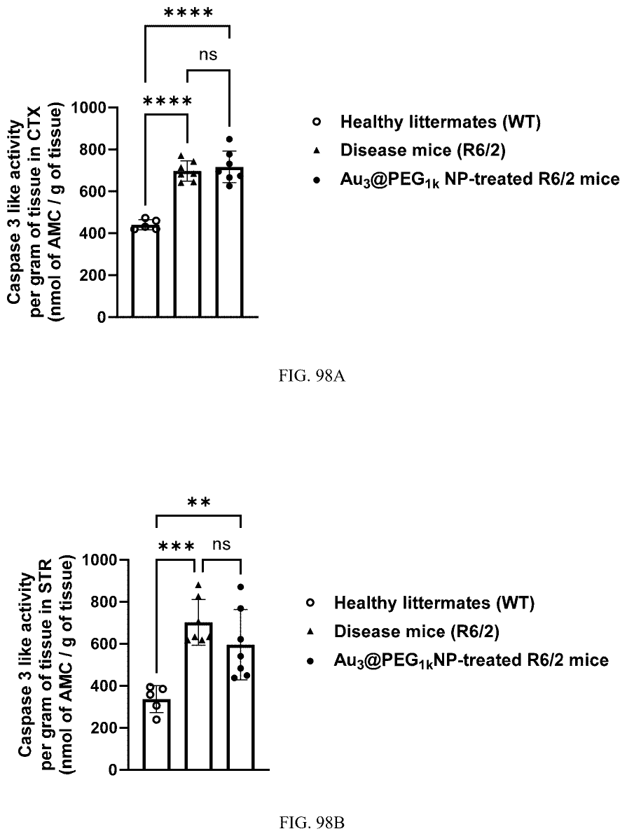

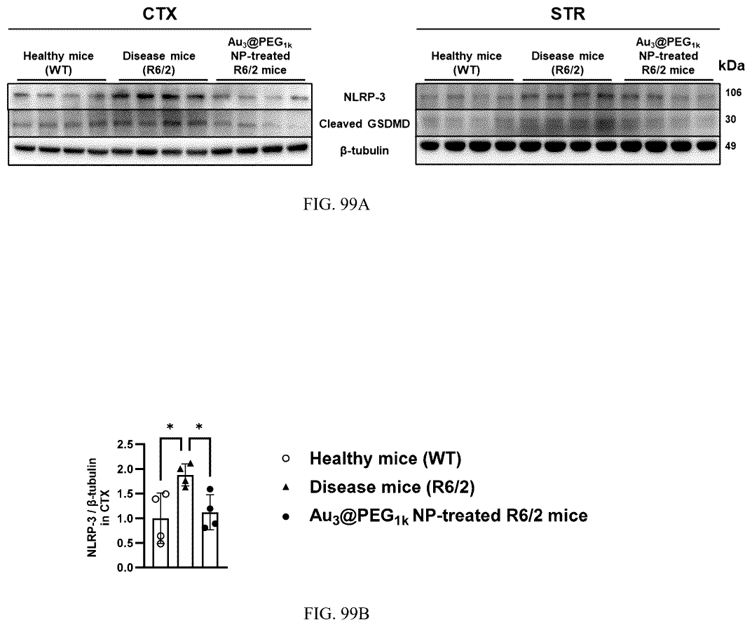

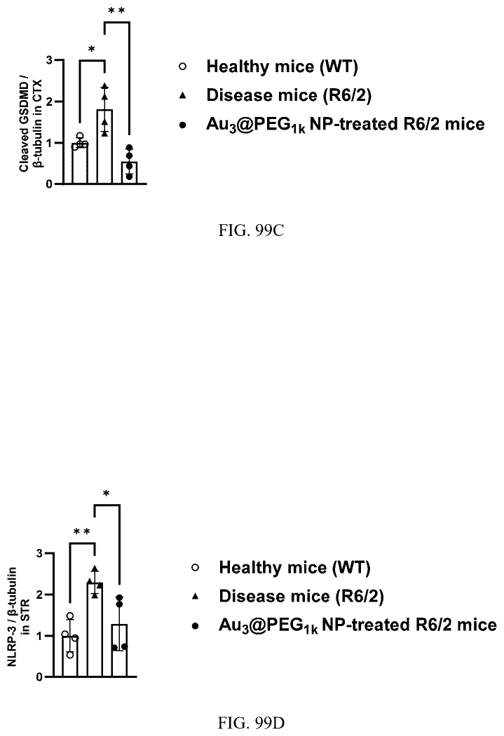

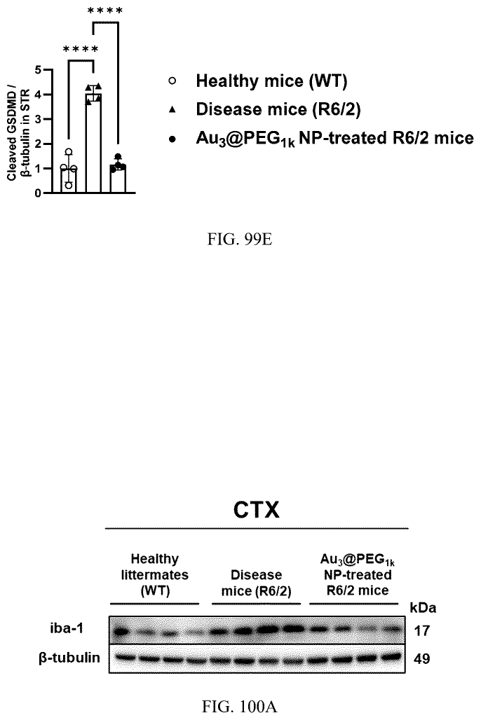

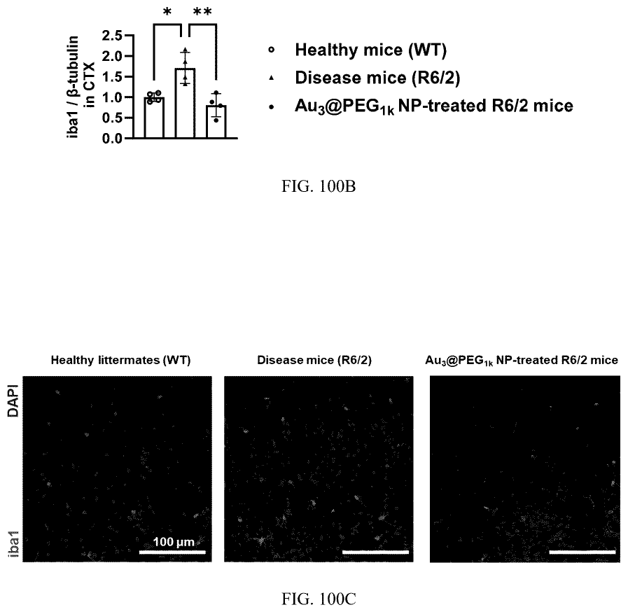

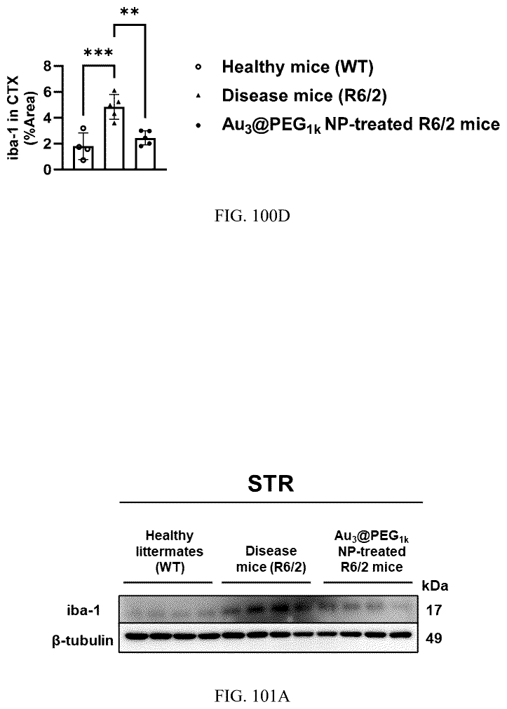

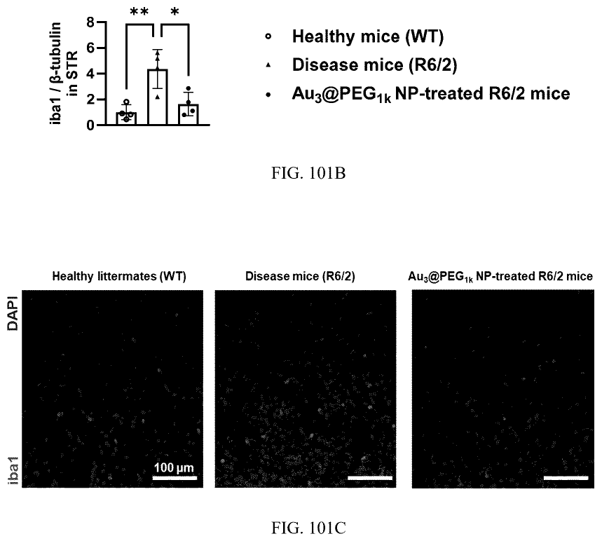

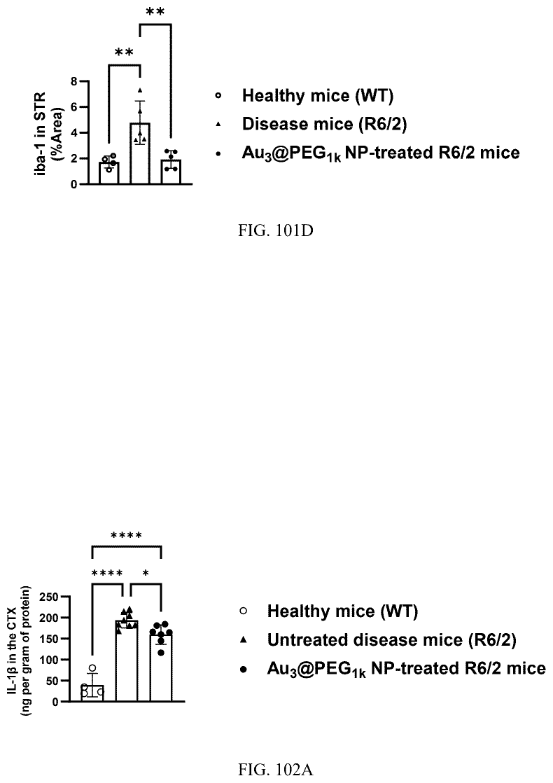

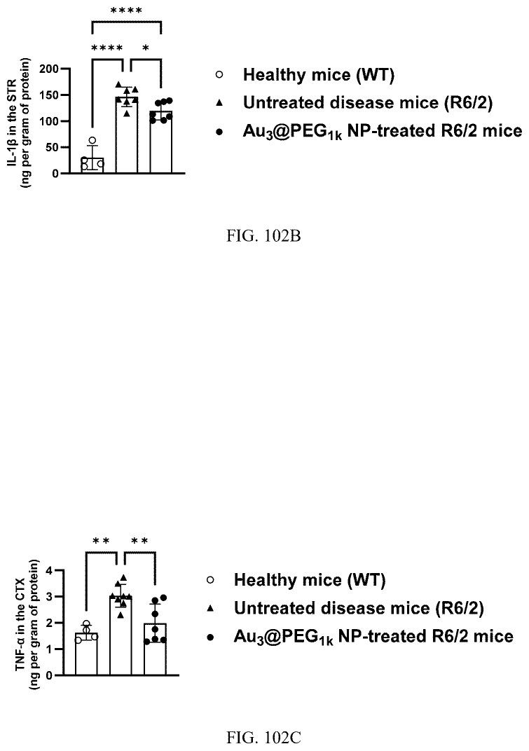

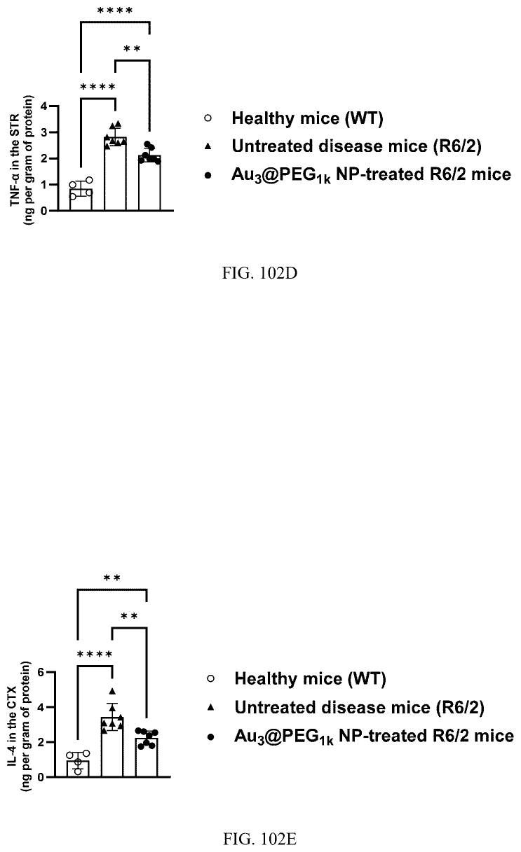

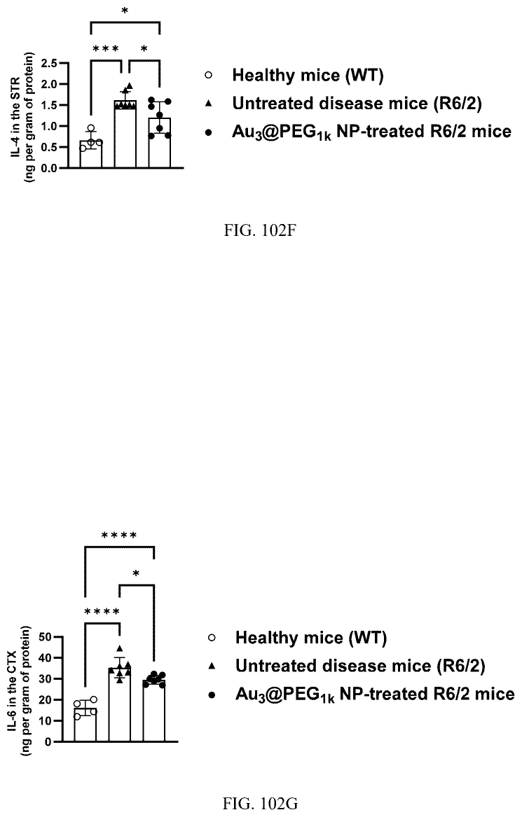

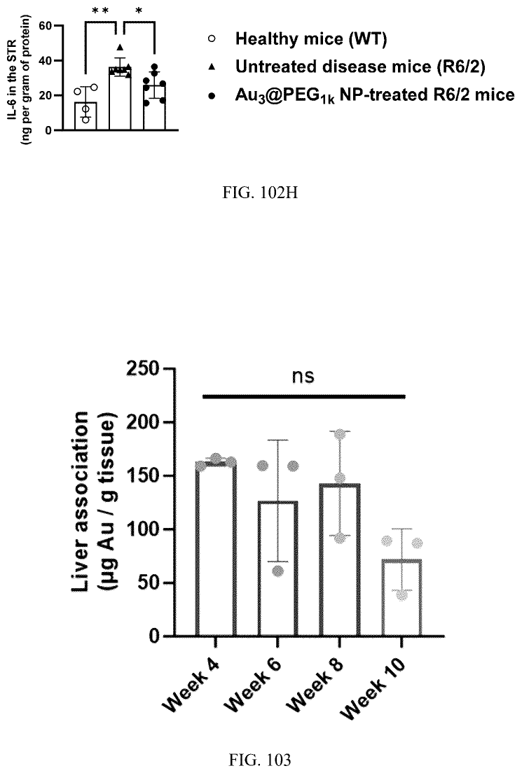

HD efficacy by suppressing pathways related to cell death [67]. Firstly, we tested the involvement of apoptosis, a classical pathway that entails a cascade of caspase activation to mediate signal transduction and cellular destruction. Yet, Au₃@PEG₁₀₀₀ NP treatment (based on FIG. 51) did not lead to inhibition of the activity of cleaved caspase-3 in the HD brain of R6/2 mice, suggesting the ruling out of apoptosis (FIGS. 98A and 98B). Next, we considered the role of pyroptosis, an emerging pathway of cell death in HD pathogenesis [68, 69] whereby activated caspases cleave gasdermins to form pores in the cell membrane. Western blot analysis of the striatum and cortex of R6/2 mice revealed the downregulation of nucleotide-binding domain, leucine-rich-containing family, pyrin domain-containing protein (NLRP)-3 and gasdermin D [70] following Au₃@PEG₁ₖ NP treatment (FIGS. 99A-99E), evidence of inhibited pyroptosis-mediated cell death in the HD brain. This result matches the recently reported role of PDK1 inhibition in reducing pyroptosis [71]. Because NLRP-3 inflammasome and PDK1 activities trigger the secretion of proinflammatory cytokines (and pyroptosis cell death) [72-74], we also verified that Au₃@PEG₁ₖ NP treatment reduced inflammation in the striatum and cortex of the HD brain, based on: (i) western blot to prove the inhibition of iba1, (ii) confocal immunofluorescence to visualize an attenuated area of microglial cells (FIGS. 100 and 101), and (iii) enzyme-linked immunosorbent assay (ELISA) to show the inhibition of cytokine markers of HD [75], including interleukin (IL)-1B, tumor necrosis factor (TNF)-a, IL-4, and IL-6 (FIGS. 102A-102H). Collectively, these data indicate that Au₃@PEG₁ₖ NP reduced pyroptosis and neuroinflammation in the HD brain, consistent with a past report on leveraging PDK1 inhibition for alleviating neuroinflammation [76].

We showed that 11-nm Au₃@PEG₁ₖ NPs selectively accumulated in the HD brain over healthy brain and enter neurons and microglia in the cortex and striatum. The key innovation lied in defining the NP size at 10-20 nm for circumventing three biological barriers that have long obstructed brain delivery: renal clearance, liver sequestration, and brain barrier penetration. Adding targeting ligands promoted specific delivery to neurons and microglia, but would enlarge the NP size and risk liver sequestration. NP surface chemistry might be another key parameter for brain delivery. Lipka et al. i.v. injected three gold NP types into healthy rats, but they observed inefficient brain delivery [88]. The phosphine-capped gold NP was similarly sized as Au₃@PEG₁ₖ NP (11 nm), but lacked a PEG coating to extend blood circulation and prevent liver clearance [both factors favoring brain delivery; [89]]. Au₅@PEG₇₅₀ NP (21 nm) is larger than our Au₃@PEG₁ₖ NP as its PEG coating contains dodecane and poly (maleic anhydride), but it is unclear if both coating components cause liver clearance and restrict brain delivery. Au₅@PEG_{10K} NP (31 nm), while also containing both coating components, showed improved blood circulation and reduced liver uptake due to its longer PEG chains, but its larger NP size disfavors brain delivery. In absolute mass, the accumulation of our Au₃@PEG₁ₖ NP in the HD brain (0.71 μg/g) is orders of magnitude higher than that of Au₅@PEG_{10K} NP in the healthy brain as reported by Xu et al., [90] possibly due to a smaller NP size for prolonged blood circulation, absence of non-PEG coating components, HD pathology that favors brain delivery, and a ˜200-fold higher gold NP dose to mitigate liver clearance in terms of percent injected dose (% ID) (FIG. 103) and achieve therapeutic efficacy; our total gold dose was similar to that of chiral gold NP for treating Alzheimer's disease (˜100 mg-gold/kg-mouse) [92].

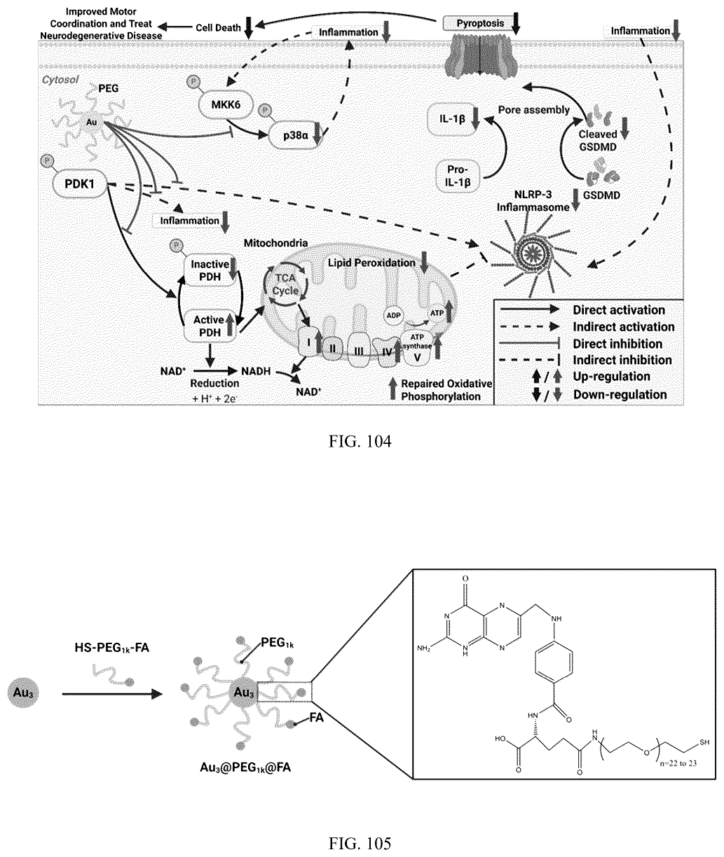

Another major finding is that Au₃@PEG₁ₖ NPs are self-therapeutic agents for HD; no chemical drugs, biologics, or physical forces are needed (FIG. 104).

### EXAMPLE 6

### Loading Small Molecule Drug on 11-nm Gold Nanoparticle Can Improve Efficacy in Huntington'S Disease

Oxidative stress is an essential cause for progressive neurodegeneration and, accordingly, antioxidants are an attractive approach to reducing oxidative stress in neuronal cells and HD progression. Vitamins C and D prevent the progression of postural instability in HD patients. Coenzyme Q10 (CoQ10) and vitamin E show neuroprotective properties by reversing loss in electron transport activity induced by nitropropionic acid (3-NP) in rat models of HD [77]. Interestingly, folic acid (FA; Vitamin B9) is seldom used for treating HD even though it alleviates symptoms of other neurodegenerative diseases, such as AD. FA deficiency increases the risk for AD, whereas its sufficient intake protects against Alzheimer's disease [78]. HD patients have higher levels of homocysteine (due to lower levels of folate) than healthy subjects, so HD patients have higher neopterin levels that are correlated to increased immune activation [79]. As a linkage between FA and HD, previous study showed that intraperitoneally injection of FA to rats pretreated with 3-NP counteracted the free radicals generated by 3-NP, but it remains uncertain if FA effectively treats HD [80]. Because small molecules like FA face renal clearance, we postulated that the attachment of FA molecules, as the model small molecule to self-therapeutic, anti-inflammatory gold NPs that enter the HD brain would empower FA to show their antioxidizing efficacy in the brain.

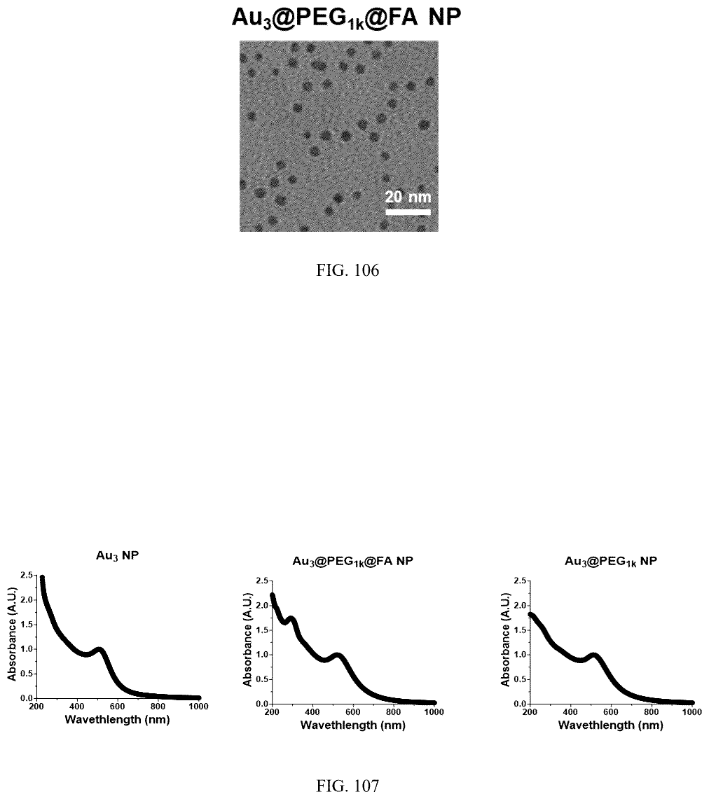

After loading the thiolated PEG strands conjugated with FA on the Au NP (FIG. 105), the NPs were characterized (FIGS. 106 and 107 and Table 6) and TEM imaging showed similar core size as unmodified Au₃ NP and Au₃@PEG₁ₖ NP.

TABLE 6
Physicochemical properties of the NPs.

| | | Hydrodynamic | ζ potential | |
|---|---|---|---|---|
| | Physical diameter | diameter (nm) in | (mV) in 1 mM | Polydispersity |
| Nanoparticles | of Au core (nm) ^{a} | water at RT ^{b} | KCl at RT | index |
| Au₃ NP | 3.3 ± 0.6 | 5.5 ± 0.6 | −27.9 ± 15.0 | 0.01 ± 0.01 |
| Au₃@PEG₁ₖ NP | 3.0 ± 0.5 | 10.7 ± 0.7 | −9.6 ± 0.5 | 0.03 ± 0.02 |
| Au₃@PEG₁ₖ@FA NP | 2.7 ± 0.5 | 9.7 ± 0.5 | −13.3 ± 2.1 | 0.09 ± 0.02 |

^{a} Physical diameter of the gold core was measured from TEM images.
^{b} Hydrodynamic diameters, polydispersity indice (PDI), and zeta (ζ) potentials were measured by dynamic light scattering (DLS) at room temperature (RT). X = diameter of Au core (nm); y = molecular weight of PEG (Da); PDI = polydispersity index.
All reported data represent mean ± SD from three independent measurements.

UV-vis showed plasmonic peaks around 500 nm corresponding to Au core while the presence of the peaks around 280 nm corresponds to the successful conjugation of FA molecules on the Au NP. DLS showed similar size of Au₃@PEG₁ₖ@FA NP as Au₃@PEG₁ₖ NP and the zeta potential of the former NP was slightly more negative because the FA molecule is negative in charge. By fluorescence based-direct displacement of thiolated PEG strands, the loading density of SH-PEG₁ₖ-FA on each Au NP is 29.0±5 strands.

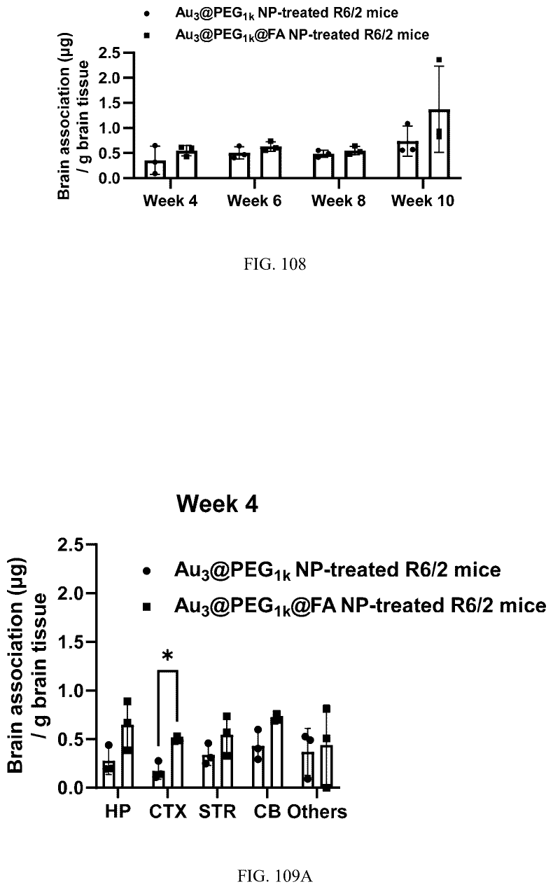

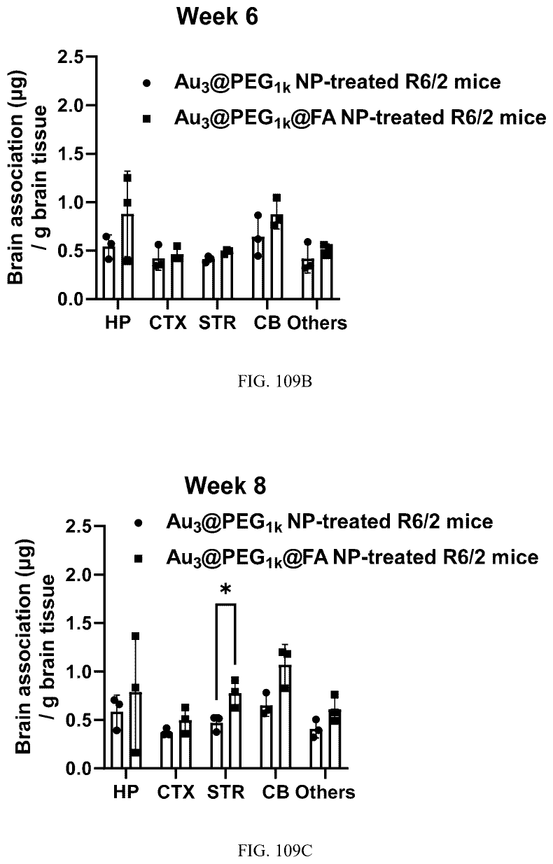

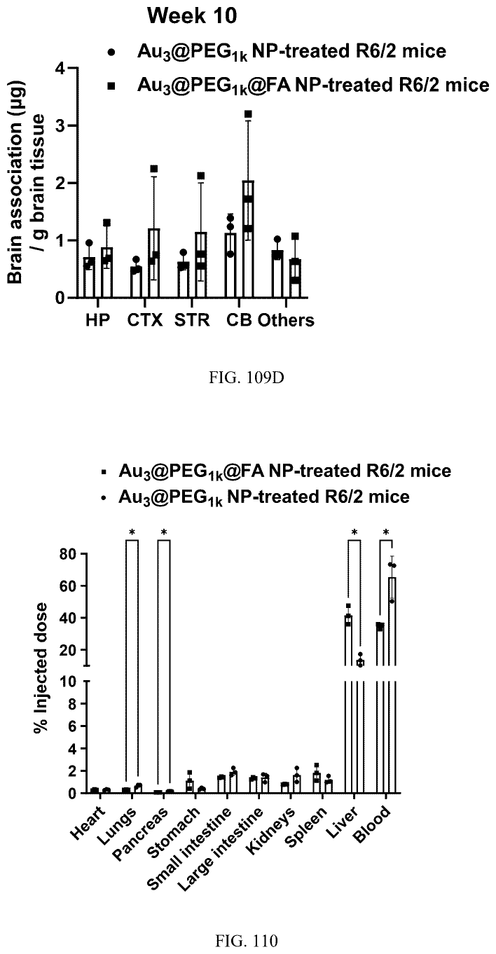

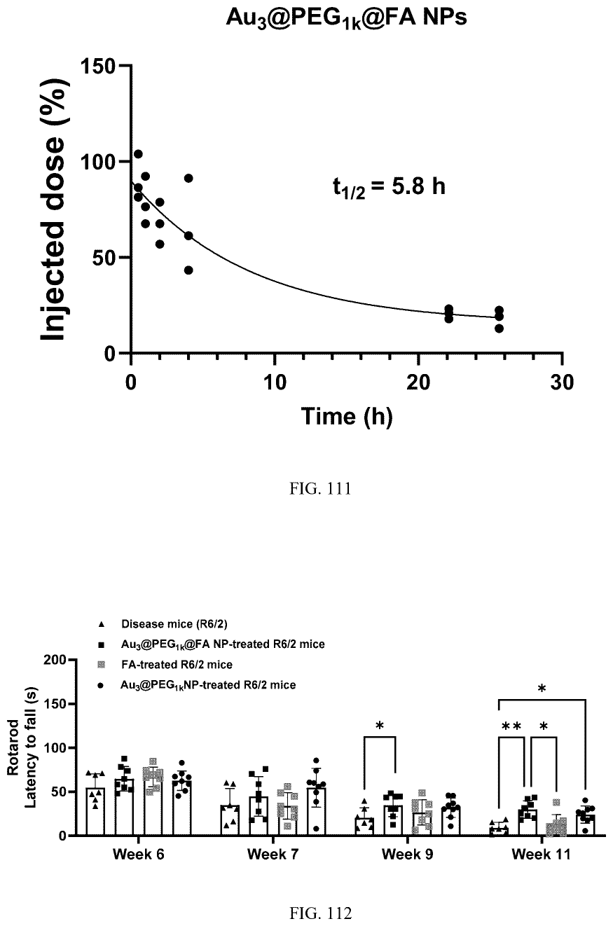

The as-prepared Au₃@PEG₁ₖ @FA NP was then i.v. injected into R6/2 mice of different ages. 24 h post injection, the brains were harvested and acid-digested, subjecting to ICP-MS measurement. Results showed no significant enhancement in brain uptake across all the Weeks of ages (FIG. 108). However, the mean brain association of the Au₃@PEG₁ₖ@FA NP-treated R6/2 mice was consistently higher than that in Au₃@PEG₁ₖ NP across all the Weeks of ages. Looking into different brain regions, we observed significant enhancement of Au association in cortex of Week 4 R6/2 mice and striatum of Week 8 R6/2 mice (FIGS. 109A-109D). At the same time, other organs of Week 10 R6/2 mice were harvested for ICP-MS measurement which revealed significantly higher liver and spleen uptake in Au₃@PEG₁ₖ@FA NP-treated group (FIG. 110). As a result, the blood half-life of the Au₃@PEG₁ₖ@FA NP-treated R6/2 mice (FIG. 111; ˜6 h) was much shorter than Au₃@PEG₁ₖ NP-treated R6/2 mice (FIG. 9; ˜35 h). We concluded that Au₃@PEG₁ₖ@FA NP could achieve similar brain association as Au₃@PEG₁ₖ NP with shorter blood half-life. This may be attributed to the presence of folate receptors on the brain endothelial cells and choroid plexus [81-85].

We evaluated the therapeutic efficacy of Au₃@PEG₁ₖ@FA NP in R6/2 HD mice. Following the same treatment schematic as the previous study (FIG. 51), each R6/2 mice was i.v. injected with 84 nmol of free FA (equivalent dose of folate on the Au₃@PEG₁ₖ@FA NP) or 700 μg of Au₃@PEG₁ₖ@FA NP (equivalent dose of Au NP as Au₃@PEG₁ₖ NP). Utilizing the rotarod test, we observed delayed motor impairment in Au₃@PEG₁ₖ@FA NP-treated mice relative to untreated mice as early as Week 8 (FIG. 112). Notably, Au₃@PEG₁ₖ@FA NP prolonged the residency on the rotarod (or reduced their latency to fall off) relative to untreated mice with a more significant therapeutic efficacy to Au₃@PEG₁ₖ NP, but free FA did not show improvement at this dosing regimen.

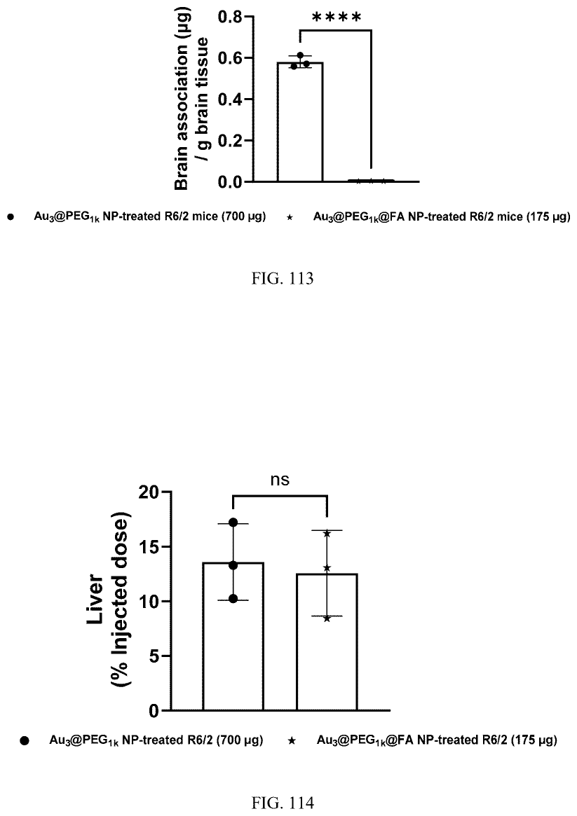

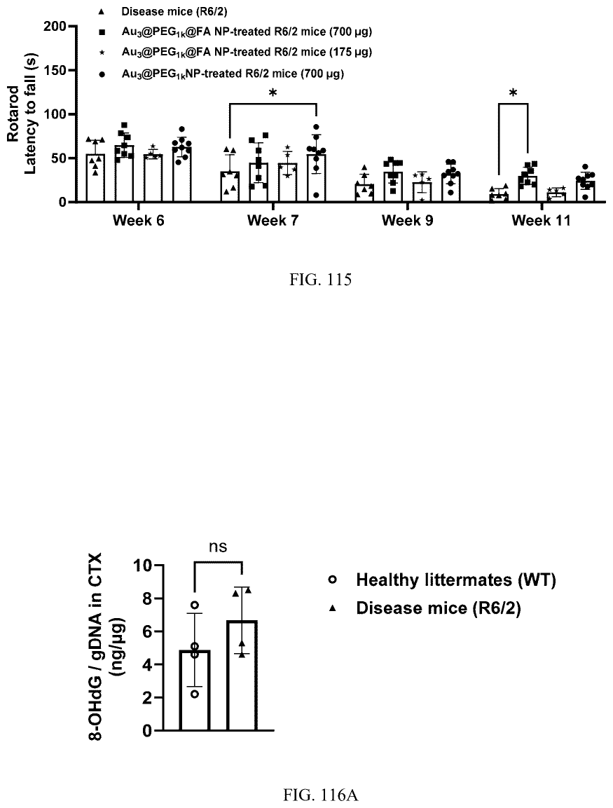

As there is a high percentage injected dose of Au₃@PEG₁ₖ@FA NP in the liver (FIG. 110), we tried to decouple the therapeutic effects of liver uptake of Au₃@PEG₁ₖ@FA NP on improving latency to fall by rotarod test by keeping the similar liver uptake of NPs and keeping the brain from NPs. Therefore, 175 μg of Au₃@PEG₁ₖ@FA NP (4-fold lower than the original dose) was i.v. injected to Week 10 R6/2 mice. By ICP-MS measurement, significantly low brain uptake of Au₃@PEG₁ₖ@FA NP relative to Au₃@PEG₁ₖ NP was observed (FIG. 113) while keeping similar liver uptake in the mice (FIG. 114). Next, we applied 175 μg of Au₃@PEG₁ₖ@FA NP to R6/2 mice following the treatment plan as before (FIG. 51). Rotarod test did not reveal significant improvements in latency to fall across all the weeks tested (FIG. 115), suggesting that the therapeutic effects mainly originated from the presence of NPs in the brain but not from liver uptake.

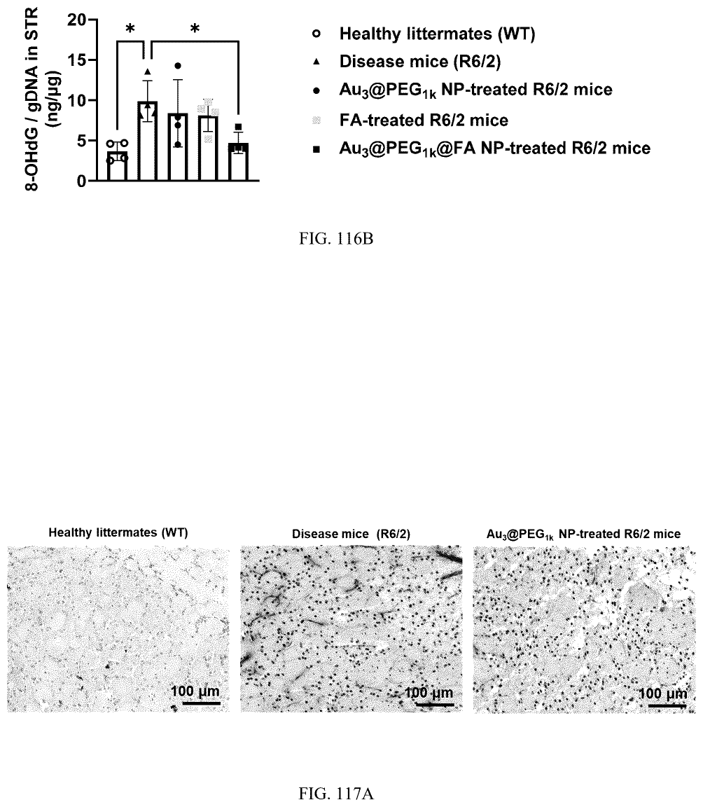

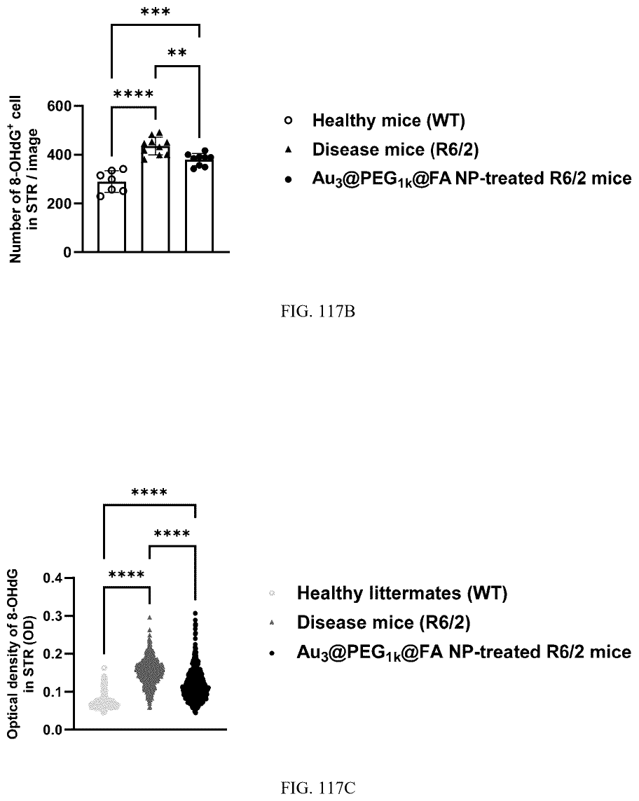

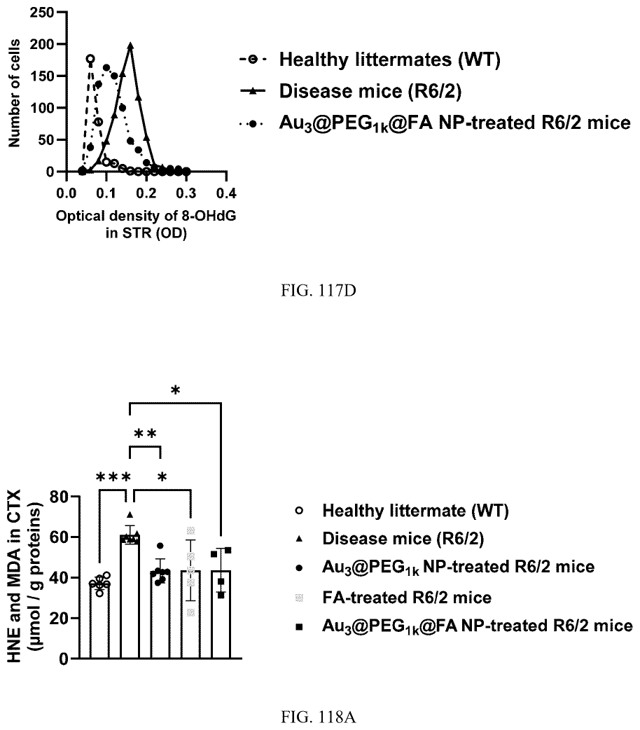

We tried to study the therapeutic effects contributed by FA on the Au NP. Previous study showed that deficiency would contribute to the reduced expression of NUDT15 which leads to DNA damage as indicated by increased in the level of 8-OHdG in DNA [86]. Therefore, we measured the level of 8-OHdG in the primarily affected regions which are cortex and striatum of the R6/2 mice. Consistent to the literature, the [87] level of the 8-OHdG was not upregulated significantly in the cortical regions of the R6/2 mice (FIG. 116A), but there was an increase in the level of the 8-OHdG in the striatal regions as compared to WT mice (FIG. 116B). Upon 5 weeks of i.v. injection of Au₃@PEG₁ₖ@FA NP to R6/2 mice as the treatment plan (FIG. 51), 8-OHdG ELISA assay revealed a significant reduction of 8-OHdG level in striatum of Au₃@PEG₁ₖ@FA NP-treated group compared to untreated R6/2 mice while there was no significant drop in 8-OHdG level in free FA-treated or Au₃@PEG₁ₖ NP-treated group (FIG. 116B). We reasoned that the therapeutic effects of Au₃@PEG₁ₖ@FA were higher at the chosen dosing regimen. Immunohistochemistry revealed that there was a lower number of 8-OHdG⁺ cells in the striatal regions of Au₃@PEG₁ₖ@FA NP-treated group (FIGS. 117A and 117B). By conducting semi-quantitative analysis of the same sets of immunohistochemical images, we noticed a reduction in population of striatal cells with higher optical density of 8-OHdG (FIGS. 117C and 117D). The reduction of 8-OHdG level can also be attributed to the alleviation of oxidative stress, therefore we also performed lipid peroxidation assay to measure HNE and MDA molecule which were increased in the cortex and striatum of R6/2 mice as compared to WT mice (FIG. 118). In cortex, results showed reduction in all the treatment group including free FA-treated, Au₃@PEG₁ₖ@FA NP-treated, and Au₃@PEG₁ₖ NP-treated R6/2 mice. In striatum, only Au₃@PEG₁ₖ@FA NP-treated and Au₃@PEG₁ₖ NP-treated groups showed reduction in lipid peroxidation, where more significant reduction as such was observed in Au₃@PEG₁ₖ@FA NP treatment compared to that of Au₃@PEG₁ₖ NP treatment. In short, Au₃@PEG₁ₖ@FA has higher capabilities in both participating in DNA repairment or reduction in DNA damage by reducing oxidative stress.

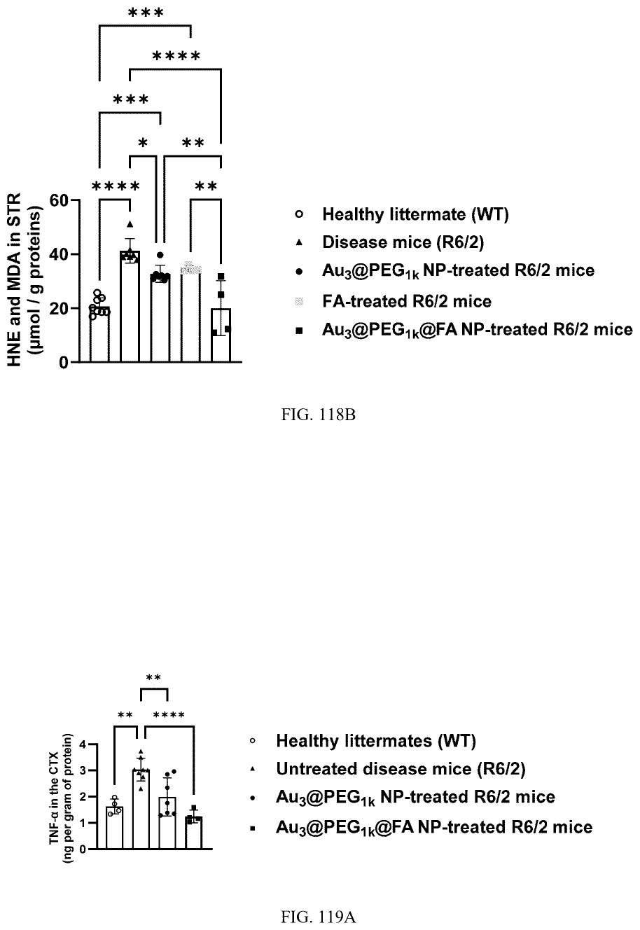

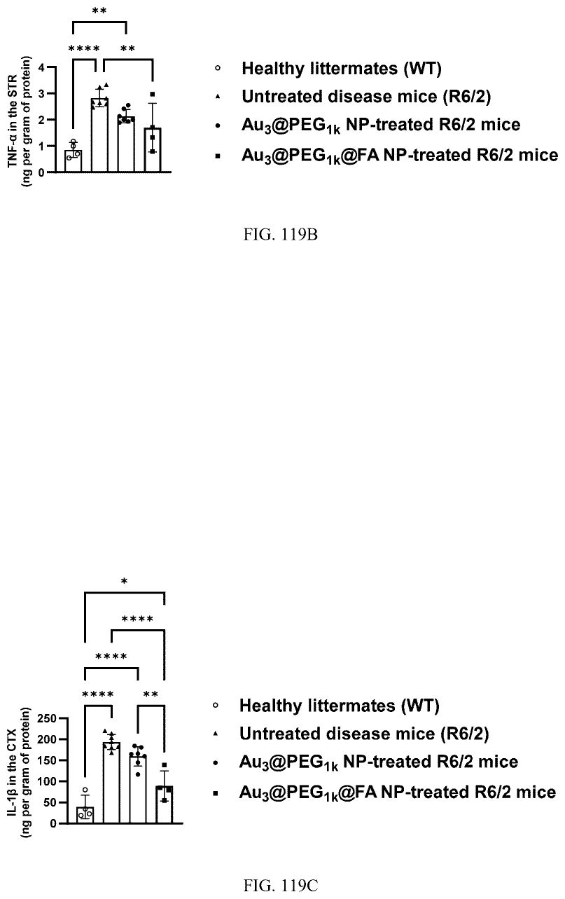

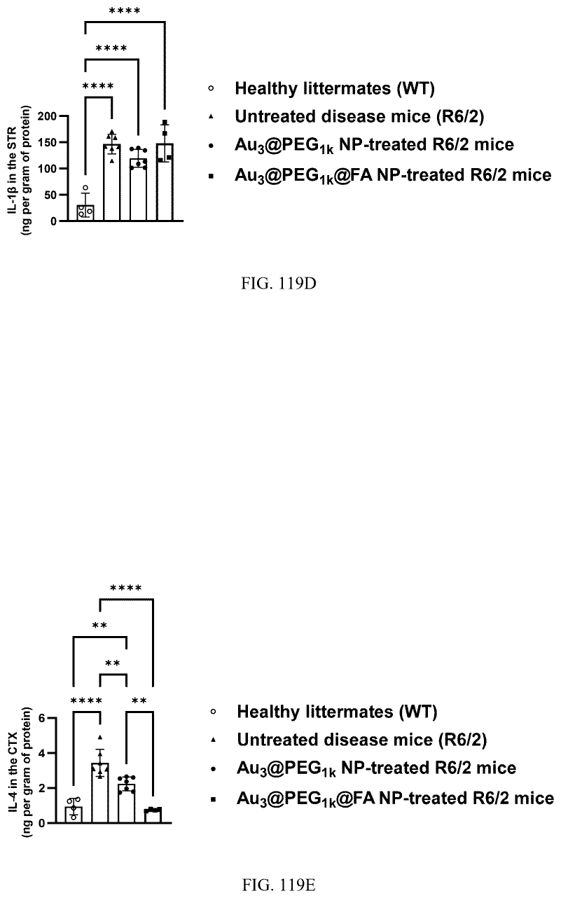

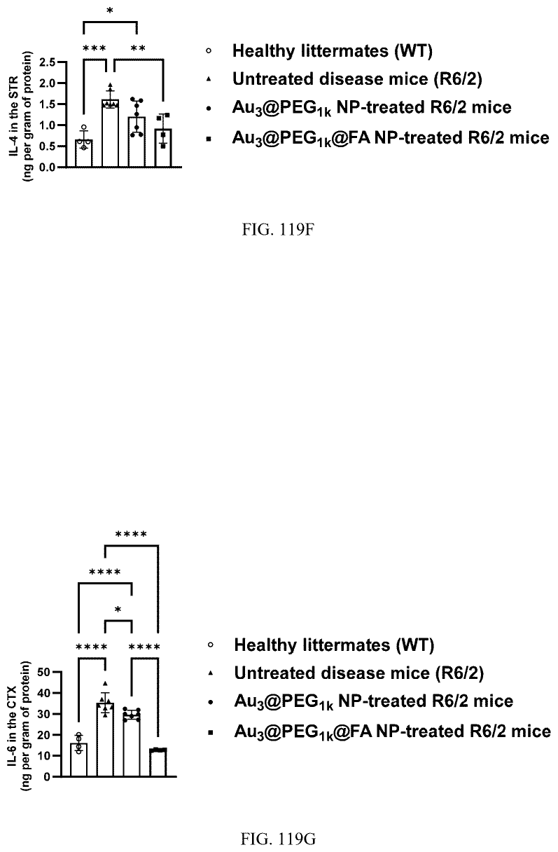

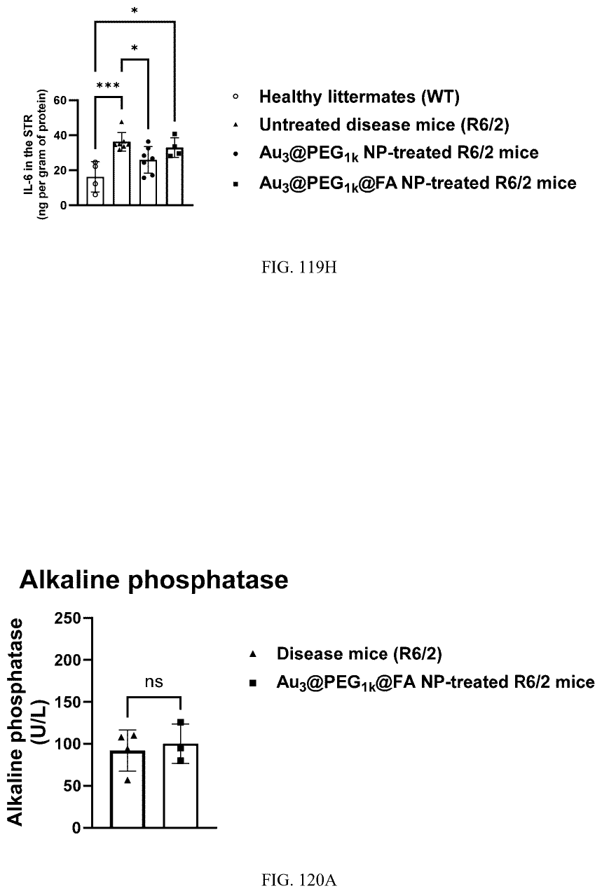

We further evaluated the role of FA on the Au NP in combating neuroinflammation. Following 5 i.v. injection, ELISA showed reduced TNF-α, IL-1β, IL-4, and IL-6 in cortex and striatum of Au₃@PEG₁ₖ@FA NP-treated R6/2 mice except striatal IL-1β and IL-6 level (FIGS. 119A-119H). We reasoned that the addition of FA might have biased the NP to some particular anti-inflammation activities slightly. The Au₃@PEG₁ₖ@FA NP treatment outperformed the Au₃@PEG₁ₖ NP-treated group in most situations, indicating the additional anti-neuroinflammation effects of FA on the Au NP.

We tested the systemic toxicity upon Au₃@PEG₁ₖ@FA NP of R6/2 mice 5 post i.v.

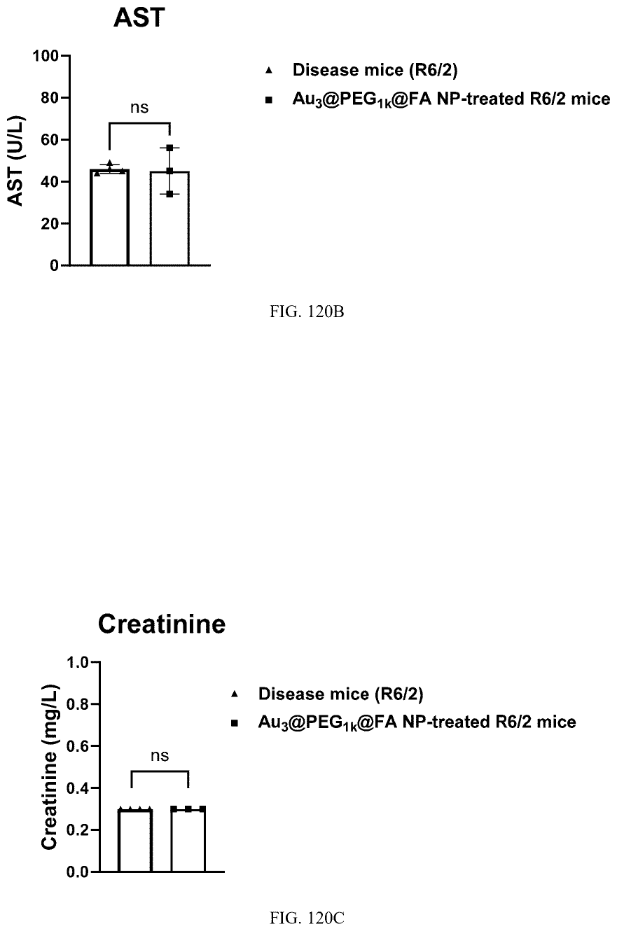

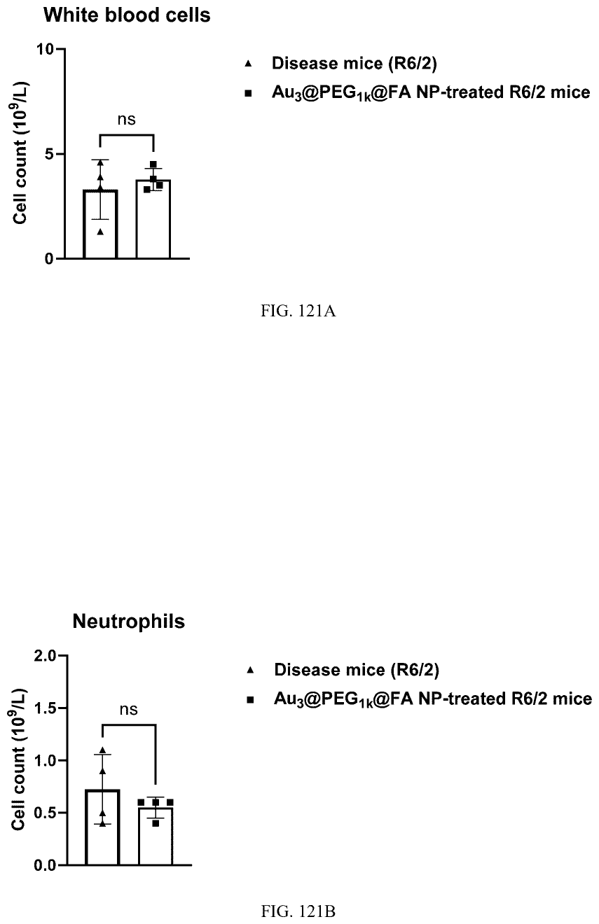

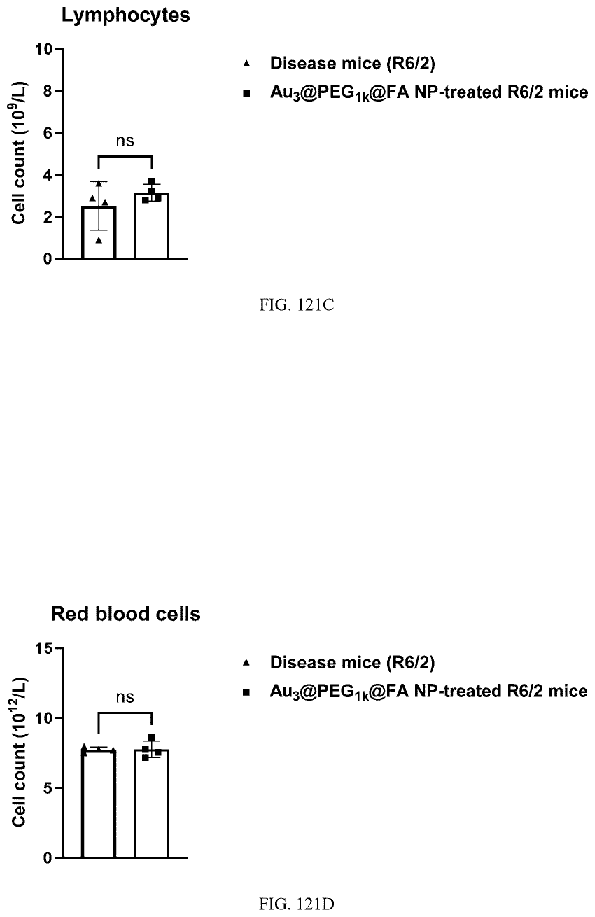

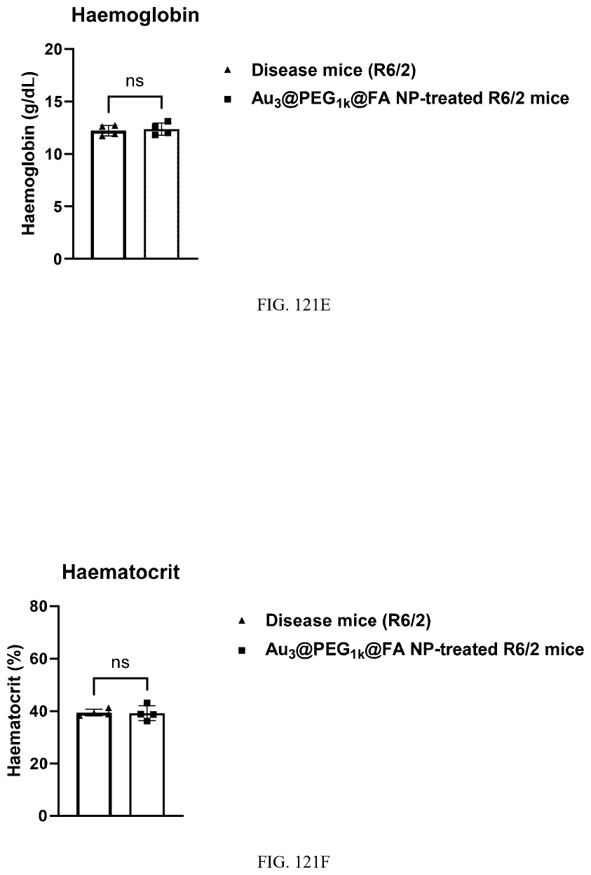

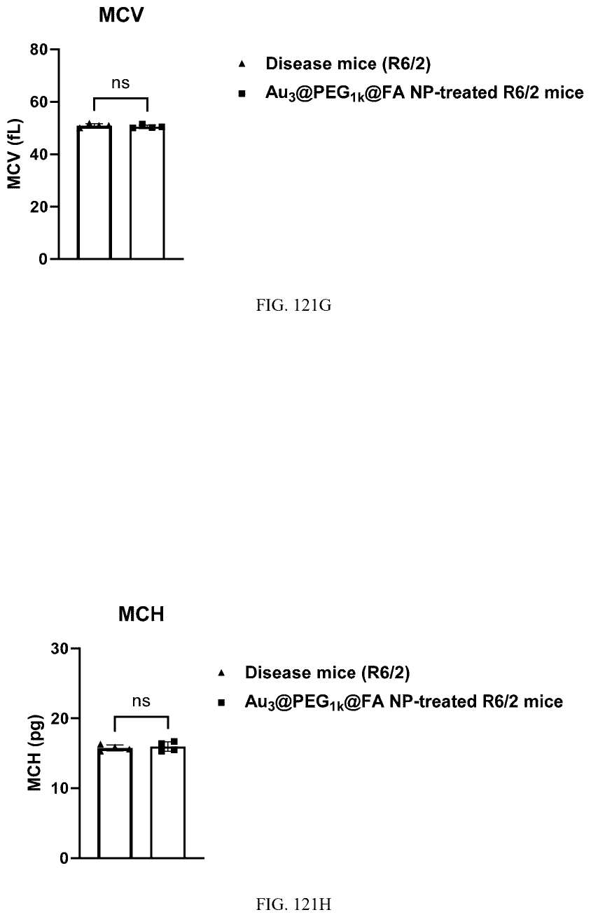

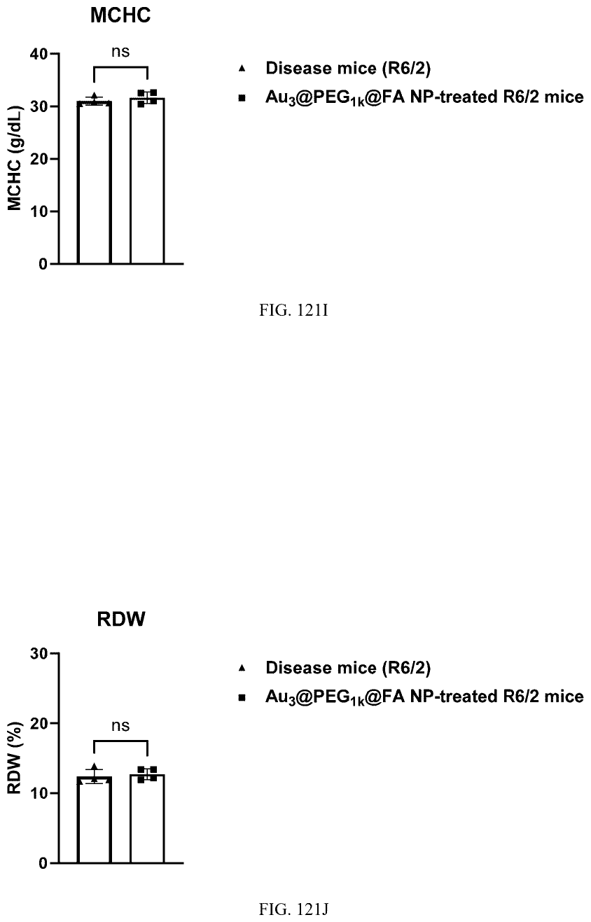

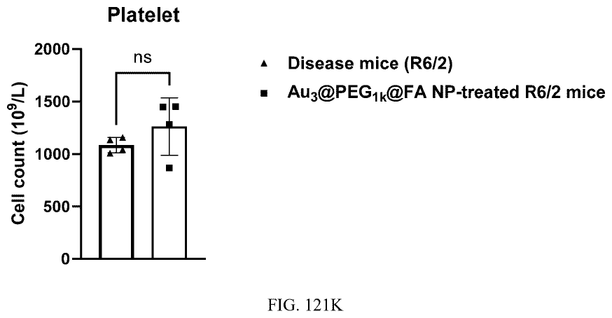

injections. Biochemical analysis of serum showed no change in alkaline phosphatase, aspartate aminotransferase (AST), indicating limited toxicity in liver while no changed in the levels in blood creatinine suggested normal kidney function (FIGS. 120A-120C). Hematology analysis did not show any changes in white blood cells, red blood cells, and platelets (FIGS. 121A-121K). In a nutshell, there is limited evidence of systemic toxicity upon the treatment of Au₃@PEG₁ₖ@FA NP.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### Exemplary Embodiments

Embodiment 1 A self-therapeutic composition for treating Huntington's disease (HD), the self-therapeutic composition comprising a drug carrier adapted for crossing the blood-brain barrier (BBB) for delivery in an extracellular brain space of a subject and for inhibiting renal or liver clearance in the subject, wherein the drug carrier comprises a metal nanoparticle (NP), wherein the nanoparticle comprises a metal core and a coating comprising a hydrophilic polymer and optionally a therapeutic agent comprising a linker conjugated to the hydrophilic polymer, wherein the linker comprises functional groups for binding to the metal core and to the polymer.
Embodiment 2. The composition of embodiment 1, wherein the metal NP comprises a gold core and wherein the hydrophilic polymer coating comprises thiolated polyethylene glycol (PEG) strands.
Embodiment 3. The composition of embodiment 2, wherein the metal NP is a (PEG)-coated gold NP comprising Au₃@PEG₁ₖ NP or Au₁₃@PEG₁ₖ NP.
Embodiment 4. The composition of embodiment 3, wherein the (PEG)-coated gold NP has a size of about from 10 to about 25 nm, wherein the gold core has a size of about from 2.7 to about 12.7 nm.
Embodiment 5. The composition of embodiment 3, wherein the gold core has a size of about 3-nm or about 13-nm, wherein the (PEG)-coated gold NP has a size of about 11-nm to 25-nm.
Embodiment 6. The composition of any preceding embodiment, wherein the PEG coating comprises not less than an average of 3.5 thiolated PEG strands per nm² of gold surface.
Embodiment 7. The composition of any preceding embodiment, wherein the PEG-coated gold NP strands are conjugated with Folic Acid (FA) to form Au₃@PEG₁ₖ@FA NP.
Embodiment 8. The composition of embodiment 7, wherein a loading density of SH-PEG₁ₖ-FA on each Au NP is about 29.0±5 strands.
Embodiment 9. A method for treating HD, the method comprising administering an effective amount of the composition of embodiment 1 to a subject with HD.
Embodiment 10. The method of embodiment 9, wherein the metal NP is Au₃@PEG₁ₖ NP or Au₁₃@PEG₁ₖ NP.
Embodiment 11. The method of embodiment 9, wherein the metal NP is Au₃@PEG₁ₖ@FA NP.
Embodiment 12. The method of embodiment 11, wherein the dosage of Au₃@PEG₁ₖ@FA NP administered to a subject ranges from about 0.1 mg/kg/day to about 0.5 mg/kg/day.
Embodiment 13. The method of embodiment 12, wherein the composition reduces oxidative stress and neuroinflammation by reducing 8-OHdG level in the striatum, TNF-α, IL-1β,IL-4, and IL-6 in cortex, and TNF-α and IL-4 in striatum of the subject.
Embodiment 14. The method of any preceding embodiment, wherein the composition is administered once weekly or monthly for about 5 years to about 10 years.
Embodiment 15. The method of any preceding embodiment, wherein the composition is administered intravenously (i.v.), via inhalation, transnasally, orally, sublingually, enterally, and/or parenterally.
Embodiment 16. The method of any preceding embodiment, wherein the subject is a mammal.
Embodiment 17. The method of embodiment 16, wherein the mammal is a human.
Embodiment 18. The method of embodiment 15, wherein the composition is administered intravenously for delivery into the cortex and striatum in a subject with HD.
Embodiment 19. The composition of embodiment 18, wherein the composition comprising Au₃@PEG₁ₖ NP reduces pyroptosis by inhibiting p38α mitogen-activated protein kinase (MAPK) phosphorylation and pyruvate dehydrogenase kinase 1 (PDK1) in the brain of the subject.
Embodiment 20. A method of synthesizing a gold nanoparticle for treating HD, the method comprising conjugating a gold NP with polyethylene glycol (PEG) and further conjugating a therapeutic agent with a linker to the PEG, wherein the gold nanoparticle is adapted for delivering the therapeutic agent to a therapeutic target.

### REFERENCES

1. Yano, H. et al. Inhibition of mitochondrial protein import by mutant huntingtin. Nat Neurosci 17, 822-831 (2014).
2. Solberg, O. K., Filkukovà, P., Frich, J. C. & Feragen, K. J. B. Age at Death and Causes of Death in Patients with Huntington Disease in Norway in 1986-2015. J. Huntingtons Dis. 7, 77-86 (2018).
3. Lee, L. K. C. et al. Preclinical Nanomedicines for Polyglutamine-Based Neurodegenerative Diseases. Mol Pharm. 18, 610-626 (2021).
4. Ferguson, M. W. et al. Current and Possible Future Therapeutic Options for Huntington's Disease. J Cent Nerv Syst Dis 14, 11795735221092516 (2022).
5. Meredith Wadman. Promising drug for Huntington disease fails in major trial. Science (2021).
6. Wu, J. R., Hernandez, Y., Miyasaki, K. F. & Kwon, E. J. Engineered nanomaterials that exploit blood-brain barrier dysfunction for delivery to the brain. Adv Drug Deliv Rev 197, 114820 (2023).
7. Zuckerman, J. E., Choi, C. H. J., Han, H. & Davis, M. E. Polycation-siRNA nanoparticles can disassemble at the kidney glomerular basement membrane. Proc. Natl. Acad. Sci. U. S. A. 109, 3137-3142 (2012).
8. Wiley, D. T., Webster, P., Gale, A. & Davis, M. E. Transcytosis and brain uptake of transferrin-containing nanoparticles by tuning avidity to transferrin receptor. Proceedings of the National Academy of Sciences 110, 8662-8667 (2013).
9. Thorne, R. G. & Nicholson, C. In vivo diffusion analysis with quantum dots and dextrans predicts the width of brain extracellular space. Proceedings of the National Academy of Sciences 103, 5567-5572 (2006).
10. Piella, J., Bastús, N. G. & Puntes, V. Size-Controlled Synthesis of Sub-10-nanometer Citrate-Stabilized Gold Nanoparticles and Related Optical Properties. Chemistry of Materials 28, 1066-1075 (2016).
11. FRENS, G. Controlled Nucleation for the Regulation of the Particle Size in Monodisperse Gold Suspensions. Nature Physical Science 241, 20-22 (1973).
12. Li, J. Y., Popovic, N. & Brundin, P. The use of the R6 transgenic mouse models of Huntington's disease in attempts to develop novel therapeutic strategies. NeuroRx 2, 447-464 (2005).
13. Pouladi, M. A., Morton, A. J. & Hayden, M. R. Choosing an animal model for the study of Huntington's disease. Nat Rev Neurosci 14, 708-721 (2013).
14. Ho, L. W. C., Yin, B., Dai, G. & Choi, C. H. J. Effect of Surface Modification with Hydrocarbyl Groups on the Exocytosis of Nanoparticles. Biochemistry 60, 1019-1030 (2021).
15. Benoit, D. N. et al. Measuring the Grafting Density of Nanoparticles in Solution by Analytical Ultracentrifugation and Total Organic Carbon Analysis. Anal Chem 84, 9238 (2012).
16. Kim, C. S. et al. Cellular imaging of endosome entrapped small gold nanoparticles. MethodsX 2, 306-315 (2015).
17. Kumar, P., Nagarajan, A. & Uchil, P. D. Analysis of Cell Viability by the Lactate Dehydrogenase Assay. Cold Spring Harb Protoc 2018, (2018).
18. Potts, E. M., Coppotelli, G. & Ross, J. M. Histological-Based Stainings using Free-Floating Tissue Sections. J Vis Exp (2020) doi: 10.3791/61622.
19. Dunnett, C. W. A Multiple Comparison Procedure for Comparing Several Treatments with a Control. J. Am. Stat. Assoc. 50, 1096-1121 (1955).
20. Z. Bao, et al., Prevention of age-related neuromuscular junction degeneration in sarcopenia by low-magnitude high-frequency vibration. Aging Cell 23, e14156 (2024).
21. E. Rizzuto, S. Pisu, A. Musarò, Z. Del Prete, Measuring neuromuscular junction functionality in the SOD1 (G93A) animal model of amyotrophic lateral sclerosis. Ann. Biomed. Eng. 43, 2196-2206 (2015).
22. J. A. Franco, H. E. Kloefkorn, S. Hochman, K. A. Wilkinson, An in vitro adult mouse muscle-nerve preparation for studying the firing properties of muscle afferents. J. Vis. Exp. e51948 (2014).
23. M. Odell, “Electric shocks and electrocution, clinical effects and pathology” in Encyclopedia of Forensic and Legal Medicine: Second Edition, (Elsevier, 2016), pp. 419-427.
24. S. P. Cairns, D. M. Robinson, D. S. Loiselle, Double-sigmoid model for fitting fatigue profiles in mouse fast-and slow-twitch muscle. Exp. Physiol. 93, 851-862 (2008).
25. K. E. Personius, R. P. Sawyer, Variability and failure of neurotransmission in the diaphragm of mdx mice. Neuromuscul. Disord. 16, 168-177 (2006).
26. del Pino, P. et al. Basic Physicochemical Properties of Polyethylene Glycol Coated Gold Nanoparticles that Determine Their Interaction with Cells. Angewandte Chemie International Edition 55, 5483-5487 (2016).
27. Walkey, C. D., Olsen, J. B., Guo, H., Emili, A. & Chan, W. C. W. Nanoparticle Size and Surface Chemistry Determine Serum Protein Adsorption and Macrophage Uptake. J Am Chem Soc 134, 2139-2147 (2012).
28. Yang, H. et al. Promoting intracellular delivery of sub-25 nm nanoparticles via defined levels of compression. Nanoscale 10, 15090-15102 (2018).
29. Peng, S. et al. CAG RNAs induce DNA damage and apoptosis by silencing NUDT16 expression in polyglutamine degeneration. Proc Natl Acad Sci U S A 118, (2021).
30. Suk, J. S., Xu, Q., Kim, N., Hanes, J. & Ensign, L. M. PEGylation as a strategy for improving nanoparticle-based drug and genedelivery. Adv Drug Deliv Rev 99, 28-51 (2015).
31. Nance, E. A. et al. A dense poly (ethylene glycol) coating improves penetration of large polymeric nanoparticles within brain tissue. Sci. Transl. Med. 4, 149ra119 (2012).
32. Jensen, S. A. et al. Spherical nucleic acid nanoparticle conjugates as an RNAi-based therapy for glioblastoma. Sci. Transl. Med. 5, (2013).
33. Valenza, M. et al. Cholesterol-loaded nanoparticles ameliorate synaptic and cognitive function in Huntington's disease mice. EMBO Mol Med 7, 1547-1564 (2015).
34. Spijker, S., Faliagkas, L. & Rao-Ruiz, P. Dissection of Rodent Brain Regions: Guided Free-Hand Slicing and Dissection of Frozen Tissue. in Neuroproteomics (ed. Li, K. W.) 7-19 (Springer New York, New York, NY, 2019). doi: 10.1007/978-1-4939-9662-9_2.
35. Liu, X., Miller, B. R., Rebec, G. V & Clemmer, D. E. Protein Expression in the Striatum and
Cortex Regions of the Brain for a Mouse Model of Huntington's Disease. J Proteome Res 6, 3134-3142 (2007).
36. Di Pardo, A. et al. Impairment of blood-brain barrier is an early event in R6/2 mouse model of Huntington Disease. Sci. Rep. 7, 41316 (2017).
37. Drouin-Ouellet, J. et al. Cerebrovascular and blood-brain barrier impairments in Huntington's disease: Potential implications for its pathophysiology. Ann Neurol 78, 160-177 (2015).
38. S. Zha, et al., Functionalized nanomaterials capable of crossing the blood-brain barrier. ACS Nano 18, 1820-1845 (2024).
39. R.-R. Xiao, et al., Constant-rate perfused array chip for high-throughput screening of drug permeability through brain endothelium. Lab Chip. 22, 4481-4492 (2022).
40. D. E. Tylawsky, et al., P-selectin-targeted nanocarriers induce active crossing of the blood-brain barrier via caveolin-1-dependent transcytosis. Nat. Mater. 22, 391-399 (2023).
41. X. Liu, B. R. Miller, G. V Rebec, D. E. Clemmer, Protein expression in the striatum and cortex regions of the brain for a mouse model of Huntington's disease. J. Proteome Res. 6, 3134-3142 (2007).
42. G. Danscher, Autometallography—A new technique for light and electron microscopic visualization of metals in biological tissues (gold, silver, metal sulphides and metal selenides). Histochemistry 81, 331-335 (1984).
43. D. T. Wiley, P. Webster, A. Gale, M. E. Davis, Transcytosis and brain uptake of transferrin-containing nanoparticles by tuning avidity to transferrin receptor. Proc. Natl. Acad. Sci. U. S. A. 110, 8662-8667 (2013).
44. E. Nance, S. H. Pun, R. Saigal, D. L. Sellers, Drug delivery to the central nervous system. Nat. Rev. Mater. 7, 314-331 (2022).
45. K. I. Vong, et al., SOX9-COL9A3-dependent regulation of choroid plexus epithelial polarity governs blood-cerebrospinal fluid barrier integrity. Proc. Natl. Acad. Sci. U. S. A. 118, e2009568118 (2021).
46. K. Felgenhauer, “The filtration concept of the blood-CSF-barrier as basis for the differentiation of CSF proteins” in New Concepts of a Blood-Brain Barrier, J. Greenwood, D. J. Begley, M. B. Segal, Eds. (Springer, Boston, MA, 1995), pp. 209-217.
47. Poon, L. H., Kang, G. A. & Lee, A. J. Role of Tetrabenazine for Huntington's Disease-Associated Chorea. Annals of Pharmacotherapy 44, 1080-1089 (2010).
48. Wang, H., Chen, X., Li, Y., Tang, T.-S. & Bezprozvanny, I. Tetrabenazine is neuroprotective in Huntington's disease mice. Mol Neurodegener 5, 18 (2010).
49. Menalled, L. B. et al. Comprehensive behavioral testing in the R6/2 mouse model of Huntington's disease shows no benefit from CoQ10 or minocycline. PLOS One 5, e9793 (2010).
50. R. R. Ribchester, et al., Progressive abnormalities in skeletal muscle and neuromuscular junctions of transgenic mice expressing the Huntington's disease mutation. Eur. J. Neurosci. 20, 3092-3114 (2004).
51. Liao, Y.-H., Chang, Y.-J., Yoshiike, Y., Chang, Y.-C. & Chen, Y.-R. Negatively Charged Gold Nanoparticles Inhibit Alzheimer's Amyloid-β Fibrillization, Induce Fibril Dissociation, and Mitigate Neurotoxicity. Small 8, 3631-3639 (2012).
52. Xiao, W., Wang, R.-S., Handy, D. E. & Loscalzo, J. NAD(H) and NADP(H) Redox Couples and Cellular Energy Metabolism. Antioxid Redox Signal 28, 251-272 (2017).
53. Geoffrey M Cooper. The Mechanism of Oxidative Phosphorylation. in The Cell (Sinauer Associates, Sunderland, MA, 2000).
54. Wang, Z. et al. A Mechanism Underpinning the Bioenergetic Metabolism-Regulating Function of Gold Nanocatalysts. Small 20, 2304082 (2024).
55. Estevez-Fraga, C. et al. Genetic topography and cortical cell loss in Huntington's disease link development and neurodegeneration. Brain 146, 4532 (2023).
56. Naia, L. et al. Histone Deacetylase Inhibitors Protect Against Pyruvate Dehydrogenase Dysfunction in Huntington's Disease. J Neurosci 37, 2776-2794 (2017).
57. Sorbi, S., Bird, E. D. & Blass, J. P. Decreased pyruvate dehydrogenase complex activity in Huntington and Alzheimer brain. Ann Neurol 13, 72-78 (1983).
58. Ayala, A., Muñoz, M. F. & Argüelles, S. Lipid peroxidation: production, metabolism, and signaling mechanisms of malondialdehyde and 4-hydroxy-2-nonenal. Oxid Med Cell Longev 2014, 360438 (2014).
59. Bullón, P. et al. Lipophilic antioxidants prevent lipopolysaccharide-induced mitochondrial dysfunction through mitochondrial biogenesis improvement. Pharmacol Res 91, 1-8 (2015).
60. Perea, J. R. et al. p38 activation occurs mainly in microglia in the P301S Tauopathy mouse model. Sci Rep 12, 2130 (2022).
61. D'Mello, S. R. When Good Kinases Go Rogue: GSK3, p38 MAPK and CDKs as Therapeutic Targets for Alzheimer's and Huntington's Disease. Int J Mol Sci 22, (2021).
62. Fan, J. et al. P38 MAPK is involved in enhanced NMDA receptor-dependent excitotoxicity in YAC transgenic mouse model of Huntington disease. Neurobiol Dis 45, 999-1009 (2011).
63. Chen, Z. S. et al. Mutant GGGGCC RNA prevents YY1 from binding to Fuzzy promoter which stimulates Wnt/β-catenin pathway in C9ALS/FTD. Nat Commun 14, 8420 (2023).
64. Godin, J. D., Poizat, G., Hickey, M. A., Maschat, F. & Humbert, S. Mutant huntingtin-impaired degradation of beta-catenin causes neurotoxicity in Huntington's disease. EMBO J 29, 2433-2445 (2010).
65. Vallée, A., Lecarpentier, Y., Guillevin, R. & Vallée, J.-N. Aerobic glycolysis in amyotrophic lateral sclerosis and Huntington's disease. Rev Neurosci 29, 547-555 (2018).
66. Chan, C. K. W. et al. A sub-10-nm, folic acid-conjugated gold nanoparticle as self-therapeutic treatment of tubulointerstitial fibrosis. Proc Natl Acad Sci U S A 120, e2305662120 (2023).
67. Yuan, J. & Ofengeim, D. A guide to cell death pathways. Nat Rev Mol Cell Biol 25, 379-395 (2024).
68. Lee, S.-R. & Lo, E. H. Interactions Between p38 Mitogen-Activated Protein Kinase and Caspase-3 in Cerebral Endothelial Cell Death After Hypoxia-Reoxygenation. Stroke 34, 2704-2709 (2003).
69. Paldino, E., D'Angelo, V., Sancesario, G. & Fusco, F. R. Pyroptotic cell death in the R6/2 mouse model of Huntington's disease: new insight on the inflammasome. Cell Death Discov 6, 69 (2020).
70. Paldino, E. et al. Modulation of Inflammasome and Pyroptosis by Olaparib, a PARP-1 Inhibitor, in the R6/2 Mouse Model of Huntington's Disease. Cells 9, (2020).
71. Shen, H. et al. Pyroptosis executive protein GSDMD as a biomarker for diagnosis and identification of Alzheimer's disease. Brain Behav 11, e02063 (2021).
72. Meyers, A. K. et al. Pyruvate dehydrogenase kinase supports macrophage NLRP3 inflammasome activation during acute inflammation. Cell Rep 42, 111941 (2023).
73. Sharma, B. R. & Kanneganti, T.-D. NLRP3 inflammasome in cancer and metabolic diseases. Nat Immunol 22, 550-559 (2021).
74. Yang, K., Li, B. & Chen, J. Knockdown of phosphoinositide-dependent kinase 1 (PDK1) inhibits fibrosis and inflammation in lipopolysaccharide-induced acute lung injury rat model by attenuating NF-κB/p65 pathway activation. Ann Transl Med 9, 1671 (2021).
75. Björkqvist, M. et al. A novel pathogenic pathway of immune activation detectable before clinical onset in Huntington's disease. J Exp Med 205, 1869-1877 (2008).
76. Kho, A. R. et al. The Effects of Sodium Dichloroacetate on Mitochondrial Dysfunction and Neuronal Death Following Hypoglycemia-Induced Injury. Cells 8, (2019).
77. Kasparová, S. et al. Effect of coenzyme Q10 and vitamin E on brain energy metabolism in the animal model of Huntington's disease. Neurochem Int 48, 93-99 (2005).
78. Zhang, X. et al. The Association Between Folate and Alzheimer's Disease: A Systematic Review and Meta-Analysis. Front. Neurosci. 15, 661198 (2021).
79. Laich, A. et al. Moderate hyperhomocysteinemia in patients with Huntington's disease. Pteridines 13, 121-125 (2002).
80. Calderón Guzmán, D. et al. Folic acid increases levels of GHS in brain of rats with oxidative stress induced with 3-nitropropionic acid. Arch. Physiol. Biochem. 126, 1-6 (2018).
81. Alam, C., Kondo, M., O'Connor, D. L. & Bendayan, R. Clinical Implications of Folate Transport in the Central Nervous System. Trends Pharmacol Sci 41, 349-361 (2020).
82. Araújo, J. R., Gonçalves, P. & Martel, F. Characterization of uptake of folates by rat and human blood-brain barrier endothelial cells. Biofactors 36, 201-209 (2010).
83. Balashova, O. A., Visina, O. & Borodinsky, L. N. Folate action in nervous system development and disease. Dev Neurobiol 78, 391-402 (2018).
84. Bobrowski-Khoury, N., Sequeira, J. M. & Quadros, E. V. Brain Uptake of Folate Forms in the Presence of Folate Receptor Alpha Antibodies in Young Rats: Folate and Antibody Distribution. Nutrients 15, (2023).
85. Grapp, M. et al. Choroid plexus transcytosis and exosome shuttling deliver folate into brain parenchyma. Nat Commun 4, 2123 (2013).
86. An, Y. et al. Dietary intakes and biomarker patterns of folate, vitamin B6, and vitamin B12 can be associated with cognitive impairment by hypermethylation of redox-related genes NUDT15 and TXNRD1. Clin Epigenetics 11, 139 (2019).
87. Bogdanov, M. B., Andreassen, O. A., Dedeoglu, A., Ferrante, R. J. & Beal, M. F. Increased oxidative damage to DNA in a transgenic mouse model of Huntington's disease. J Neurochem 79, 1246-1249 (2001).
88. J. Lipka, et al., Biodistribution of PEG-modified gold nanoparticles following intratracheal instillation and intravenous injection. Biomaterials 31, 6574-6581 (2010).
89. M. Hammarlund-Udenaes, M. Fridén, S. Syvänen, A. Gupta, On the rate and extent of drug delivery to the brain. Pharm. Res. 25, 1737-1750 (2008).
90. M. Xu, et al., How entanglement of different physicochemical properties complicates the prediction of in vitro and in vivo interactions of gold nanoparticles. ACS Nano 12, 10104-10113(2018).
91. B. Ouyang, et al., The dose threshold for nanoparticle tumour delivery. Nat. Mater. 19, 1362-1371(2020).
92. K. Hou, et al., Chiral gold nanoparticles enantioselectively rescue memory deficits in a mouse model of Alzheimer's disease. Nat. Commun. 11, 4790 (2020).

We claim:

1. A self-therapeutic composition for treating neurodegenerative diseases, the self-therapeutic composition comprising a drug carrier adapted for crossing the blood-brain barrier (BBB) for delivery in an extracellular brain space of a subject and for inhibiting renal or liver clearance in the subject, wherein the drug carrier comprises a metal nanoparticle (NP), wherein the nanoparticle comprises a metal core and a coating comprising a hydrophilic polymer and optionally a therapeutic agent comprising a linker conjugated to the hydrophilic polymer, wherein the linker comprises functional groups for binding to the metal core and to the polymer.

2. The composition of claim 1, wherein the metal NP comprises a gold core and wherein the hydrophilic polymer coating comprises thiolated polyethylene glycol (PEG) strands.

3. The composition of claim 2, wherein the metal NP is a (PEG)-coated gold NP comprising Au₃@PEG₁ₖ NP or Au₁₃@PEG₁ₖ NP.

4. The composition of claim 3, wherein the (PEG)-coated gold NP has a size of about from 10 to about 25 nm, wherein the gold core has a size of about from 2.7 to about 12.7 nm.

5. The composition of claim 3, wherein the gold core has a size of about 3-nm or about 13-nm, wherein the (PEG)-coated gold NP has a size of about 11-nm to 25-nm.

6. The composition of claim 2, wherein the PEG coating comprises not less than an average of 3.5 thiolated PEG strands per nm² of gold surface.

7. The composition of claim 2, wherein the PEG-coated gold NP strands are conjugated with Folic Acid (FA) to form Au₃@PEG₁ₖ@FA NP.

8. The composition of claim 7, wherein a loading density of HS-PEGI-FA on each Au NP is about 29.0±5 strands.

9. A method for treating neurodegenerative diseases, the method comprising administering an effective amount of the composition of claim 1 to a subject with a neurodegenerative diseases, comprising Huntington's disease, Alzheimer's disease or Parkinson's disease.

10. The method of claim 9, wherein the metal NP is Au₃@PEG₁ₖ NP or Au₁₃@PEG₁ₖ NP.

11. The method of claim 9, wherein the metal NP is Au₃@PEG₁ₖ@FA NP.

12. The method of claim 11, wherein the dosage of Au₃@PEG₁ₖ @FA NP administered to a subject ranges from about 0.1 mg/kg/day to about 0.5 mg/kg/day.

13. The method of claim 12, wherein the composition reduces oxidative stress and neuroinflammation by reducing 8-OHdG level in the striatum, TNF-α, IL-1B, IL-4, and IL-6 in cortex, and IL-4 in striatum of the subject.

14. The method of claim 9, wherein the composition is administered once weekly or monthly for about 1 year to about 10 years.

15. The method of claim 9, wherein the composition is administered intravenously (i.v.), via inhalation, transnasally, orally, sublingually, enterally, and/or parenterally.

16. The method of claim 9, wherein the subject is a mammal.

17. The method of claim 15, wherein the mammal is a human.

18. The method of claim 15, wherein the composition is administered intravenously for delivery into the cortex and striatum in a subject with a neurodegenerative disease, comprising Huntington's disease, Alzheimer's disease or Parkinson's disease.

19. The method of claim 18, wherein the composition comprising Au₃@PEG₁ₖ NP reduces pyroptosis by inhibiting p38α mitogen-activated protein kinase (MAPK) phosphorylation and pyruvate dehydrogenase kinase 1 (PDK1) in the brain of the subject.

20. A method of synthesizing a gold nanoparticle for treating neurodegenerative diseases, the method comprising conjugating a gold NP with polyethylene glycol (PEG) and further conjugating a therapeutic agent with a linker to the PEG, wherein the gold nanoparticle is adapted for delivering the therapeutic agent to a therapeutic target.


SEQUENCE LISTING (4 sequences)

SEQ ID NO: 1 - DNA, 20 bp, synthetic construct
  source 1..20: mol_type = other DNA
ccgctcaggttctgctttta

SEQ ID NO: 2 - DNA, 20 bp, synthetic construct
  source 1..20: mol_type = other DNA
tggaaggacttgagggactc

SEQ ID NO: 3 - DNA, 24 bp, synthetic construct
  source 1..24: mol_type = other DNA
ctaggccacagaattgaaagatct

SEQ ID NO: 4 - DNA, 25 bp, synthetic construct
  source 1..25: mol_type = other DNA
gtaggtggaaattctagcatcatcc